(19) United States
(12) Patent Application Publication
(10) Pub. No.: US 2025/0101485 A1
(43) Pub. Date: Mar. 27, 2025

(54) METHODS OF PRODUCING CAROTENOIDS FROM ACID WHEY

(71) Applicants: Massachusetts Institute of Technology, Cambridge, MA (US); Agricultural Genomics Institute at Shenzhen, Chinese Academy of Agricultural Sciences, Shenzhen (CN)

(72) Inventors: Gregory Stephanopoulos, Winchester, MA (US); Yongshou Ma, Quincy, MA (US); Konstantinos Katsimpouras, Cambridge, MA (US); Junichi Mano, Tsukubamirai, Ibaraki (JP); Adrian Fay, Lincoln, MA (US)

(73) Assignees: Massachusetts Institute of Technology, Cambridge, MA (US); Agricultural Genomics Institute at Shenzhen, Chinese Academy of Agricultural Sciences, Shenzhen (CN)

(21) Appl. No.: 18/833,825

(22) PCT Filed: Jan. 26, 2023
(86) PCT No.: PCT/US2023/061361

§ 371 Date: Jul. 26, 2024

Related U.S. Application Data
(60) Provisional application No. 63/304,412, filed on Jan. 28, 2022.

(51) Int. Cl. C12P 23/00; C12N 9/02; C12N 9/04; C12N 9/10; C12N 9/12; C12N 9/90; C12N 15/52; C12P 5/00

(52) U.S. Cl. CPC C12P 23/00; C12N 9/0006; C12N 9/0077; C12N 9/0083; C12N 9/1085; C12N 9/1205; C12N 9/1229; C12N 9/90; C12N 15/52; C12P 5/007; C12Y 205/01029; C12Y 205/01081; C12Y 207/01032; C12Y 207/01036; C12Y 207/04026; C12Y 503/03002; C12Y 505/01019; C07K 2319/00

(57) ABSTRACT

Disclosed herein are metabolically engineered cells capable of producing carotenoids from acid whey.

### CROSS-REFERENCE TO RELATED APPLICATIONS

This application claims the benefit under 35 U.S.C. § 119(e) of U.S. Provisional Application No. 63/304,412, filed Jan. 28, 2022, entitled “METHODS OF PRODUCING CAROTENOIDS FROM ACID WHEY,” the entire disclosure of which is hereby incorporated by reference in its entirety.

### REFERENCE TO AN ELECTRONIC SEQUENCE LISTING

The contents of the electronic sequence listing (M065670522W000 -SEQ-KVC.xml; Size: 83,140 bytes; and Date of Creation: Jan. 19, 2023) is herein incorporated by reference in its entirety.

### FIELD

Provided herein are methods and compositions related to producing carotenoids from acid whey.

### BACKGROUND

Acid whey (AW) is a liquid waste by-product whose untreated disposal poses a serious environmental problem due to its high organic content. In the United States it is mainly produced in the manufacture of the increasingly popular Greek yogurt, but there are also other big sources around the world such as the manufacture of paneer. It is estimated that more than 3 million tons of AW are generated in the United States of America each year. This places an economic burden on the dairy industry, but at the same time presents a great opportunity as it can be used for the production of valuable products.

### SUMMARY

The present disclosure relates, at least in part, to methods and compositions for producing carotenoids from acid whey. Aspects of the present disclosure relate to modified yeast cells capable of producing carotenoids from acid whey. In some embodiments, the present disclosure relates to modified yeast cells capable of converting acid whey to pyruvate, either through lactic acid metabolism, glucose metabolism, or galactose metabolism through the Leloir pathway. In some embodiments, the present disclosure relates to modified yeast cells capable of converting pyruvate to lycopene through the mevalonate pathway. In some embodiments, the present disclosure relates to modified yeast cells capable of converting lycopene to astaxanthin and/or lutein. In some embodiments, the modified yeast cells described herein are modified to overcome substrate inhibition. Aspects of the present disclosure relate to a genetically modified yeast cell (modified cell) comprising: a heterologous gene, wherein the heterologous gene encodes an enzyme having beta-galactosidase (LacA) activity; one or more heterologous genes encoding one or more enzymes capable of converting lactic acid to pyruvate; one or more heterologous genes encoding one or more enzymes of the Leloir pathway; and one or more heterologous genes encoding one or more enzymes of the mevalonate pathway. In some embodiments, the modified cell is an oleaginous yeast cell. In some embodiments, the oleaginous cell is a Yarrowia lipolytica cell.

In some embodiments, the one or more heterologous genes encoding one or more enzymes capable of converting lactic acid to pyruvate is/are selected from the group consisting of a lactate transporter (JEN1) and lactate dehydrogenase (LDH). In some embodiments, the one or more heterologous genes encoding one or more enzymes of the Leloir pathway is/are selected from the group consisting of GAL10M, GAL1, GAL7, and GAL10E. In some embodiments, the one or more heterologous genes encoding one or more enzymes of the mevalonate pathway is/are selected from the group consisting of GGPPS, CarRP, and CarB. In some embodiments, the GGPPS is GGPPSxd derived from Xanthophyllomyces dendrorhous, GGPPSsa derived from Sulfolobus acidocaldarius, GGPPStc derived from Taxus canadensis, GGPPSpa derived from Pantoea agglomerans, GGPPSyl derived from Yarrowia lipolytica.

In some embodiments, the modified cell further comprises a heterologous gene encoding an enzyme having lycopene beta cyclase activity. In some embodiments, the enzyme having lycopene beta cyclase activity comprises an amino acid sequence at least 90% identical to the amino acid sequence as set forth in SEQ ID NO: 1. In some embodiments, the enzyme having lycopene beta cyclase activity comprises an amino acid sequence set forth in any one of SEQ ID NOs: 2-4. In some embodiments, the modified cell further comprises a heterologous gene encoding tHMGR, ERG12, IDI, and ERG20 of the Mevalonate (MVA) pathway, and/or Choline Kinase (CK) and Isopentenyl Phosphate Kinase (IPK). In some embodiments, the modified cell further comprises: a heterologous gene encoding an enzyme having beta-carotene ketolase (CrtW) activity; and a heterologous gene encoding an enzyme having beta-carotene hydroxylase (CrtZ) activity. In some embodiments, the enzyme having CrtW activity is fused to the enzyme having CrtZ activity. In some embodiments, the CrtW/CrtZ fusion enzyme comprises a localization signal. In some embodiments, the localization signal targets the CrtW/CrtZ fusion enzyme to the endoplasmic reticulum, peroxisome, and/or lipid bodies.

In some embodiments, the modified cell further comprises a heterologous gene encoding an enzyme having lycopene beta cyclase activity and/or a heterologous gene encoding an enzyme having lycopene epsilon cyclase activity. In some embodiments, the enzyme having lycopene beta cyclase activity comprises an amino acid sequence at least 90% identical to the amino acid sequence as set forth in SEQ ID NO: 1. In some embodiments, the modified cell further comprises a heterologous gene encoding an enzyme having carotenoid hydroxylase 1 (LUT1) activity and/or a heterologous gene encoding an enzyme having carotenoid hydroxylase 5 (LUT5) activity.

In some embodiments, the modified yeast cell described herein in capable of overcoming substrate inhibition. The term “substrate inhibition,” as used herein, refers to the most common deviation from Michaelis-Menten kinetics, occurring in approximately 25% of known enzymes. Substrate inhibition occurs when the concentration of an enzymatic substrate exceeds the optimal parameter and reduces the growth rate of a cell.

Another aspect of the present disclosure relates to a genetically modified yeast cell (modified cell) comprising: a first heterologous gene, wherein the first heterologous gene encodes an enzyme having beta-carotene ketolase (CrtW) activity; and a second heterologous gene, wherein the second heterologous gene encodes an enzyme having beta-carotene hydroxylase (CrtZ) activity; wherein the modified cell produces beta-carotene. In some embodiments, the modified cell is an oleaginous yeast cell. In some embodiments, the oleaginous cell is a Yarrowia lipolytica cell. In some embodiments, the enzyme having CrtW activity is fused to the enzyme having CrtZ activity. In some embodiments, the CrtW/CrtZ fusion enzyme comprises a localization signal. In some embodiments, the localization signal targets the CrtW/CrtZ fusion enzyme to the endoplasmic reticulum, peroxisome, and/or lipid bodies.

Another aspect of the present disclosure relates to a method of converting a carbon source to lycopene and/or beta-carotene, comprising: contacting a modified cell described herein with a carbon source; and incubating the modified cell with the carbon source for a sufficient time to convert the carbon source to lycopene and/or beta-carotene. In some embodiments, the carbon source is acid whey. In some embodiments, the carbon source is converted to lycopene. In some embodiments, the carbon source is converted to beta-carotene.

Another aspect of the present disclosure relates to a method of converting a carbon source to astaxanthin, comprising: contacting a modified cell described herein with a carbon source; and incubating the modified cell with the carbon source for a sufficient time to convert the carbon source to astaxanthin. In some embodiments, the carbon source is acid whey.

Another aspect of the present disclosure relates to a method of converting a carbon source to alpha-carotene, comprising: contacting a modified cell described herein with a carbon source; and incubating the modified cell with the carbon source for a sufficient time to convert the carbon source to alpha-carotene. In some embodiments, the carbon source is acid whey.

Another aspect of the present disclosure relates to a method of converting a carbon source to lutein, comprising: contacting a modified cell described herein with a carbon source; and incubating the modified cell with the carbon source for a sufficient time to convert the carbon source to lutein. In some embodiments, the carbon source is acid whey.

Another aspect of the present disclosure relates to an enzyme having lycopene beta cyclase activity comprising an amino acid sequence set forth in SEQ ID NO: 2. Another aspect of the present disclosure relates to an enzyme having lycopene beta cyclase activity comprising an amino acid sequence set forth in SEQ ID NO: 3. Another aspect of the present disclosure relates to an enzyme having lycopene beta cyclase activity comprising an amino acid sequence set forth in SEQ ID NO: 4.

Each of the limitations of the invention can encompass various embodiments of the invention. It is, therefore, anticipated that each of the limitations of the invention involving any one element or combinations of elements can be included in each aspect of the invention.

This invention is not limited in its application to the details of construction and the arrangement of components set forth in the following description or illustrated in the drawings. The invention is capable of other embodiments and of being practiced or of being carried out in various ways. Also, the phraseology and terminology used in this disclosure is for the purpose of description and should not be regarded as limiting. The use of “including,” “comprising,” or “having,” “containing,” “involving,” and variations of thereof in this disclosure, is meant to encompass the items listed thereafter and equivalents thereof as well as additional items. As used in this specification and the appended claims, the singular forms “a,” “an” and “the” include plural referents unless the content clearly dictates otherwise.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which may be better understood by reference to one or more of these drawings in combination with the detailed description of specific embodiments presented in this disclosure. The accompanying drawings are not intended to be drawn to scale. The drawings are illustrative only and are not required for enablement of the disclosure. For purposes of clarity, not every component may be labeled in every drawing. In the drawings:

FIGS. 1A-1C. FIG. 1A, Consumption of sugars and organic acids present in AW by the engineered Y. lipolytica strain (Lac, Lactose; Glu, Glucose; Gal, Galactose). FIG. 1B, Production of cell biomass and lipids during the fermentation. FIG. 1C, fatty acid composition of the yeast cells.

FIG. 2. Metabolic pathways for the synthesis of carotenoids from lycopene.

FIG. 3. Overview of the metabolic engineering effort required for the biosynthesis of lycopenefrom AW using Y. lipolytica, Lactose hydrolysis involved the introduction of a β-galactosidase (LacA), enhancement of lactate conversion to pyruvate involved the overexpression of a lactate transporter (JEN1) and a dehydrogenase (LDH), acceleration of native galactose metabolism was achieved by overexpressing the Leloir pathway genes, and lycopene biosynthesis involved the introduction of three heterologous genes: geranylgeranyl diphosphate synthase (GGPPxd), phytoene synthase (CarRP), and phytoene desaturase (CarB).

FIG. 4. The engineered subcellular astaxanthin biosynthetic pathway in Yarrowia lipolytica. Cytosolic acetyl-CoA was the common precursor for lipid formation and astaxanthin synthesis. The β-carotene synthesized at endoplasmic reticulum (ER) was sequestered into lipid body (LB) aggregated from triacylglyceride (TAG). The stored TAG was hydrolyzed to free fatty acid (FFA), transported to peroxisome accompanied with β-carotene translocation, and converted into acetyl-CoA through β-oxidation. Genes involved in previous engineered β-carotene biosynthetic pathway in Y. lipolytica are GGPPsa, CarRP, and CarB. CrtW/Z (CrtW and CrtZ) are heterologous subcellular engineering enzymes associated with astaxanthin biosynthesis in this study. GGPPsa, geranylgeranyl diphosphate synthase from Sulfolobus acidocaldarius; CarRP, bi-functional phytoene synthase/lycopene β-cyclase from Mucor circinelloides; CarB, phytoene dehydrogenase from M. circinelloides. CrtW, β-carotene ketolase; CrtZ, β-carotene hydroxylase. IPP, isopentenyl diphosphate; DMAPP, dimethylallyl diphosphate; FPP, farnesyl diphosphate; GGPP, geranylgeranyl diphosphate; FBP, fructose 1,6-bisphosphatase; DHAP, dihydroxyacetone phosphate; G3P, glyceraldehyde 3-phosphate; PA, phosphatidic acid; DAG, diacylglycerol. The dotted arrows indicate multiple catalytic steps.

FIGS. 5A-5B. Optimization of astaxanthin production. FIG. 5A, the astaxanthin biosynthetic pathway starting from β-carotene was constructed by introducing β-carotene ketolase (CrtW) and hydroxylase (CrtZ). Pathway expression was accompanied with colony color change from yellow to red. FIG. 5B, maximization of astaxanthin production by testing CrtW and CrtZ from diverse organisms. The optimal combination of PsCrtW from Paracoccus sp. and HpCrtZ from Haematococcus pluvialis yielded the highest astaxanthin production after 72 hours of shake-flask cultivation. The different construct combinations were indicated below each bar (black circle, was included; blank, was not included). The average and standard deviation (s.d.) of three independent experiments were shown.

FIGS. 6A-6C. Astaxanthin biosynthesis by different fusion constructs in Y. lipolytica. FIG. 6A, astaxanthin biosynthetic pathways. Depending on the order of ketolation and hydroxylation conferred by CrtW and CrtZ, respectively, multiple routes from β-carotene to astaxanthin were possible, which resulted in the formation of different intermediates (indicated by numbers). CrtW, β-carotene ketolase; CrtZ, β-carotene hydroxylase. FIG. 6B, HPLC traces of carotenoids from flask cultivation of astaxanthin producing strain expressing fused CrtW−Z or individual enzymes CrtW+Z. Compared to the YL02 strain harboring individual enzymes CrtW+Z, accumulation of canthaxanthin \textcircled{4} and zeaxanthin (\textcircled{6}) in fused CrtW−Z strain was significantly reduced while intermediate 3′-Hydroxyechinenone (\textcircled{5}) accumulated to higher levels. FIG. 6C, astaxanthin accumulation in strains harboring functional fusion enzymes. Compared to the control strain expressing individual enzymes CrtW+Z, astaxanthin levels in strains expressing the fusion enzymes were significantly elevated. CrtW−Z fusion strain showed further enhanced performance over CrtZ−W fusion type. Linker sequence, GGGGSGGPGS (SEQ ID NO: 5). The average and s.d. of three independent experiments are shown.

FIGS. 7A-7D. Subcellular organelle-engineering further promoted astaxanthin biosynthesis. FIG. 7A, schematic of astaxanthin biosynthesis when targeting fused enzyme CrtW−Z dependent pathway to endoplasmic reticulum (ER), lipid body (LB), and peroxisome by fusion with KDEL, oleosin and SKL sequences, respectively. FIG. 7B, chromatographic carotenoid profiles of the organelle-targeting strains. Compared to strain with cytosolic expression, accumulation of intermediates, especially 3′-Hydroxyechinenone (\textcircled{5}), echinenone (\textcircled{2}) and β-carotene (\textcircled{1}), was significantly reduced in strains with two (ER and LB) or triple (ER, LB and peroxisome) organelles-engineered. FIGS. 7C-7D, assembling the astaxanthin pathway to subcellular organelles further accelerated the conversion of β-carotene to astaxanthin. In addition, simultaneously targeting fusion enzyme CrtW−Z to all three organelles, LB, ER and peroxisome, yielded the highest astaxanthin titer after 72 hours of shake-flask cultivation in YPD medium. The different construct combinations were indicated below each bar (+, with organelle targeting; −, without organelle targeting). The average and s.d. of three independent experiments were shown. Statistical differences were analyzed using the Student's t-test, and P<0.05 was considered to be statistically significant. ***P<0.001; **P<0.01.

FIGS. 8A-8C. Strains engineered for astaxanthin production achieved high titers in fed-batch cultures. FIG. 8A, astaxanthin production by strain YL17 cultivated in YPD medium containing different initial glucose concentration (20, 30, 40, and 50 g/L). FIGS. 8B-8C, fed-batch fermentation profiles of the astaxanthin producing strain YL17 in conical flasks (FIG. 8B) and 3-L bioreactor (FIG. 8C). The average and s.d. of three independent experiments were shown.

FIG. 9. HPLC analysis of carotenoids in the fusion enzyme strains harboring CrtZ−W or CrtW−Z. \textcircled{1}, β-carotene; \textcircled{2}, echinenone; \textcircled{4}, canthaxanthin; \textcircled{5}, 3′-Hydroxyechinenone; \textcircled{6}, zeaxanthin; \textcircled{7}, astaxanthin.

FIGS. 10A-10B. FIG. 10A, the effect of additional copy of fusion enzyme CrtW−Z on astaxanthin production in engineered strains. Each plus (+) symbol indicated one copy of the gene integrated into genome. The average and s.d. of three independent experiments were shown. FIG. 10B, HPLC analysis of carotenoids in the engineered strain YL12 harboring additional copy of fusion enzyme CrtW−Z. \textcircled{1}, β-carotene; \textcircled{2}, echinenone; \textcircled{4}, canthaxanthin; \textcircled{5}, 3′-Hydroxyechinenone; \textcircled{6}, zeaxanthin; \textcircled{9}, astaxanthin.

FIG. 11. Microscopic image of the cells producing β-carotene. Amount of β-carotene was evidently accumulated in the lipid bodies of cell.

FIGS. 12A-12C. Prediction of transmembrane helices in β-carotene biosynthetic enzymes GGPPsa (FIG. 12A), CarRP (FIG. 12B), and CarB (FIG. 12C) using the TMHMM server v. 2.0.

FIG. 13. Subcellular localization of the β-carotene biosynthetic enzymes GGPPsa, CarRP, and CarB by fusion with GFP protein, respectively.

FIG. 14. Chromatographic carotenoid profiles of the engineered strain YL17 cultivated in different medium. The unique difference among the medium was the initial concentration of glucose. YPD20, 20 g/L glucose; YPD30, 30 g/L glucose; YPD40, 40 g/L glucose; YPD50, 50 g/L glucose. \textcircled{1}, β-carotene; \textcircled{2}, echinenone; \textcircled{4}, canthaxanthin; \textcircled{5}, 3′-Hydroxyechinenone; \textcircled{6}, zeaxanthin; \textcircled{9}, astaxanthin.

FIG. 15. Correlation between dry cell weight (DCW) and OD₆₀₀ in astaxanthin-producing cells. DCW was calculated based on the measured OD₆₀₀ and applying the conversion factor.

FIGS. 16A-16D. Lycopene cyclase showed the substrate inhibition effect. FIG. 16A, lycopene inhibited its downstream enzyme, lycopene cyclase, through substrate inhibition. Consequently, a higher lycopene formation rate than its subsequent conversion rate into β-carotene could aggravate the imbalance, leading to the build-up of lycopene. FIG. 16B, after 3 days of fermentation in YPD media, β-carotene levels in strains expressing relevant biosynthetic genes from different sources indicated that the CarB/CarRP pair reached higher levels of performance. FIG. 16C, heterologous overexpression of GGPP synthase from X. dendrorhous (GGPPxd) increased β-carotene production but also led to major accumulation of lycopene, its biosynthetic precursor. ND, not detected. FIG. 16D, measurements of relative lycopene cyclase catalytic activity indicated that the activity of wild type CarRP (or CarR) was biphasic with respect to lycopene concentration, indicating substrate inhibition. By contrast, the CarRP (or CarR) variant Y27R was completely free of substrate inhibition. CarR, truncated CarRP without P domain. For FIGS. 16B-16D, the average and standard deviation (s.d.) of three independent experiments were shown.

FIGS. 17A-17E. Abolishment of substrate inhibition through protein engineering. FIG. 17A, using a predicted protein model, several positions within the R domain (lycopene cyclase) of CarRP were identified as suitable locations for mutation in order to reduce substrate inhibition. Single substitutions were indicated by light spheres whereas double substitutions were indicated by dark spheres. FIG. 17B, β-carotene selectivity was tested on a total of 50 generated variants. Compared to wild type (WT), Y27R, V175W, and T31R-F92W (boxed) showed the significantly increased β-carotene selectivity, suggesting a reduction in substrate inhibition. Data represent the mean value of two independent experiments. FIGS. 17C-17D, compared to the control strain YLMA03 harboring wild type CarRP, the variants showed significantly increased production of β-carotene, along with a decrease in lycopene accumulation (FIG. 17C). In particular, the strain YLMA11 expressing CarRP (Y27R) achieved a titer of 2.38 g/L (FIG. 17C), in addition to a high selectivity of 98% (FIG. 17D). FIG. 17E, the abolishment of substrate inhibition allowed higher fluxes to be channeled through the carotenoid synthesis pathway (through MVA and IUP overexpression), improving β-carotene titers while maintaining the high selectivity. Ultimately, 4.22 g/L β-carotene was produced in YLMA15 with ˜98% selectivity. For cultures using strains containing IUP, 30 mM isoprenol (FIG. 31) was added to the media post-glucose depletion. For FIGS. 17C and 17E, the average and s.d. of three independent experiments were shown.

FIGS. 18A-18G. GGPPS-mediated metabolic flow restrictor effectively relieved substrate inhibition. FIG. 18A, a GGPPS-mediated metabolic flow restrictor varied the amount of flux through the carotenoid synthesis pathway, thus regulating lycopene formation rates. FIG. 18B, changes in GGPPS activity could be achieved by expressing enzymes from different organisms in Y. lipolytica, as indicated by the varying in vivo GGPP synthesis rate. In these experiments, a po1f background strain with no modifications other than GGPPS expression was used. FIG. 18C, compared to the strain expressing GGPPxd, other strains housing lower-activity GGPPSs mitigated the substrate-inhibition effect of lycopene cyclase. The slower formation rates of lycopene prevented its accumulation and hence nearly all lycopene was converted into β-carotene. ND, not detected. FIG. 18D, fermentation time courses indicated that a balanced pathway with the attenuated GGPPsa (YLMA25) led to minimal lycopene build-up throughout the experiment, consistent with YLMA11, which contained the Y27R variant of CarRP. On the contrary, rapid lycopene accumulation was observed in the strain with the exceedingly efficient GGPPxd (YLMA03). FIG. 18E, gene expression cassettes containing the balanced pathway (GGPPsa, CarB, and CarRP) was sequentially introduced into the po1f-T strain for β-carotene production. With higher copy numbers, β-carotene titers increased up to 2.13 g/L, and the selectivity of β-carotene was maximized. FIG. 18F, overexpressing MVA and IUP further improved β-carotene synthesis while maintaining the its high selectivity. FIG. 18G, using the exceedingly efficient GGPPxd to deliberately trigger substrate inhibition, along with a mutated CarRP(E78K), a lycopene-producing strain was constructed, which reached a titer of 2.62 g/L. ND, not detected. For FIGS. 18B-18G, The average and s.d. of three independent experiments were shown.

FIGS. 19A-19D. Balancing acetyl-CoA distribution between lipid and isoprenoid synthesis benefited carotenoid accumulation. FIG. 19A, cytosolic acetyl-CoA was shared between two competing pathways, de novo lipid biosynthesis and the MVA pathway. However, lipid bodies within the cell formed a hydrophobic region in which carotenoids could be sequestered, thus promoting their accumulation. Consequently, both pathways were necessary, and an optimal partitioning of flux was crucial in achieving high carotenoid titers and per-cell content. In addition, intracellular TAGs could be used as a carbon source for acetyl-CoA generation, which in turn provided the building blocks for carotenoids. FIG. 19B, lipid content was dependent on the C/N ratio of the media, and a higher C/N ratio promoted lipid production. FIGS. 19C-19D, β-carotene titer (FIG. 19C) and content (FIG. 19D) were also functions of the media C/N ratio. However, unlike lipid content, which increased monotonically with the C/N ratio, there was an optimum for β-carotene production. The highest titer and per-cell content of β-carotene occurred at a C/N ratio of 9:1 in Y₁₀P₁₀D₅₀ media. For FIGS. 19B-19D, the average and s.d. of three independent experiments were shown.

FIGS. 20A-20C. Cellular lipids drove carotenoid biosynthesis through β-oxidation during stationary phase after glucose depletion. FIG. 20A, monitoring glucose concentration, lipid content, and β-carotene content throughout fermentation revealed that β-carotene continued to increase after glucose was exhausted from the media. Concurrently, intracellular lipids rapidly declined post glucose depletion, which suggested that cells were mobilizing TAGs as the alternative carbon source when glucose was no longer available. FIGS. 20B-20C, tracing carbons from cells cultured in [U-¹³C]glucose and natural abundance stearic acid indicated that β-oxidation could be a source for acetyl-CoA destined for carotenoid synthesis. The average and s.d. of three independent experiments were shown.

FIGS. 21A-21F. Bioreactor fermentation of β-carotene and lycopene engineered Strains. FIGS. 21A and 21D, fermentation profiles of the β-carotene-producing strain YLMA15 (FIG. 21A) and lycopene-producing strain YLMA34 (FIG. 21D) in a 3-L bioreactor. FIGS. 21B and 21E, β-carotene cultures displayed a deep red-orange color after 240 hours cultivation (FIG. 21B), while lycopene cultures displayed a deep red color (FIG. 21E). FIGS. 21C and 21F, microscopic images of cells producing β-carotene (FIG. 21C) and lycopene (FIG. 21F). β-carotene and lycopene were shown to accumulate intracellularly and dispersed throughout the cytoplasm in the most cells. For FIG. 21A and FIG. 21D, the average and s.d. of three independent experiments were shown.

FIG. 22. Scheme of the metabolic pathway leading to the production of β-carotene in Y. lipolytica. The engineered β-carotene biosynthetic pathway involved genes from the mevalonate pathway that were directly upregulated (black, the isopentenol utilization pathway (IUP, white, and β-carotene synthesis (dotted. HMG-CoA, hydroxymethylglutaryl-CoA; MVA, mevalonate; MVAP, mevalonate-5-phosphate; IP, isopentenyl monophosphate; IPP, isopentenyl diphosphate; DMAPP, dimethylallyl diphosphate; GPP, geranyl pyrophosphate; FPP, farnesyl pyrophosphate; GGPP, geranylgeranyl pyrophosphate. tHMGR, truncated HMG-CoA reductase; ERG12, mevalonate kinase; IDI, isopentenyl diphosphate isomerase; ERG20, geranyl/farnesyl diphosphate synthase; GGPPS, GGPP synthase. CrtYB or CarRP, bi-functional phytoene synthase/lycopene β-cyclase; CrtI or CarB, phytoene dehydrogenase. CK, choline kinase; IPK, isopentenyl phosphate kinase.

FIG. 23. Disruption of TRP1 in Y. lipolytica po1f strain via Crispr-Cas9. A single adenine deletion (single underline in position-110 caused a frameshift mutation (double underline which abolished TRP1 activity.

FIGS. 24A-24D. Effects of engineering the lycopene β-cyclase step on β-carotene production. FIG. 24A, CarRP was a bi-functional enzyme with the R domain and P domain conferring lycopene cyclase and phytoene synthase activities, respectively. Two methods were used to isolate cyclase activity: truncation of the CarRP gene after the R domain and a loss of function mutation within the P domain (D409G). FIG. 24B, increasing the copy number of lycopene beta-cyclase did not improve β-carotene synthesis. FIG. 24C, relative gene expression level related to increased CarRP copy number. FIG. 24D, expressing lycopene β-cyclases from various other organisms led to improvements in β-carotene titers. However, the gains were minor and lycopene accumulation was still observed. The average and standard deviation (s.d.) of three independent experiments were shown.

FIG. 25. Intermediate levels involved in β-carotene synthetic pathway. The intracellular concentrations of biosynthetic intermediates from FPP to β-carotene were measured, and lycopene was the only aggregating precursor. Data shown were the average and s.d. of three independent experiments.

FIG. 26. Structural predictions of the lycopene beta-cyclase in CarRP. A schematic of CarRP protein showing that the 6 transmembrane helices were within the R domain (β-cyclase).

FIG. 27. Computational model of R domain (lycopene cyclase) of CarRP. The model having TrRosetta confidence score was 0.79, which suggested a high certainty in structural model.

FIG. 28. Clustering of variants to illustrate distance between sequences. The variants were clustered using PhyML to ensure spread of variants being tested.

FIG. 29. Relative gene expression level related to the CarRP variants. Relative expression levels of gene CarRP in engineered strains harboring mutated CarRP as well as wide type CarRP (WT) were quantified by RT-PCR. ACT1 was used as an internal control gene for normalization. Data shown were the average and s.d. of three independent experiments.

FIG. 30. Spatial mapping of the substitutions removing substrate inhibition. The positions of the success variants were mapped onto the computational model of the lycopene cyclase with Y27R, V175W, and T31R-F92W shown in spheres. All substitutions seemed to be located in same spatial area of the enzyme.

FIG. 31. The effect of isoprenol or prenol on cell growth. Isopentenol isomers isoprenol or prenol were fed to a po1f strain in YPD media at varying concentrations. OD₆₀₀ was measured after 24 hours of cultivation. Based on these results, 30 mM isoprenol or 10 mM prenol was found to be the suitable concentration for all subsequent experiments. Data shown were the average and s.d. of three independent experiments.

FIGS. 32A-32B. Disruption of lycopene cyclase activity in CarRP. FIG. 32A, the E78K mutation in the R domain of CarRP as indicated by dotted rectangle lead to a loss of function of cyclase activity. FIG. 32B, the HPLC chromatographs showed that mutated CarRP^{E78K} completely abolished β-carotene formation.

FIG. 33. Engineering MVA pathway and IUP further promoted lycopene biosynthesis. Overexpressing the native genes in MVA pathway improved lycopene synthesis. Furthermore, additional introduction of IUP in the lycopene-producing strains further improved titers. Data shown were the average and s.d. of three independent experiments.

FIG. 34. Compositions of modified YPD and YNB media used in this study.

FIG. 35. Determination of the optimal initial glucose concentration for β-carotene production. Cells were cultured in Y₅P₁₀Dₙ media where n represented initial glucose concentration. After 3 days of fermentation, the amount of glucose consumed, OD₆₀₀, and β-carotene titers were measured. The optimal initial glucose concentration was found to be 50 g/L. Beyond that, the performance of the strain was adversely affected and glucose consumption rates decreased as well, presumably due to osmotic stress. The average and s.d. of three independent experiments were shown. Statistical differences were analyzed using the Student's t-test, and P<0.05 was considered to be statistically significant. *P<0.05.

FIG. 36. Comparison of cell growth in media with varying C/N ratios. In these experiments, the YLMA15 strain was used and biomass was found to decrease with increasing C/N ratio. Data shown were the average and s.d. of three independent experiments.

FIG. 37. Using the optimized Y₁₀P₁₀D₅₀ media, lycopene titers were further enhanced. Data shown were the average and s.d. of three independent experiments.

FIG. 38. The micro-morphology of YLMA15 cells throughout fermentation. Cells collected at different time points throughout cultivation were visualized under a microscope and the observations were consistent with the fermentation profile. In the presence of glucose in media during the initial 3 days, lipid droplets within cells progressively agglomerated into lipid bodies that sequestered the produced β-carotene. However, due to TAG breakdown, the lipid bodies were no longer visible during the later stages of glucose-depletion, which in turn caused the accumulated β-carotene to be more dispersed throughout the cell.

FIG. 39. The HPLC chromatograph of carotenoids obtained from YLMA15 after fed-batch fermentation. The selectivity of β-carotene was calculated based on the relative contents of lycopene and β-carotene.

FIGS. 40A-40B. Correlation between dry cell weight (DCW) and OD₆₀₀ in β-carotene-producing cells (FIG. 40A), and lycopene-producing cells (FIG. 40B). DCW was calculated based on the measured OD₆₀₀ and applying the conversion factor.

### DETAILED DESCRIPTION

The present disclosure relates to a method of converting industry waste (e.g., dairy waste) to valuable food and feed ingredients (e.g. carotenoids) and/or microbial animal feed using engineered yeast cells. These ingredients can be the naturally occurring products that belong to the family of isoprenoids (also known as terpenoids) and are synthesized mainly by plants. Specifically, the present disclosure describes the synthesis of carotenoid compounds, such as lycopene, beta-carotene, and astaxanthin from dairy industry waste, and metabolic and protein engineering strategies for the enhanced synthesis thereof. Other products of the isoprenoid family can be similarly synthesized from acid whey (AW) waste.

Currently, the two most common uses of AW are either as fertilizer directly added to the soil, or mixed with silage to feed livestock. However, in both cases, the amount of AW that can be used is limited, and these are low-value applications. Conversely, treatment of AW in wastewater treatment facilities adds to the cost of production of many food products. Overall, no good solution exists while a large volume of potential fermentable nutrients such as lactose, galactose, and lactic acid remains unutilized (Menchik, et al. 2019). Moreover, this yellowish AW by-product is unappealing to the food industry as its acidic and salty taste, high levels of ash, and low levels of protein limit its food applications (Lievore, et al. 2015).

Dairy industry companies have been trying to develop alternative approaches to handle AW. For instance, Chobani (Norwich, NY) and Commonwealth Dairy (Brattleboro, VT) use a reverse-osmosis filtration system to recover water from AW and reduce transportation costs. Others, such as General Mills (Minneapolis, MN), use anaerobic digestion to convert AW to methane, which can subsequently be used to cover some of the energy needs of the plant, via methane fed electrical generators. However, these are all low-added value processes and the revenue generated by converting AW to methane has been relatively low. General Mills has also developed methods to either neutralize AW to use it in food products as a bulking agent or a nutrient fortifier (U. S. Patent Pub. 2014/0348981), or to produce oligosaccharides that could serve as soluble fiber in cereals or baked goods (U. S. Patent Pub. 2014/0348979). Danone describes a method to generate AW with stable lactose content to enable a more consistent way of isolating lactose (International Patent No. WO 2016/177701 A1). Arla Food Ingredients (Viby, Denmark) and Ultima Foods (Quebec, Canada) utilize protein solutions and ultrafiltration, respectively, in an attempt to minimize AW generation during food production.

Apart from dairy products companies, many research groups have tried to find ways to utilize AW. For instance, a method for producing glucose/galactose syrup and whey protein from AW has been suggested using a combination of ultrafiltration and acid-catalyzed thermal hydrolysis of lactose (Lindsay et al., 2018). However, this approach requires costly ultrafiltration membranes to isolate whey protein, and high temperatures to hydrolyze lactose. Moreover, undesired acid-catalyzed degradation reactions limit product yields, while the value of the final product is low. Medium-chain carboxylic acids (MCCAs), such as n-caproic acid, have been another group of compounds that has been targeted as a product from AW fermentation using microbiomes (Xu et al., 2018). The use of a single bioreactor resulted in low specificity regarding the production of MCCAs; therefore, a more intricate system had to be used, phasing the microbiomes into different operating conditions. This system employing bioreactors in series adds to the process cost and suffers from challenges in scalability. Recently, AW was used as an alternative growth medium for a microalgae aiming to produce the enzyme β-galactosidase (Bentahar et al., 2019). In another work, researchers attempted to convert lactose present in AW into galactooligosaccharides (GOS), using two commercially available β-galactosidases from Aspergillus oryzae and Kluyveromyces lactis achieving low GOS yields.

Furthermore, several reports mention the use of AW in fermented milk beverages to take advantage of the nutrients found in AW and substitute water (Lievore et al., 2015; Skryplonek,] et al, 2019). However, the salty and sour taste of AW compromises the required traits of the fermented beverage in terms of flavor, aroma, and aftertaste. Additionally, the use of AW in fermented milk products has been reported to change the viscosity and thus the texture of the product resulting in lower preference during sensory evaluation (Lievore et al., 2015). Masking the flavor and odor of AW would require the addition of extra ingredients, albeit at increased costs.

Natural products are a rich source of bioactive molecules whose diverse properties have supported numerous applications in the pharmaceutical, food and flavor-fragrance industries (Atanasov et al., 2015; Cragg, 1998; Dhingra et al., 1999; Dzubak et al., 2006; Zhou et al., 2009). Due to their structural complexity and very low content in natural sources, chemical synthesis of these compounds and extraction from plants have presented particular challenges (Chemler and Koffas, 2008; Martin et al., 2003), prompting efforts for their production by engineered microorganisms. Most metabolic engineering efforts for the production of chemical products mainly focus on manipulating functional reconstitution of metabolic pathways in the cytosol. However, this strategy often results in poor yields, or formation of undesirable by-products, because of intricate cellular metabolism involving extensive cross-talk and elaborate regulatory mechanisms (Ajikumar et al., 2010; Martin et al., 2003). In this regard, the natural intracellular compartmentalization of eukaryotic cells can provide inspiration for the type of metabolic engineering that can successfully address these challenges (Hammer and Avalos, 2017).

The present disclosure also relates to the construction of engineered yeast cells by applying metabolic and protein engineering strategies allowing for the production of intracellular carotenoid compounds at high concentrations using either glucose or AW as feedstock. The carotenoid compounds, after a purification step, can be used as antioxidants (food fortification), food colorants, dietary supplements, feed additives, and in cosmetics or personal care products. Another product can be microbial animal feed enriched with carotenoids.

The main advantages of this technology are: (1) no prior treatment of AW is required and the bioprocess can be conducted under non-sterile conditions, (2) complete utilization of AW generating a water stream free from organic compounds, (3) synthesis of high value-added specialty ingredients and co-production of microbial animal feed, favorable to the process economics, (4) capability of using both dilute and concentrated AW, (5) simple and scalable fermentation process, (6) GRAS host microorganism allowing for the safe implementation of the technology into existing creamery facilities, which also means immediate access to feedstock, (7) footprint comparable to the area occupied by AW storage tanks, and (8) significant revenue increase over the production of food products and cutting costs related to waste treatment and transportation of AW to farms, resulting in up to 38% increase in revenues over the current Greek yogurt manufacture, for example.

The present disclosure also relates to methods of compartmentalizing metabolic pathways within subcellular organelles of yeast. Subcellular organelles have been receiving growing attention due to their unique physicochemical environments, and enzymatic, metabolite and cofactor contents that may offer favorable conditions for the functioning of different metabolic pathways (Ayer et al., 2013; Hammer and Avalos, 2017). Assembling pathways within smaller subcellular compartments not only increases local substrate and enzyme concentrations resulting in faster reaction rates, but also prevents diversion of intermediates to competing pathways (Avalos et al., 2013). To date, most explorations of metabolic pathway compartmentalization in yeast were carried out with the model organism Saccharomyces cerevisiae, including harnessing mitochondria for fuels, chemicals and drugs (Avalos et al., 2013; Farhi et al., 2011; Szczebara et al., 2003; Yuan and Ching, 2016), compartmentalizing pathways in peroxisomes (Sheng et al., 2016; Zhou et al., 2016), targeting the ER and Golgi for fuel and drug production (Thodey et al., 2014), and vacuolar compartmentalization (Bayer et al., 2009). Targeting biosynthetic pathways to these subcellular compartments has benefited the production of the desired products in this organism. On the other hand, despite these advantages of organelle engineering, its full potential in the oleaginous yeast Yarrowia lipolytica remains largely unexplored. There is an opportunity to capitalize on the prospect of pathway compartmentalization in this yeast due to its unique ability to accumulate large amounts of intracellular lipids in the form of lipid droplets.

Astaxanthin, a high-valued carotenoid-derivative pigment, has been the subject of growing interest due to its broad applications in the food, animal feed, nutraceuticals, cosmetics, and pharmaceutical industries (Ambati et al., 2014). These applications are due to its strong antioxidant (Hama et al., 2012), anti-inflammatory (Bennedsen et al., 2000), and anti-cancer activity (Chew et al., 1999). Traditional methods of astaxanthin production include chemical synthesis and extraction from natural sources. However, biosafety concerns with chemical routes and the high cost and variability of products made by the extraction route limit its extensive application (Qi et al., 2020). Alternatively, metabolic pathway engineering for astaxanthin biosynthesis has been successfully demonstrated in various host organisms, generally occurring in the cellular cytoplasm (Diao et al., 2020; Gong et al., 2020; Henke et al., 2018; Jiang et al., 2020; Jin et al., 2018; Kildegaard et al., 2017; Lemuth et al., 2011; Li et al., 2020; Lu et al., 2017; Ma et al., 2016; Nogueira et al., 2019; Park et al., 2018; Qi et al., 2020; Scaife et al., 2009; Scaife et al., 2012; Tramontin et al., 2019; Ukibe et al., 2009; Wang et al., 2017; Zhang et al., 2018; Zhou et al., 2019; Zhou et al., 2017; Zhou et al., 2015). Astaxanthin yields in these engineered microbes are rather low for cost-effective commercialization, mainly owing to poor conversion efficiency of the precursor β-carotene to astaxanthin.

Metabolic engineering approaches for the production of high-value chemicals in microorganisms mostly use the cytosol as general reaction vessel. However, sequestration of enzymes and substrates, and metabolic cross-talk frequently prevent efficient synthesis of target compounds in the cytosol. Organelle compartmentalization in eukaryotic cells suggests ways for overcoming these challenges. In some embodiments, the present disclosure relates to expressing the astaxanthin biosynthesis pathway in sub-organelles of the oleaginous yeast Yarrowia lipolytica. In some embodiments, enzymes of the astaxanthin pathway may be fused together to improve substrate activity and reaction efficiency. In some embodiments, the fusion of two enzymes converting β-carotene to astaxanthin, β-carotene ketolase and hydroxylase, performs better than the expression of individual enzymes. In some embodiments, individual or fusion enzymes of the astaxanthin biosynthesis pathway are expressed in compartments of lipid body, endoplasmic reticulum or peroxisome. In some embodiments, targeting the astaxanthin pathway to subcellular organelles not only accelerates the conversion of β-carotene to astaxanthin, but also significantly decreases accumulation of the ketocarotenoid intermediates.

The present disclosure relates, at least in part, to methods and compositions for producing carotenoids from acid whey. Aspects of the present disclosure relate to modified yeast cells (e.g., oleaginous yeast cells) capable of producing carotenoids from acid whey. The term “oleaginous yeast cell,” as used herein, refers to yeast cells rich in membrane structure and subcellular compartments, which provide a hydrophobic environment ideal for metabolic engineering and the production of industrial products. In some embodiments, the oleaginous yeast cells are oleaginous yeast cells that utilize acetate for cell growth and product synthesis. For example, in some embodiments the oleaginous yeast cells are Yarrowia lipolytica cells. Y. lipolytica is a non-pathogenic oleaginous yeast that can use a variety of carbon sources, including organic acids, hydrocarbons and various fats and oils. The term “oleaginous” refers to a microbe that can accumulate more than 20% of its dry cell weight as lipid (see C. Ratledge et al., Microbial routes to lipids. Biochem Soc Trans. 1989 December; 17(6):1139-41). Exemplary oleaginous cells include yeasts such as Yarrowia lipolytica, Candida 107, Rhodotorula glutinis, Rhodosporidium toruloides, Cryptococcus curvatus, Trichosporon pullulan, Lipomyces lipofer, Schwanniomyces occidentalis and other species from among Yarrowia, Lipomyces, Rhodosporidium and Cryptococcus; oleaginous bacteria such as those Rhodococcus, Acinetobacter and Streptomyces; and oleaginous algae and microalgae.

Aspects of the present disclosure relate to a genetically modified yeast cell (modified cell) comprising: a heterologous gene, wherein the heterologous gene encodes an enzyme having beta-galactosidase (LacA) activity; one or more heterologous genes encoding one or more enzymes capable of converting lactic acid to pyruvate; one or more heterologous genes encoding one or more enzymes of the Leloir pathway; and one or more heterologous genes encoding one or more enzymes of the mevalonate pathway. In some embodiments, the modified cell is an oleaginous yeast cell. In some embodiments, the oleaginous cell is a Yarrowia lipolytica cell. In some embodiments, a polynucleotide comprising the gene is delivered to the cell. In some embodiments, the cell comprises a polynucleotide comprising the gene. In some embodiments, the enzyme encoded by the gene is delivered to the cell. In some embodiments, the cell comprises the enzyme encoded by the gene. In some embodiments, the gene is a heterologous gene. In some embodiments, the polynucleotide is a heterologous polynucleotide. In some embodiments, the enzyme is a heterologous enzyme. The term “heterologous,” as may be used herein, is used interchangeably with the term “recombinant” and the term “exogenous.” A heterologous gene, polynucleotide, or enzyme refers to a gene, polynucleotide, or enzyme that has been introduced to or expressed in a host cell. A heterologous gene is a gene that has been introduced to or expressed in a host cell. A heterologous polynucleotide is a polynucleotide that has been introduced to or expressed in a host cell. A heterologous enzyme is an enzyme that has been introduced to or expressed in a host cell. In some embodiments, the heterologous gene, polynucleotide, or enzyme comes from a different organism or species from the host cell. In some embodiments, the heterologous gene, polynucleotide, or enzyme is a synthetic gene, polynucleotide, or enzyme. In some embodiments, the heterologous gene, polynucleotide, or enzyme is an additional copy of a gene, polynucleotide, or enzyme that is endogenously expressed by the host cell. In some embodiments, a heterologous gene may be modified by a mutation. The term “mutation,” as may be used herein, refers to a change, alteration, or modification to a nucleotide in a nucleic acid as compared to its wild-type sequence. For example, without limitation, mutations may include substitutions, insertions, deletions, or any combination of the same. In some embodiments, there at least one mutation. In some embodiments, there are more than one mutation. In some embodiments, where there is more than one mutation, the mutations are distinct (e.g., not of the same type (e.g., substitutions, insertions, deletions)). In some embodiments, where there is more than one mutation, the mutations are the same (e.g., not of the same type (e.g., substitutions, insertions, deletions)). Additionally, in some embodiments, the mutations result in a frameshift.

Mutations, which as described hereinabove, are regions (e.g., sections, portions, nucleobases, nucleosides, nucleotides) of a given nucleic acid (e.g., DNA, RNA) which differ as compared to their wild-type nucleic acid, will most often be reflected in each strand of a nucleic acid. That is to say that, when a mutation is present in a sample it and its complement will be observed in each strand of the nucleic acid when sequenced. This presents a problem however, when considering that a sample may contain single-stranded portions (e.g., gaps, overhangs), or areas which may instigate strand resynthesis (e.g., nicks). This problem presents because if a damaged base is present in such single-stranded region, or other region which is resynthesized, a damaged base may instruct the synthesis of its complementary strand to include a base which was not originally present in the nucleic acid from which the sample was generated (because damaged bases can affect non-canonical base pairings). The same could happen if one strand contains mismatched bases. In such instances, the mismatch will show a paired match in the re-synthesized complement instead of it's native mismatched base. When this happens, a sequencing of both strands will read a mutation in each of the strands, thus show a mutation, however, this mutation may not be a true reflection of the original nucleic acid. Such mutations are termed “false mutations,” herein. False mutations are mutations which result from the resynthesis of complementary strands of nucleic acid, which do not represent the original (e.g., native, wild-type) complementary strand of nucleic acid from which the sample was obtained.

The terms “wild type” and “native,” as may be used interchangeably herein, are terms of art understood by skilled artisans and mean the typical form of an item, organism, strain, gene, or characteristic as it occurs in nature as distinguished from engineered, mutant, or variant forms.

In some embodiments, the one or more heterologous genes encoding one or more enzymes capable of converting lactic acid to pyruvate is/are selected from the group consisting of a lactate transporter (JEN1) and lactate dehydrogenase (LDH). In some embodiments, the one or more heterologous genes encoding one or more enzymes of the Leloir pathway is/are selected from the group consisting of GAL10M, GAL1, GAL7, and GAL10E.

The Leloir pathway is a metabolic pathway that is known in the art. The Leloir pathway is used by cells for the catabolism of D-galactose. The term “catabolism,” as used herein, refers to the metabolic process of breaking down complex molecules (e.g., D-galactose) in living organisms to form simpler ones (e.g., glucose-1-phosphate). As will be known by a person having ordinary skill in the art, the Leloir pathway converts galactose to glucose-1-phosphate, inter alia, through the enzymatic activities of GAL10M, GAL1, GAL7, and GALE. In some embodiments, the Leloir pathway is used in modified cells to produce pyruvate.

In some embodiments, the one or more heterologous genes encoding one or more enzymes of the mevalonate pathway is/are selected from the group consisting of geranylgeranyl diphosphate synthase (GGPPS), phytoene synthase (CarRP), and phytoene desaturase (CarB). In some embodiments, the GGPPS is GGPPSxd derived from Xanthophyllomyces dendrorhous, GGPPSsa derived from Sulfolobus acidocaldarius, GGPPStc derived from Taxus canadensis, GGPPSpa derived from Pantoea agglomerans, GGPPSyl derived from Yarrowia lipolytica.

The mevalonate (MVA) pathway is another metabolic pathway that is known in the art. The MVA pathway, also known as the isoprenoid pathway or HMG-CoA reductase pathway, is an essential metabolic pathway that produces, inter alia, isopentenyl pyrophosphate (IPP) and dimethylallyl pyrophosphate (DMAP) from acetyl CoA. In some embodiments, IPP and DMAP in the MVA pathway are further metabolized to farnesyl diphosphate (FPP). In some embodiments, FPP in the MVA pathway is further metabolized to geranylgeranyl pyrophosphate (GGPP) by a GGPPs (e.g. GGPPSxd, GGPPSsa, GGPPStc, GGPPSpa, GGPPSyl). In some embodiments, GGPP in the MVA pathway is further metabolized to phytoene by a CarRP. In some embodiments, phytoene in the MVA pathway is further metabolized to lycopene by a CarB.

In some embodiments, the modified cell further comprises a heterologous gene encoding an enzyme having lycopene beta cyclase activity. The amino acid sequence of the lycopene beta cyclase enzyme is provided here as SEQ ID NO: 1:

(SEQ ID NO: 1)
MLLTYMEVHLYYTLPVLGVLSWLSRPYYTATDALKFKFLTLVAFTTASA
WDNYIVYHKAWSYCPTCVTAVIGYVPLEEYMFFIIMTLLTVAFTNLVMR
WHLHSFFIRPETPVMQSVLVRLVPITALLITAYKAWHLAVPGKPLFYGS
CILWYACPVLALLWFGAGEYMMRRPLAVLVSIALPTLFLCWVDVVAIGA
GTWDISLATSTGKFVVPHLPVEEFMFFALINTVLVFGTCAI

In some embodiments, the enzyme having lycopene beta cyclase activity comprises an amino acid sequence at least 90% identical to the amino acid sequence as set forth in SEQ ID NO: 1. The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category.

Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%,at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (e.g., 0.1%), hundredths of a percent (e.g., 0.01%), etc.).

In some embodiments, the lycopene beta cyclase enzyme (SEQ ID NO: 1) is modified or mutated to increase or decrease enzymatic activity. In some embodiments, the In some embodiments, the enzyme having lycopene beta cyclase activity comprises an amino acid sequence set forth in any one of SEQ ID NOs: 2-4. In some embodiments, comprises the following amino acid substitutions relative to SEQ ID NO: 1: Y27R; V175W; T31R; F92W; or T31R and F92W.

The amino acid sequence of one such modified lycopene beta cyclase activity is provided here as SEQ ID NO: 2:

(SEQ ID NO: 2)
MLLTYMEVHLYYTLPVLGVLSWLSRPRYTATDALKFKFLTLVAFTTASA
WDNYIVYHKAWSYCPTCVTAVIGYVPLEEYMFFIIMTLLTVAFTNLVMR
WHLHSFFIRPETPVMQSVLVRLVPITALLITAYKAWHLAVPGKPLFYGS
CILWYACPVLALLWFGAGEYMMRRPLAVLVSIALPTLFLCWVDVVAIGA
GTWDISLATSTGKFVVPHLPVEEFMFFALINTVLVFGTCAI

The amino acid sequence of another such modified lycopene beta cyclase activity is provided here as SEQ ID NO: 3:

(SEQ ID NO: 3)
MLLTYMEVHLYYTLPVLGVLSWLSRPYYTATDALKFKFLTLVAFTTASA
WDNYIVYHKAWSYCPTCVTAVIGYVPLEEYMFFIIMTLLTVAFTNLVMR
WHLHSFFIRPETPVMQSVLVRLVPITALLITAYKAWHLAVPGKPLFYGS
CILWYACPVLALLWFGAGEYMMRRPLAWLVSIALPTLFLCWVDVVAIGA
GTWDISLATSTGKFVVPHLPVEEFMFFALINTVLVFGTCAI

The amino acid sequence of yet another such modified lycopene beta cyclase activity is provided here as SEQ ID NO: 4:

(SEQ ID NO: 4)
MLLTYMEVHLYYTLPVLGVLSWLSRPYYTARDALKFKFLTLVAFTTASA
WDNYIVYHKAWSYCPTCVTAVIGYVPLEEYMFFIIMTLLTVAWTNLVMR
WHLHSFFIRPETPVMQSVLVRLVPITALLITAYKAWHLAVPGKPLFYGS
CILWYACPVLALLWFGAGEYMMRRPLAVLVSIALPTLFLCWVDVVAIGA
GTWDISLATSTGKFVVPHLPVEEFMFFALINTVLVFGTCAI

In some embodiments, the modified cell further comprises a heterologous gene encoding tHMGR, ERG12, IDI, and ERG20 of the Mevalonate (MVA) pathway, and/or Choline Kinase (CK) and Isopentenyl Phosphate Kinase (IPK). In some embodiments, the modified cell further comprises: a heterologous gene encoding an enzyme having beta-carotene ketolase (CrtW) activity; and a heterologous gene encoding an enzyme having beta-carotene hydroxylase (CrtZ) activity. In some embodiments, the enzyme having CrtW activity is fused to the enzyme having CrtZ activity. In some embodiments, the CrtW/CrtZ fusion enzyme comprises a localization signal. In some embodiments, the localization signal targets the CrtW/CrtZ fusion enzyme to the endoplasmic reticulum, peroxisome, and/or lipid bodies. The term “fusion enzyme,” as used herein, refers to an enzymatic protein that comprises two or more separate proteins. In some embodiments, a fusion enzyme is created through the joining of two or more genes that originally encode separate proteins. In some embodiments, two or more genes joined together are translated into a single protein or enzyme. The term “localization signal” refers to a peptide fragment expressed on a protein of interest that mediates the transport of said protein to a target location inside or outside of the cell. In some embodiments, the localization signal is a short peptide fragment. In some embodiments, the localization signal targets the protein to the endoplasmic reticulum. In some embodiments, the localization signal targets the protein to the peroxisome. In some embodiments, the localization signal targets the protein to lipid bodies of the cell.

In some embodiments, the modified cell further comprises a heterologous gene encoding an enzyme having lycopene beta cyclase activity and/or a heterologous gene encoding an enzyme having lycopene epsilon cyclase activity. In some embodiments, the modified cell further comprises a heterologous gene encoding an enzyme having carotenoid hydroxylase 1 (LUT1) activity and/or a heterologous gene encoding an enzyme having carotenoid hydroxylase 5 (LUT5) activity.

Another aspect of the present disclosure relates to a genetically modified yeast cell (modified cell) comprising: a first heterologous gene, wherein the first heterologous gene encodes an enzyme having beta-carotene ketolase (CrtW) activity; and a second heterologous gene, wherein the second heterologous gene encodes an enzyme having beta-carotene hydroxylase (CrtZ) activity; wherein the modified cell produces beta-carotene. In some embodiments, the modified cell is an oleaginous yeast cell. In some embodiments, the oleaginous cell is a Yarrowia lipolytica cell. In some embodiments, the enzyme having CrtW activity is fused to the enzyme having CrtZ activity. In some embodiments, the CrtW/CrtZ fusion enzyme comprises a localization signal. In some embodiments, the localization signal targets the CrtW/CrtZ fusion enzyme to the endoplasmic reticulum, peroxisome, and/or lipid bodies.

Another aspect of the present disclosure relates to a method of converting a carbon source to lycopene and/or beta-carotene, comprising: contacting a modified cell described herein with a carbon source; and incubating the modified cell with the carbon source for a sufficient time to convert the carbon source to lycopene and/or beta-carotene. In some embodiments, the carbon source is acid whey. In some embodiments, the carbon source is converted to lycopene. In some embodiments, the carbon source is converted to beta-carotene. The term “carbon source,” as used herein, relates to any natural or artificial sources of carbon, such as carbon dioxide, methane, or acid whey.

Another aspect of the present disclosure relates to a method of converting a carbon source to astaxanthin, comprising: contacting a modified cell described herein with a carbon source; and incubating the modified cell with the carbon source for a sufficient time to convert the carbon source to astaxanthin. In some embodiments, the carbon source is acid whey.

Another aspect of the present disclosure relates to a method of converting a carbon source to alpha-carotene, comprising: contacting a modified cell described herein with a carbon source; and incubating the modified cell with the carbon source for a sufficient time to convert the carbon source to alpha-carotene. In some embodiments, the carbon source is acid whey.

Another aspect of the present disclosure relates to a method of converting a carbon source to lutein, comprising: contacting a modified cell described herein with a carbon source; and incubating the modified cell with the carbon source for a sufficient time to convert the carbon source to lutein. In some embodiments, the carbon source is acid whey.

Another aspect of the present disclosure relates to an enzyme having lycopene beta cyclase activity comprising an amino acid sequence set forth in SEQ ID NO: 2. Another aspect of the present disclosure relates to an enzyme having lycopene beta cyclase activity comprising an amino acid sequence set forth in SEQ ID NO: 3. Another aspect of the present disclosure relates to an enzyme having lycopene beta cyclase activity comprising an amino acid sequence set forth in SEQ ID NO: 4.

The details of one or more embodiments of the methods and products disclosed herein are set forth in the description below. Other features or advantages of the methods and products disclosed herein will be apparent from the following drawings and detailed description of several embodiments, and also from the appended claims.

The terms “approximately” or “about,” as may be used interchangeably herein, and as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction of (i.e., percentage greater than or percentage less than) the stated reference value unless otherwise stated or otherwise evident from the context (for example, when such number would exceed 100% of a possible value).

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

The methods and products disclosed herein is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

In order that the methods and products described in the present application may be more fully understood, the following examples are set forth. The examples described in this application are offered to illustrate the systems and methods provided in this disclosure and are not to be construed in any way as limiting their scope.

### Example 1. Microbial Engineering for the Production of Carotenoids from Acid Whey

This Example relates to engineering of the oleaginous yeast Yarrowia lipolytica for carotenoid overproduction from acid whey and/or animal feed with different compositions of oils and proteins according to need. Previously, it was shown that the expression of a secreted extracellular/β-galactosidase along with the expression of genes for the acceleration of the native galactose metabolism pathway, rendered Y. lipolytica capable of consuming all sugars in AW (Mano et al., 2020). Herein, the optimal gene combination for lycopene synthesis and two independent strategies that almost completely circumvented substrate inhibition during beta-carotene synthesis is described. Substrate inhibition was undesirable in industrial applications of microbes used for product synthesis. Although several methods had explored to address this limitation, such as enzyme immobilization, two-phase partitioning bioreactor systems, batch substrate-feeding strategy, and protein engineering, most of these solutions were limited to systems where the inhibition was posed by the starting substrate, and were difficult to apply in the context of microbial engineering for chemical production. It should be noted that strategies were initially demonstrated in carotenoid producing strains of Y. lipolytica using glucose as carbon source. Subsequently, the results informed the engineering of yeast cells for the efficient production of carotenoid compounds from AW.

Biosynthesis of Beta Carotene and Lycopene from Glucose

First, a structure-guided protein design, coupled with phylogenetic information, was used to generate protein variants with reduced inhibition. For instance, a single mutation of a lycopene cyclase gene was identified that completely abolished substrate inhibition without reducing enzyme activity, which resulted in a remarkable increase of β-carotene production. The synthesis of β-carotene in Y. lipolytica required the heterologous expression of three genes that encoded the enzymes phytoene synthase, phytoene dehydrogenase, and lycopene cyclase. The geranylgeranyl diphosphate synthase (GGPPS) was also considered as it controlled the flux directed towards carotenoid instead of sterol synthesis. Relevant genes were sourced from eukaryotic organisms and were introduced into the Y. lipolytica po1f strain with TRP1 disruption.

A second approach resulted in similar titers and selectivity of β-carotene by reducing the carbon flow through the carotenoid pathway and thus prevented inhibitory metabolite accumulation to inhibitory levels, contrary to the traditional paradigms of pathway engineering. This was achieved by establishing a geranylgeranyl pyrophosphate synthase (GGPPS)-mediated metabolic valve that regulated the substrate lycopene formation rate, which effectively alleviated substrate inhibition. While this approach reduced flux through the pathway of interest, the gains from suppressing substrate levels and thus maintaining high enzymatic activity overcompensated for any losses in productivity suffered from the flux diversion.

It was also noted that the product profile of β-carotene versus lycopene could be shifted by varying the in vivo GGPPS activity. The high activity of GGPP can be taken advantage of to reconstitute a dedicated lycopene-producing strain. Combining this idea with a specific lycopene cyclase variant, high concentrations of lycopene were successfully produced with undetectable amounts of β-carotene. An additional increase in lycopene production was achieved by overexpressing the MVA pathway and introducing IUP using glucose as substrate.

Previous studies established that the lipophilic nature of carotenoids promoted their storage in the lipid bodies of the cells. Larroude et al. found that an engineered lipid overproducer strain was capable of producing more β-carotene with a titer of 6.5 g/L, while accompanied with the production of 42.6 g/L lipids (Larroude et al., 2018). While providing compatible compartments for hydrophobic isoprenoid accumulation, the de novo formation of TAGs also consumes large amount of carbon source, resulting in limited acetyl-CoA flux into the MVA, product-forming pathway. In this study it was demonstrated that Y. lipolytica's native capacity for TAG accumulation was sufficient for carotenoid sequestration. By balancing the flux distribution between carotenoid and lipid synthesis through C/N ratio adjustments, a larger portion of the acetyl-CoA pool for carotenoid production was preserved, achieving higher titers and per-cell content.

Moreover, it was demonstrated that the carotenoid biosynthesis during glucose-depleted stationary phase was supported by cellular lipid degradation. During fermentation experiments using glucose as substrate, it was found that the content of β-carotene continued to rise during the stationary phase, even after glucose in the media had been depleted. Throughout the fermentation process, lipid content increased initially, reaching a maximum at day 3, after which it rapidly decreased when glucose was fully consumed. Yet, despite the reduction in lipid content, β-carotene content continued to rise well past the point of glucose depletion, suggesting that TAGs were utilized to sustain metabolic activity and carotenoid synthesis. When glucose was still present during the initial 3 days, lipid droplets within cells progressively agglomerated into lipid bodies that sequestered the produced β-carotene. However, it was also evident that the lipid bodies were no longer visible during the later stages of fermentation due to TAG breakdown, which in turn caused the accumulated β-carotene to be more dispersed throughout the cell. It was demonstrated that the acetyl-CoA formed through β-oxidation can support MVA pathway and eventually contribute to β-carotene formation. Therefore, this is likely the mechanism used by the cells to convert TAGs into carotenoids during the glucose-depleted stationary phase.

Biosynthesis of Beta Carotene and Lycopene from AW

After the results were acquired for carotenoid synthesis using glucose as substrate, a Y. lipolytica strain was constructed that could fully consume all the organic molecules found in AW and produce high concentrations of lycopene. The engineering effort included introducing the genes responsible for lactose degradation, galactose assimilation, a high activity GGPP, a phytoene dehydrogenase, and a bi-functional phytoene synthase/lycopene cyclase of eukaryotic origin bearing a single mutation that abolishes the cyclase activity. The flux distribution between lycopene and lipid synthesis was balanced through C/N ratio adjustments, which achieved a maximum lycopene concentration of about 3 g/L with 0.230 mg of lycopene per gram of dry cell weight using untreated AW as substrate. The engineered strain was also capable of consuming concentrated AW. After 14 days all the sugars and organic acids in AW were entirely consumed resulting in a maximum lycopene concentration of 13.4 g/L.

### Biosynthesis of Astaxanthin

Another high-valued carotenoid-derivative pigment is astaxanthin. Astaxanthin has been the subject of growing interest due to its broad applications in the food, animal feed, nutraceuticals, cosmetics, and pharmaceutical industries. These applications are due to its strong antioxidant, anti-inflammatory, and anti-cancer activity.

The downstream astaxanthin biosynthetic pathway from β-carotene was constructed by expressing the CrtW gene encoding β-carotene ketolase and the CrtZ gene encoding β-carotene hydroxylase. To further improve the heterologous pathway, β-carotene ketolases and hydroxylases from diverse organisms were sourced. Given that the main natural sources for astaxanthin synthesis are bacteria and alga, additional CrtWs and CrtZs were specifically screened from such organisms and a CrtW/Z pair was identified that maximized microbial astaxanthin production using the β-carotene overproducing strain. Furthermore, it was determined that increasing the physical proximity between CrtW and CrtZ could minimize the substrate-enzyme distance and increased reaction rates. To do that, active fusions between the CrtW and CrtZ enzymes were created, with the goal of enhancing the contact of products/precursors with the corresponding enzymes through the creation of a closer microenvironment. The fused enzymes were more efficient at producing astaxanthin compared to the individual enzymes. Finally, the expression of the fusion enzymes was investigated when targeted to various subcellular compartments. Initially, the fusion enzyme CrtW−Z was targeted to lipid bodies (LB) by linking it with the oleosin sequence, thus providing an alternative biological route for astaxanthin biosynthesis. The LB-targeted strain gave significantly higher titer astaxanthin compared to the control strain expressing the pathway in the cytosol. Targeting the fusion enzyme to other organelles, such as the endoplasmic reticulum and peroxisomes, further improved astaxanthin titers from glucose, with the simultaneous targeting to all three sub-organelles yielding even better results.

### Other Products

The results described herein demonstrated the technology of engineering the oleaginous yeast Y. lipolytica for the biosynthesis of a variety of high added value products. The technology can also be applied for the production of other products from AW, like lutein, alpha carotene and other members of the isoprenoid pathway. Therefore, the present disclosure relates to a general method for the upgrade of the dairy industry waste to a collection of high added value products that have broad use as food ingredients.

Production of Animal Feed from AW

Production of protein is a natural component of the yeast life cycle and can comprise roughly 40% of the dry weight under certain conditions (Yamada et al., 2005). Lipid production, on the other hand, is dependent on certain nutrition cues and is usually growth phase-dependent (Goncalves et al., 2014). Early in the fermentation, most energy and carbon are utilized for growth and cell division, but as essential nutrients begin to run out (notably nitrogen), cells cease to divide and instead begin to store excess carbon in the form of lipids, which are sequestered in large intracellular droplets. Under certain conditions these lipid bodies can represent more than two-thirds of cell dry weight by the end of the fermentation (Qiao et al., 2015). In the case of AW, available nitrogen is primarily in the form of milk protein, and Yarrowia's access to this nitrogen source can be altered to produce a product with a higher or lower percentage of weight that is lipids.

The ratio of lipid to protein was controlled by utilizing engineered strains of Yarrowia. Two strains of Yarrowia were employed; W29 (non-engineered), which produced a mix of lipids and cell mass, as well as an engineered strain designated ACC-DGA. The ACC-DGA strain was designed to produce a greater quantity of lipids via overexpression of native Yarrowia genes which encode enzymes involved in triacylglycerol biosynthesis (Tai et al., 2013; U.S. Patent Application No. US20130143282A1). These strains also differed in their ability to consume the protein present in AW. ACC-DGA was deficient in production of secreted proteases, and thus was unable to degrade milk proteins. Starting with these two strains a product that was either high in fat, high in protein, or a mixture of the two was generated.

### Example 2. Converting Dairy Industry Waste into Food and Feed Ingredients

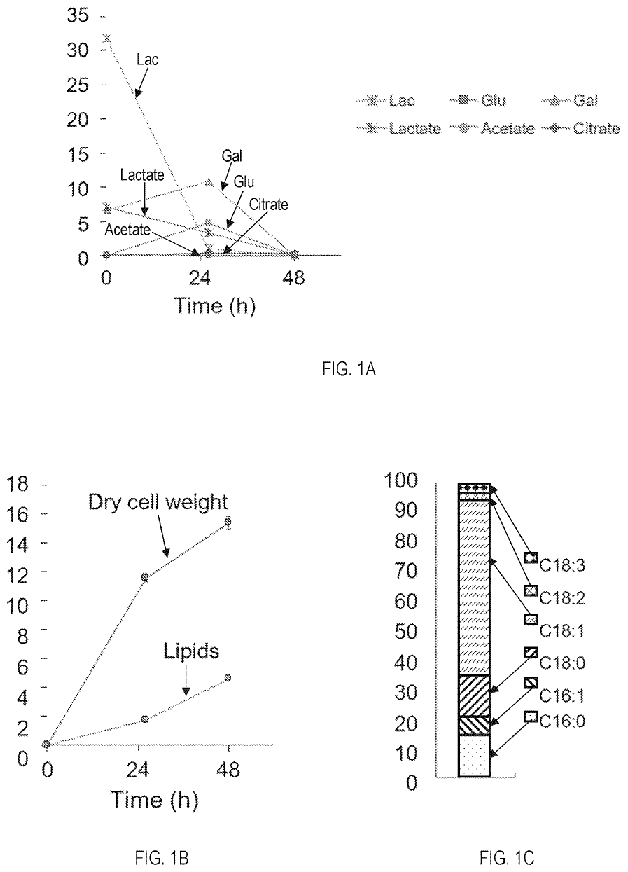

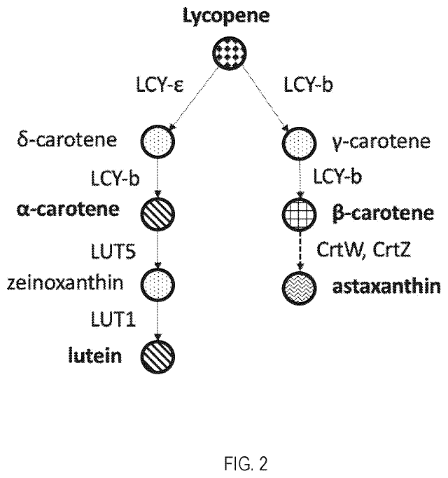

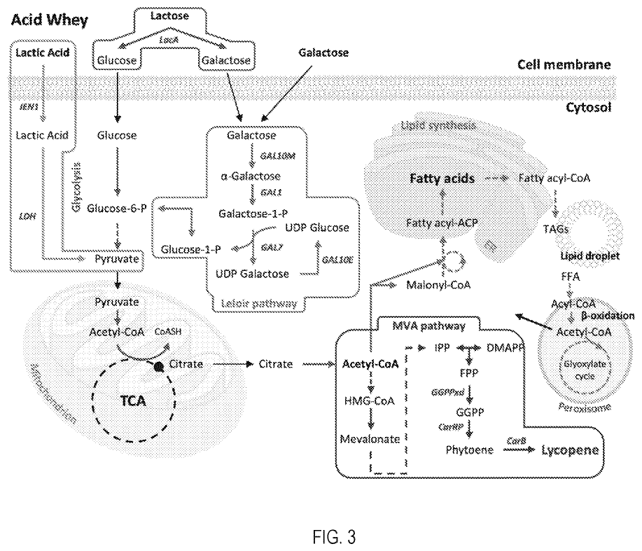

To expand AW utilization technology and transform it into a platform forproducing valuable food and feed ingredients, an engineered strain of Y. lipolytica capable of producing lycopene as a model compound of natural products from AW was sought. It was observed that Lycopene could subsequently serve as a precursor to other carotenoids such as α- and β-carotene, lutein, and astaxanthin (FIG. 2). Previously, attempts to improve the production of f-carotene and astaxanthin, mainly by using the model microorganisms E. coli and S. cerevisiae, had been reported. The recombinant production of α-carotene and lutein in fungi, however, had not been reported. Moreover, based on previous work, it was shown that the expression of a secreted extracellular β-galactosidase along with the expression of genes for the acceleration of the native galactose metabolism pathway, rendered Y. lipolytica capable of consuming all sugars in AW (FIGS. 1A-1C and FIG. 3). Therefore, this AW-utilizing strain was an excellent starting point to use aschassis for subsequent engineeringsteps (FIG. 3).

To achieve this goal, first, the best combination of carotenoidbiosynthetic enzymes that will maximize carotenoid production and accumulation in Yarrowia cells will be determined. For further improvement, different sub-cellularlocalization strategies will be employed to identify the optimal enzyme co-localization in sub-cellular organelles like the endoplasmic reticulum, peroxisome, and lipid bodies. Enzymes localized in different subcellular compartments will have higher activity in converting substrates also localized in the same compartment. Finally, the native mevalonate pathway will be engineered by overexpressing well-known rate-limiting enzymes to increase the supply of carotenoid precursors.

Reverse osmosis has been commonly applied to concentrate AW into smaller volumes for cost reduction in waste treatment and transportation. The high concentration of lactic acid in concentrated AW can inhibit the growth of Y. lipolytica. Thus, methods to enhance tolerance of Y. lipolytica to lactic acid by overexpressing enzymes that are involved in lactic acid consumption will be sought. This approach can be complemented by engineering oxidative stress defense pathways based on previous work where lipid synthesis was improved in Y. lipolytica (Xu et al., 2017). All the constructed strains will be validated in larger volume bioreactors of 10 L. Another approach can be to explore the genome-wide response of Y. lipolytica to concentrated AW that will help to understand the genomic basis of tolerance to concentrated AW. Transcriptional analysis will assess the genome-wide response and allow for the identification of genes central to conferring tolerance along with potential mechanisms underlying enhanced strain tolerance. Using the engineered strains described in the previous aims as starting point, genome-wideevolutionary engineering strategies can be applied and mutants with enhanced tolerance can be isolated. The strain-specific genetic and global gene expression differences of the mutants will be identified using multi-omics analyses (genomics and transcriptomics) and inform the rational engineering of the host strain.

Furthermore, TEA will be employed to evaluate the potential feasibility of the proposed bioprocess and to identify process and economic bottlenecks and targetsfor further research and improvement. Assessment of the overall value of the proposed technology will provide useful information to potential investors. Process modeling will be carried out using a process simulator. Environmental assessment of the proposed bioprocess is another aspect that will be considered to identify and focus on environmentally critical bioprocess parameters (Heinzle et al., 1998).

Additional work also includes the optimization of the fermentation process and purification of the carotenoid products. As intracellular products, carotenoids are sequestered inside the cells and have to be extracted and purified from the cell biomass. The extraction method may initially involve a pretreatment step that helps in the disruption of the cell wall. After that, due to their lipophilic nature, carotenoids are conventionally extracted using organic solvents. The process may include washing steps, a crystallization step, and solvent traces removal by vacuum drying. During carotenoid extraction, a challenge is their sensitivity to excess heat, light, acids, and long extraction times. However, it is noted that separation and purification of carotenoids can be performed following established technologies that can be licensed and deployed in an integrated AW-to-carotenoids scheme.

### Example 3. Targeting Pathway Expression to Subcellular Organelles Improves Astaxanthin Synthesis in Yarrowia lipolytica

Engineering microbes for overproduction of high-value natural products has largely focused on manipulating metabolic pathways in the cellular cytoplasm. Recently there is a growing interest in channeling metabolic pathways in the subcellular organelles of yeast, thus increasing local substrate and enzyme concentrations, and enhancing the efficiency of compartmentalized pathways and production of end-product (Cao et al., 2020; Hammer and Avalos, 2017). Compared to the conventional model yeast S. cerevisiae, systematic investigation of cellular compartments in the oleaginous yeast Y. lipolytica for natural product biosynthesis has lagged. This is so despite the significant role played by intracellular compartments like LB, ER and peroxisome of this yeast. In the present study, the heterologous metabolic pathway for astaxanthin synthesis was assembled using fusion enzymes CrtW−Z and targeting expression in subcellular organelles of Y. lipolytica. The present disclosure relates to bringing in close proximity the precursor of astaxanthin synthesis with the enzymes catalyzing the pathway reactions. As Y. lipolytica is widely regarded as model organism for production of acetyl-CoA-derived compounds (Abdel-Mawgoud et al., 2018), other products derived from the same precursor could benefit from this study.

The first round of experimentation capitalized on the lipophilic nature of the main precursor of astaxanthin synthesis, β-carotene, and targeted the lipophilic compartment of lipid bodies for expression of the astaxanthin pathway. Following successful implementation of this strategy, the compartment of β-carotene synthesis, ER, was targeted next and this increased production further. Finally, the peroxisome was also targeted for compartmentalization owing to serving as storage for lipophilic compounds as well. Targeting the astaxanthin pathway to all three compartments yielded the best results in terms of product accumulation, suggesting that bringing the astaxanthin pathway in close proximity to β-carotene precursor and providing a suitable vehicle for astaxanthin storage were all important for enhanced product accumulation in Y. lipolytica. The simultaneously targeting of CrtW−Z dependent pathway to LB, ER and peroxisome yielded the highest astaxanthin titer reported so far in yeast. Thus, harnessing subcellular organelles can be a promising approach for further enhancing isoprenoid biosynthesis because of their potential advantage of improving precursor supply and cofactors availability.

Overall, the present disclosure has successfully explored enzyme fusion and its compartmentalization in subcellular organelles, effectively channeling substrate or intermediates to end-product. These approaches represent one of the first in developing yeast subcellular cell factory. Hydrophobicity was explored as key property in engineering the astaxanthin pathway, but other molecular features could be explored as well in designing future applications of organelle engineering.

### Screen of β-Carotene Ketolase and Hydroxylase Enzyme Combinations

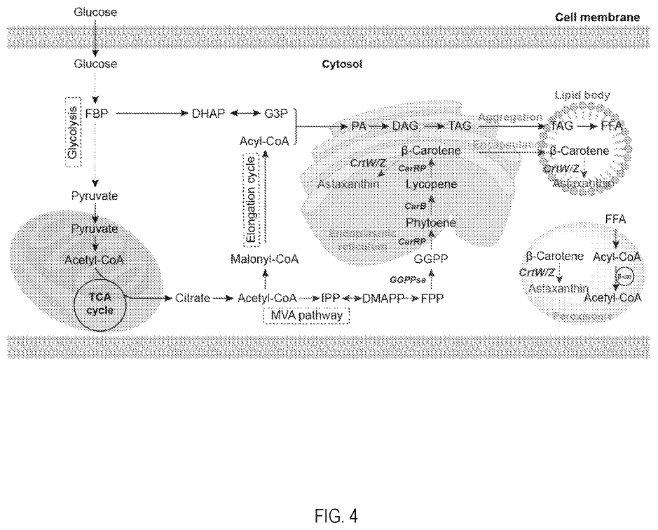

The astaxanthin biosynthetic pathway has been extensively studied and is well characterized. As shown in FIG. 4, glucose is converted into precursors isopentenyl pyrophosphate (IPP) and its isomer dimethylallyl pyrophosphate (DMAPP) through the glycolytic and mevalonate (MVA) pathways. Following that, IPP and DMAPP are condensed to form geranylgeranyl diphosphate (GGPP) by GGPP synthase. GGPP is then converted to astaxanthin by heterologous enzymes comprising the downstream pathway from GGPP to astaxanthin. The heterologous pathway to astaxanthin synthesis can thus be divided into two modules: an upstream module from GGPP to β-carotene and a downstream module from 3-carotene to astaxanthin (FIG. 4). In a previous study, a strain of Y. lipolytica was engineered so that the upstream pathway was capable of overproducing β-carotene. This was done by introducing in Y. lipolytica po1f the genes for the β-carotene biosynthetic pathway: a GGPP synthase from Sulfolobus acidocaldarius (GGPPsa), a gene for the bi-functional enzyme phytoene synthase/lycopene beta-cyclase (CarRP), and a gene for phytoene dehydrogenase (CarB) from Mucor circinelloides. In this β-carotene-producing strain the auxotrophic markers were removed by the Cre-loxP system, allowing for the next round of genome integration. Thus, a chassis host strain was obtained capable of overproducing the substrate β-carotene for astaxanthin synthesis.

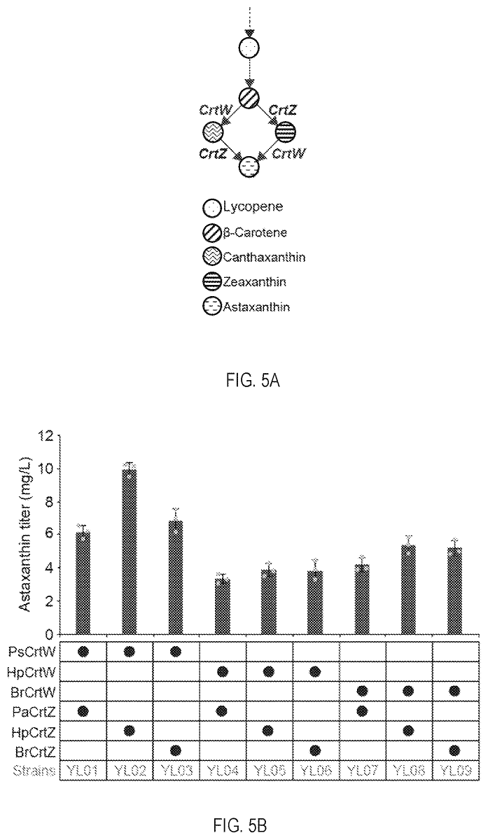

The downstream astaxanthin biosynthetic pathway from β-carotene was constructed by expressing the CrtW gene encoding β-carotene ketolase and the CrtZ gene encoding β-carotene hydroxylase, which perform the addition of two keto moieties and hydroxyl, respectively, changing the colony color from yellow to red (FIG. 5A). The much higher titer of β-carotene, compared to that of astaxanthin, obtained upon heterologous expression of CrtZ/W suggested that β-carotene ketolation and hydroxylation were the rate-limiting steps in astaxanthin synthesis (Kildegaard et al., 2017). In a first attempt to construct the astaxanthin pathway, CrtW from the marine bacterium Paracoccus sp. (PsCrtW) and CrtZ from enterobacteriaceae Pantoea ananatis (PaCrtZ) (Kildegaard et al., 2017) were introduced into the β-carotene producing strain YL00. This yielded 6.1 mg/L of astaxanthin after 72 hours of cultivation (FIG. 5B). The low yield may have been due to an imbalance of enzymatic activities that resulted in intermediate accumulation. It has been recognized that bacterial CrtWs and CrtZs can utilize β-carotene as well as its ketonic and hydroxylated products as substrate, leading to diverse carotenoid intermediate profiles, which can greatly affect astaxanthin yield and productivity (Chang et al., 2015; Choi et al., 2005; Wang et al., 2017). Thus, due to substrate preferences and unbalanced enzymatic activities, an optimal combination of β-carotene ketolase and hydroxylase activities is critical for enhanced astaxanthin accumulation.

To further improve the heterologous pathway, β-carotene ketolases and hydroxylases from diverse organisms were sourced. Given that the main natural sources for astaxanthin synthesis are bacteria and alga, two additional CrtWs and two CrtZs were specifically screened from such organisms (Table 1). To identify a CrtW/Z pair that maximizes microbial astaxanthin production, combinatorically screening of the suite of three CrtWs and three CrtZs in the β-carotene overproducing strain was performed. Astaxanthin titers ranging from 3.2-9.9 mg/L were obtained with the various engineered strains (FIG. 5B), indicating that balancing enzymatic activities was essential for reconstructing an efficient pathway for astaxanthin production. The optimal combination of CrtW/Z yielding the highest astaxanthin production was that from PsCrtW/HpCrtZ (FIG. 5B). To be noted, all strains harboring PsCrtW showed much better performance than strains harboring the other two CrtWs (FIG. 5B). Moreover, HpCrtZ, an enzyme from eukaryotic organisms, was firstly well expressed in Y. lipolytica and yielded high-level of astaxanthin production (FIG. 5B).

### Astaxanthin Production is Enhanced by Fusion Enzymes

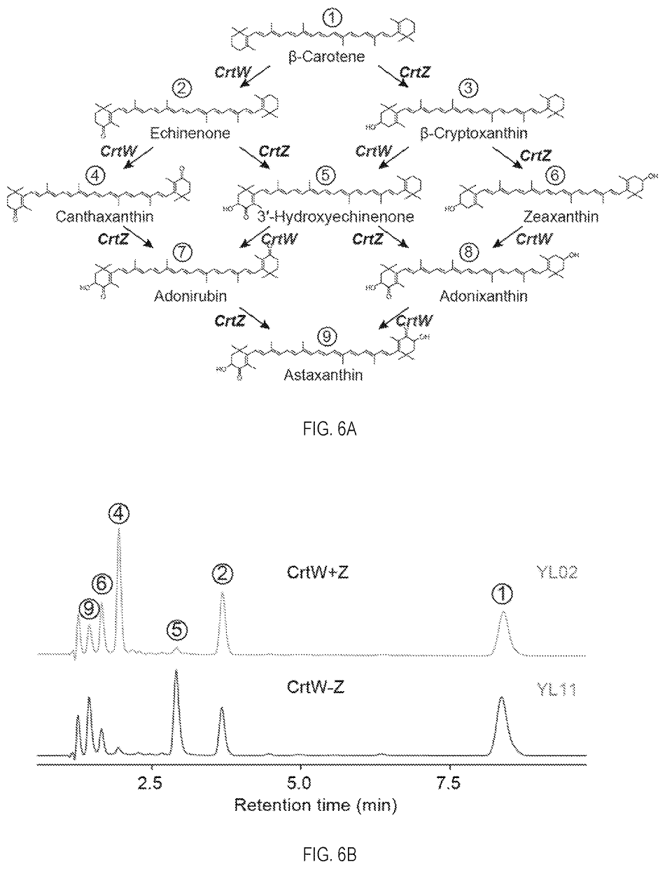

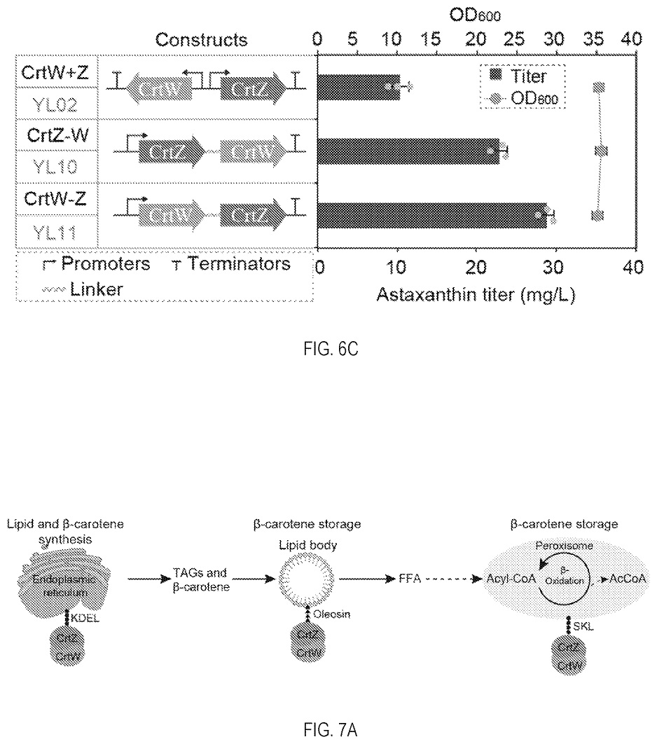

Depending on the order at which the ketolation and hydroxylation reactions were carried out in the biosynthetic process from β-carotene to astaxanthin, several intermediates were synthesized, forming multiple routes from β-carotene to the end product (FIG. 6A). Although the engineered strain YL02 harboring the optimized combination of CrtW and CrtZ improved astaxanthin production, higher levels might be possible by converting other intermediates that also accumulated, especially echinenone, canthaxanthin and zeaxanthin (FIG. 6B). The nature of accumulated intermediates suggested that conversion of canthaxanthin or zeaxanthin to astaxanthin were pathway bottlenecks. It was hypothesized that these bottlenecks were caused by low metabolite concentrations in the vicinity of the CrtZ/CrtW enzymes and could thus be removed by shortening the distance between enzymes and their substrates. Increasing the physical proximity between CrtW and CrtZ could minimize the substrate-enzyme distance and increase reaction rates. To test this hypothesis, active fusions were created between the CrtW and CrtZ enzymes, with the goal of enhancing the contact of products/precursors with the corresponding enzymes through the creation of a closer microenvironment.

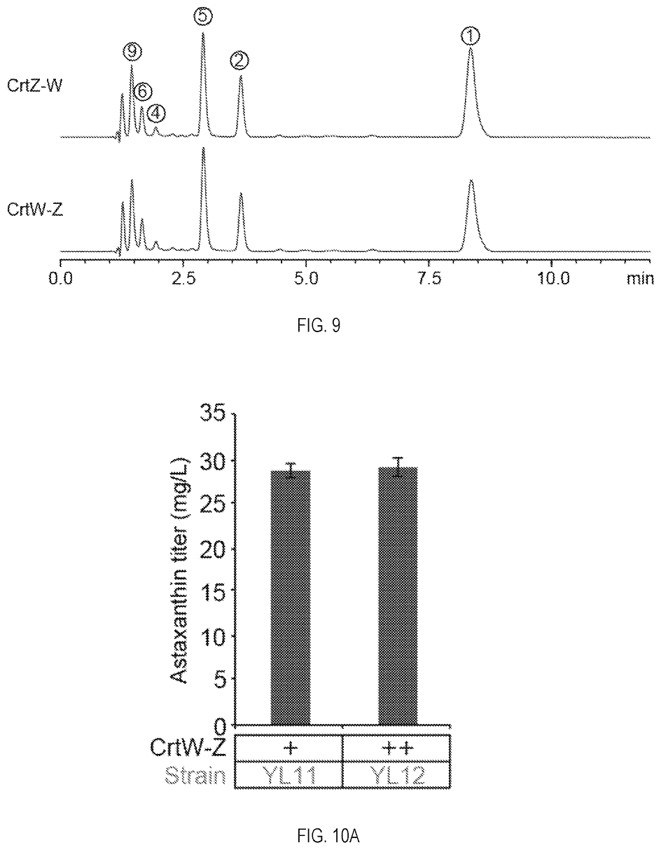

β-carotene ketolase from Paracoccus sp. (PsCrtW) and β-carotene hydroxylase from Haematococcus pluvialis (HpCrtZ) was selected to create fusion enzymes on the basis of their high activity for producing more astaxanthin when expressed individually (FIG. 5B). These two proteins were expressed as translational fusions separated by a linker spacer that was introduced to keep the two enzymes in close proximity and simultaneously allowing interaction between domains (Nogueira et al., 2019). Two constructs were investigated by expressing the fusions as well as their controls in the β-carotene overproducing strain YL00, respectively (FIG. 6C). Levels of astaxanthin and intermediates in the engineered strains were measured and showed that the engineered strains expressing the CrtZ−W or CrtW−Z enzyme fusions could both produce increased amounts of astaxanthin (FIG. 6C and FIG. 9), suggesting that both fused enzymes functioned similarly but were more active than the corresponding individually expressed enzymes. Compared to the control strains harboring individual enzymes CrtW+Z, production of astaxanthin with the fused enzymes strains was significantly improved and increased by 2.2-fold in fusion CrtZ−W strain, up to 22.8 mg/L, and 2.8-fold in fusion CrtW−Z strain, up to 28.9 mg/L (FIG. 6C). This provided support to the hypothesis that the significant improvement of the fusion enzyme strains was likely due to improved local substrate-enzyme proximity and their interactions. Intriguingly, the strain expressing the CrtW−Z fusion showed enhanced performance over the strain with the CrtZ−W fusion enzyme (FIG. 6C), suggesting possibly higher catalytic activity when CrtW was positioned at the N-terminus of the fusion enzyme. It should be noted that the metabolite profiles of the fusion enzyme strains were different from those of the individual enzymes. Intermediate accumulation was reduced with the fusion-enzyme strains, especially canthaxanthin and zeaxanthin (FIG. 6B and FIG. 9). This suggested a faster canthaxanthin and zeaxanthin conversion towards astaxanthin conferred by the fused enzymes. Unexpectedly, the intermediate 3′-Hydroxyechinenone significantly accumulated in the fused enzyme stains (FIG. 6B and FIG. 9). Although the fused enzymes were more efficient at producing astaxanthin compared to the individual enzymes, accumulation of intermediates was not effectively addressed. Hence, a significant challenge was to explore ways to maximize flux from β-carotene to astaxanthin synthesis, without intermediate accumulation.

### Targeting Enzyme Fusions to Subcellular Compartment Enhances Astaxanthin Biosynthesis

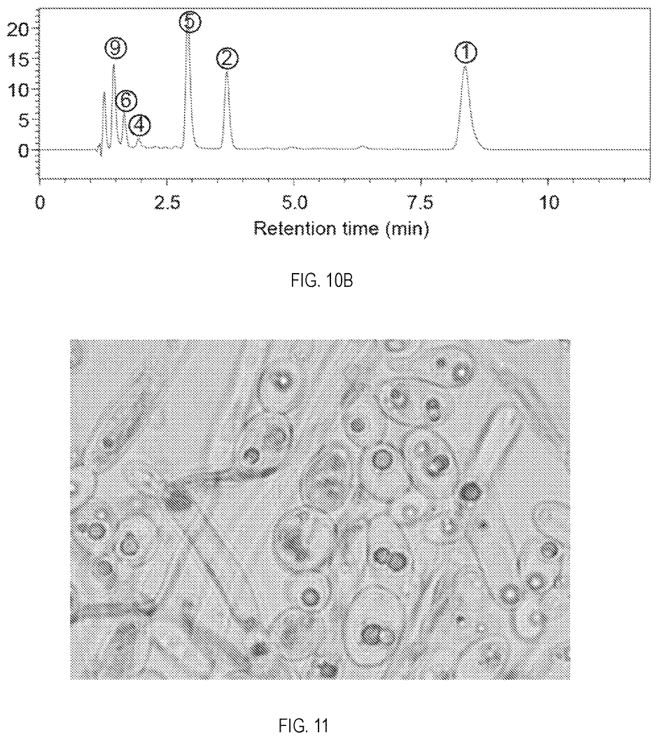

The chromatographic profiles of the carotenoids synthesized by the strain harboring fusion enzyme CrtW−Z (FIG. 6B), showed that the precursor β-carotene, along with the ketocarotenoid intermediates (echinenone, 3′-Hydroxyechinenone and zeaxanthin) accumulated, thus hindering carbon flux towards astaxanthin synthesis. To reduce formation of these intermediates, the effect of increasing the copy number of the astaxanthin biosynthetic pathway was examined by introducing an additional copy of the fusion enzyme CrtW−Z into the astaxanthin producing strain YL11, yielding strain YL12. This, however, did not affect the amount of astaxanthin produced (FIG. 10A), and intermediate accumulation remained largely unaddressed (FIG. 10B). This led to the hypothesis that the inefficient production of astaxanthin is likely due to the sequestration of precursor β-carotene inside lipid droplets, where it cannot be easily accessed by the cytosolic enzymes converting it to astaxanthin.

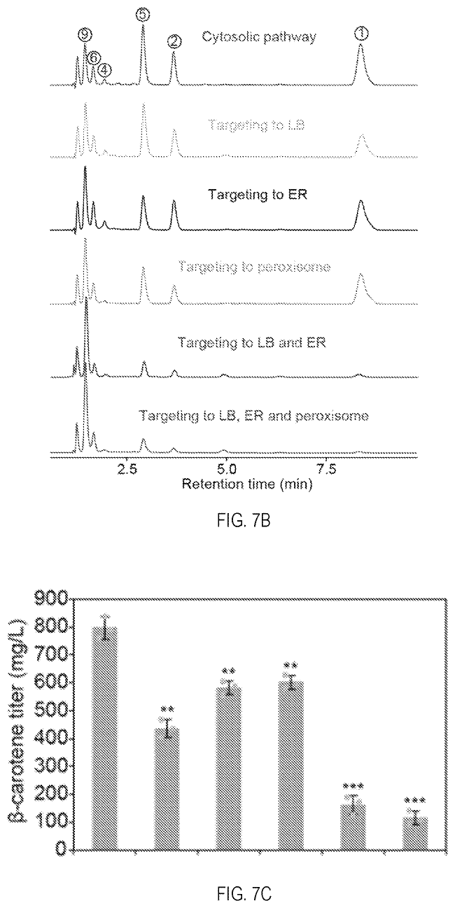

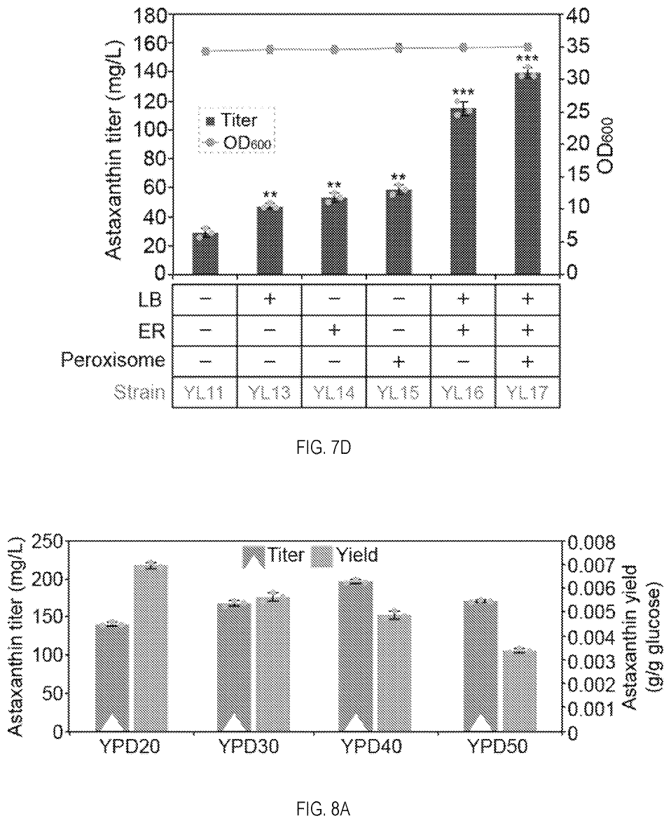

Lipid bodies (LB) in Y. lipolytica indeed create hydrophobic pockets, which can sequester preferentially lipophilic isoprenoid compounds. As the astaxanthin precursor β-carotene is such a lipophilic molecule, whether its sequestration in LB (FIG. 11), could impact astaxanthin production was investigated. It was hypothesized that targeting the astaxanthin pathway to the LB would accelerate conversion of β-carotene to astaxanthin by virtue of greater physical proximity between enzyme and substrate. To this end, well-characterized N-terminal localization signals capable of targeting proteins to LB was utilized (Yang et al., 2019). The fusion enzyme CrtW−Z was targeted to LB by linking it with the protein-location tag oleosin sequence (FIG. 7A), thus providing an alternative biological route for astaxanthin biosynthesis. As expected, the LB-targeted strain YL13 gave a titer of 46.8 mg/L of astaxanthin, a 1.62-fold increase compared to the control strain expressing the pathway in the cytosol (FIG. 7D). Most notably, accumulation of β-carotene, as well as ketocarotenoid intermediates, decreased (FIGS. 7B-7C).

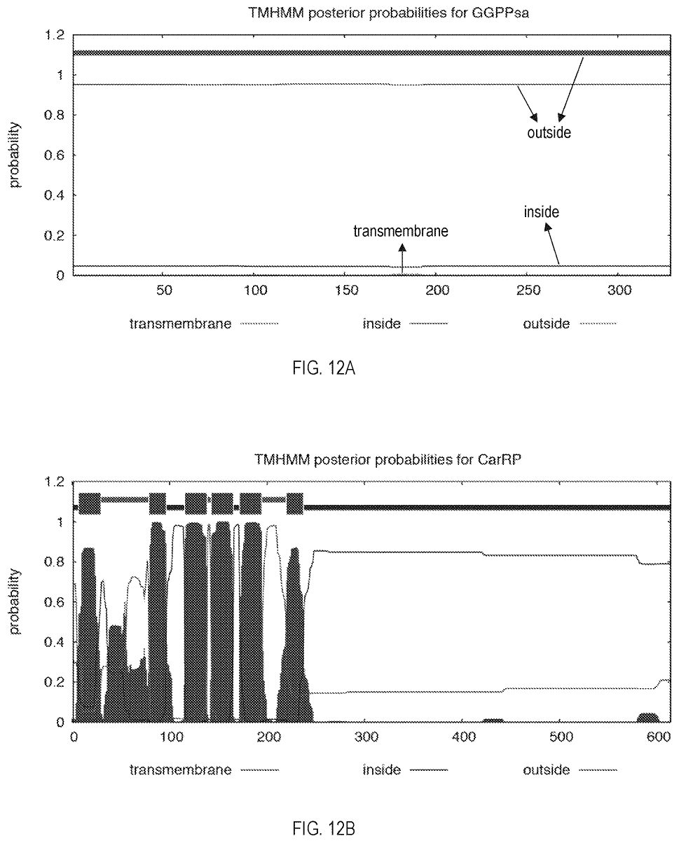

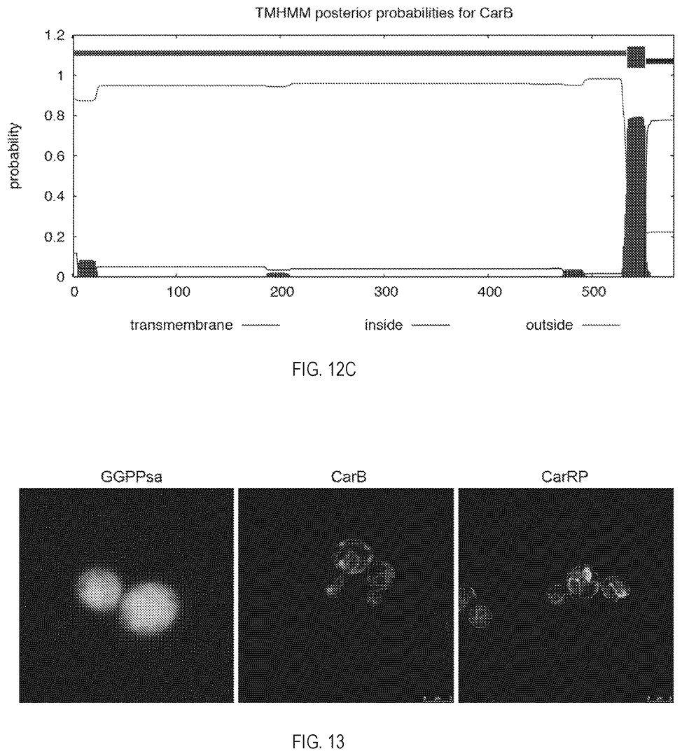

Inspired by these results, the site of β-carotene synthesis in cells was exploited. Prediction of transmembrane helices in the heterologous proteins associated with β-carotene biosynthesis (GGPPsa, CarB and CarRP), revealed that, with exception of GGPPsa, the other two enzymes possess transmembrane helices (FIGS. 12A-12C), suggesting CarB and CarRP were localized at subcellular organelles. To investigate the site of subcellular localization of these three proteins, fluorescent assay was employed from co-localization of the fusion protein with GFP, and found that CarB and CarRP were both localized at the endoplasmic reticulum (ER), while GGPPsa was localized in the cytosol (FIG. 13). This indicated that 3-carotene biosynthesis occurred mostly in the ER, consistent with the synthesis of triacylglycerides (TAGs), which are synthesized in the ER and further agglomerate in the LB (FIG. 7A) (Xu et al., 2016). Therefore, the ER would offer another potential target for astaxanthin pathway compartmentalization.

Besides the LB and ER, another organelle potentially impacting intracellular β-carotene accumulation is the peroxisome that also serves as storage compartment for lipophilic compounds (Liu et al., 2020). Thus, the astaxanthin pathway was targeted, as expressed by the fused enzyme CrtW−Z, to the ER, and peroxisome by fusion with well-characterized targeting sequence KDEL and SKL, respectively (FIG. 7A). As expected, the corresponding organelle-targeted strain significantly increased astaxanthin titer, compared with the strain of cytosolic pathway expression (FIGS. 7B-7D). Strain YL14, harboring the engineered ER-targeted pathway, yielded 53.2 mg/L of astaxanthin after 72 h of cultivation, representing a 1.84-fold increase relative to their cytosolic counterparts (FIG. 7D). The corresponding peroxisome-targeting strain YL15 produced approximately 58.7 mg/L of astaxanthin, an increase of 2.03-fold, compared to that of strain YL11 with cytosolic pathway expression (FIG. 7D).

Considering that a considerable fraction of the β-carotene pool resides in the ER, LB and peroxisome, targeting the pathway to more than one compartment was investigated. It was found that when simultaneously targeting the fusion enzyme CrtW−Z to LB and ER, the astaxanthin titer was significantly increased, compared to that of single subcellular compartmentalization (FIG. 7D). Of note, the bottleneck manifested by β-carotene and ketocarotenoid intermediate accumulation, was effectively relieved (FIGS. 7B-7D). Furthermore, astaxanthin titer was further increased, up to 139.4 mg/L, in the triple organelle-engineering strain YL17 with additional CrtW−Z localized at the peroxisome (FIG. 7D). Taken together, these results support that compartmentalization not only makes intermediates accessible to the downstream engineered biocatalysts, but also increases the catalytic activity of enzymes due to the unique physiochemical environments.

### Optimization of Fermentation Conditions for Maximizing Conversion of β-Carotene to Astaxanthin

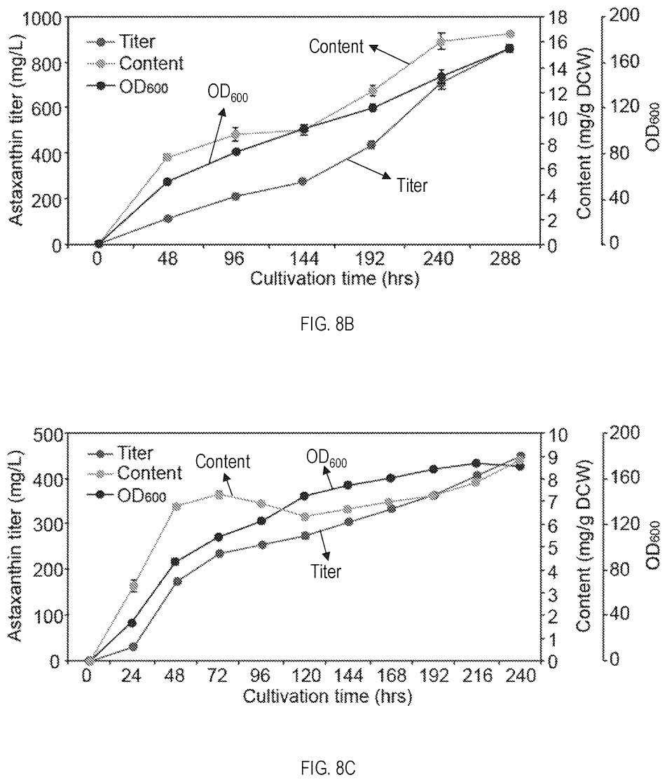

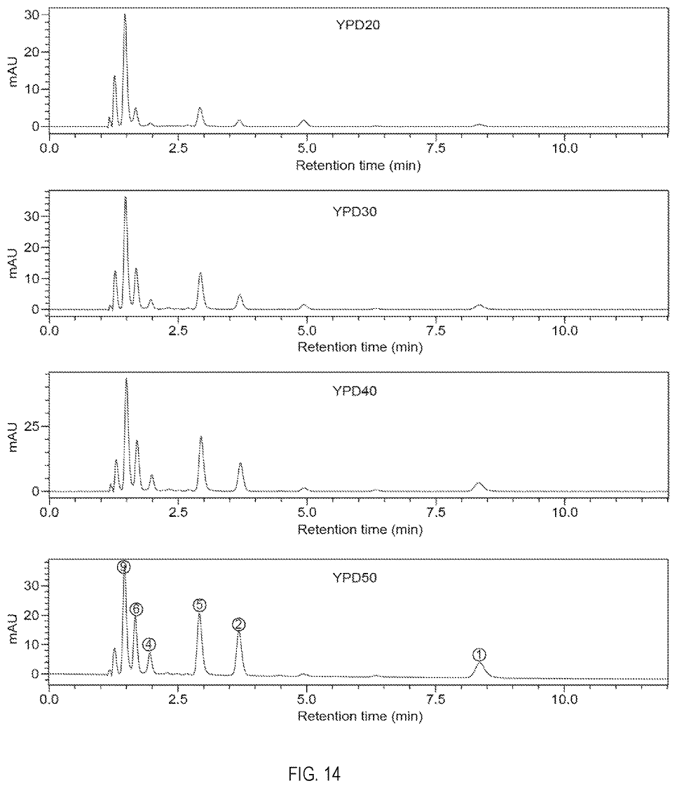

The effect of medium composition on astaxanthin production was investigated next. Glucose concentration was varied while keeping constant the nitrogen amount, thus effectively varying the medium C/N ratio. Large variations in astaxanthin production were observed depending on the initial glucose concentration. It was found that astaxanthin titer gradually increased with increasing glucose concentration, and reached the highest level with YPD40 medium (FIG. 8A). However, astaxanthin yields decreased with increasing initial glucose concentration (FIG. 8A). In addition, a clear positive correlation between initial glucose content and the accumulation of intermediates, including β-carotene and ketocarotenoid intermediates, was observed (FIG. 14). This can be possibly attributed to the increased lipid content that resulted from the higher C/N ratio, and this, in turn, yielded more β-carotene sequestered in lipid body (Larroude et al., 2018), where LB-targeted enzyme turned out to be limited, thus resulting in intermediate accumulation. Based on these results, the YPD20 medium was selected for the demonstration of astaxanthin production in a fed-batch fermentation. Strain YL17 achieved a total astaxanthin titer and content of 858 mg/L and 16.7 mg/g DCW, respectively, in flask fed-batch fermentation (FIG. 8B), which are the highest reported so far in yeast. In addition, the performance of the constructed strains was evaluated by scaling up fermentation to 3-L bioreactors. But only a titer of 453 mg/L of astaxanthin was obtained (FIG. 8C), suggesting further optimization of fermentation in fermenter needed in future.

Anchoring enzymes simultaneously to all three organelles yielded the largest increase of astaxanthin synthesis, and ultimately produced 858 mg/L of astaxanthin in fed-batch fermentation (a 141-fold improvement over the initial strain). The methods and products disclosed herein are expected to help unlock the full potential of subcellular compartments and advance LB-based compartmentalized isoprenoid biosynthesis in Y. lipolytica.

As described above, the present disclosure relates to the fusion expression of two key enzymes in the astaxanthin pathway and the performance of the fusion when targeted to various subcellular compartments. The activity of the key enzymes β-carotene ketolase (CrtW) and hydroxylase (CrtZ) from different sources were assessed and it was found that the PsCrtW/HpCrtZ (sourced from Paracoccus sp. and Haematococcus pluvialis, respectively) pair was best for astaxanthin accumulation. The activities of PsCrtW and HpCrtZ were combined through the creation of enzyme fusion in order to overcome leakage of non-endogenous intermediates. Finally, the above astaxanthin biosynthetic pathway of fused enzymes was targeted to the subcellular compartment of lipid body (LB), alone and in combination with compartmentalization in the endoplasmic reticulum (ER) and peroxisome. Relatively to the cytosolic pathway, channeling the astaxanthin pathway to the organelles yielded significant increase in production as well as decrease of intermediate accumulation. Furthermore, simultaneously targeting the astaxanthin pathway to all three LB, ER and peroxisome yielded the highest production of astaxanthin, and ultimately achieved 858 mg/L (16.7 mg/g DCW) in fed-batch fermentation. These results demonstrate the potential of Y. lipolytica for lipophilic metabolite production by targeting pathway expression to subcellular compartments that allow efficient functioning of the biosynthetic pathways.

These strategies described herein were deployed to maximize production of the carotenoid astaxanthin in Y. lipolytica. First, functional fusions of β-carotene ketolase and hydroxylase (CrtW−Z or CrtZ−W) were created and shown to elevate production of astaxanthin over the level achieved by the individually expressed enzymes (CrtW+Z) in Y. lipolytica. The greatest difference between individually expressed and fusion enzymes, in terms of intermediate accumulation, was the profiles of canthaxanthin and zeaxanthin in CrtW+Z strain while 3′-Hydroxyechinenone in CrtW−Z strain. Canthaxanthin or zeaxanthin are synthesized from β-carotene in two enzymatic steps requiring only CrtW or CrtZ, respectively, while the production of 3′-Hydroxyechinenone requires the participation of both enzymes. This quantitative change of intermediate composition between CrtW+Z and CrtW−Z strains could indicate that canthaxanthin and zeaxanthin are more easily converted into downstream metabolites when both enzymes are fused. The reduction of the intermediate leakage and the acceleration of the overall reaction rates highlight the enhancement of the enzymes interaction when fused together.

### Example 4. Materials and Methods Related to Example 3

### Culture Conditions and Medium

Escherichia coli DH5α cells were grown in Luria-Bertani (LB) medium (BD bioscience) at 37° C. with constant shaking. Corresponding antibiotics (100 g/mL ampicillin and 50 g/mL kanamycin) were added for plasmid selection. All Y. lipolytica strains were cultivated at 30° C. with shaking at 230 rpm. For Y. lipolytica, YPD medium consisted of 10 g/L yeast extract (BD bioscience), 20 g/L peptone (BD bioscience), and 20 g/L glucose (Sigma-Aldrich) was used. Additionally, YNB medium composed of 1.7 g/L yeast nitrogen base (YNB, VWR Life Science), 20 g/L glucose, 5 g/L ammonium sulfate, 15 g/L agar (BD bioscience), and 0.77 g/L appropriate complete supplement mixture without uracil, leucine, or tryptophan (Sunrise science products) was used for selecting transformed Y. lipolytica strains.

### Construction of Plasmids and Strains

E. coli DH5a was used for cloning and plasmid propagation. The Y. lipolytica po1f strain served as the base strain, and all derivatives and plasmids constructed are listed in Table 2. The primers used for plasmid construction are shown in Table 3. All restriction enzymes were purchased from New England Biolabs (NEB). PCR amplification was performed using Q5 high-fidelity DNA polymerase (NEB) or GoTaq DNA polymerase (Promega). PCR fragments were purified using the ZYMO Fragment Recovery Kit (ZYMO research). Plasmids were then constructed from the purified PCR fragments with Gibson Assembly kit (NEB), transformed into chemically competent E. coli cells by heat shock, and extracted using the QIAprep Spin Miniprep Kit (Qiagen). All procedures were performed according to the manufacturer instructions. All engineered Y. lipolytica strains were constructed by transforming linearized plasmids (Not1 digestion) using the lithium-acetate method. Recombinants were verified by PCR amplification from genomic DNA. The astaxanthin biosynthetic genes evaluated in this study were all codon-optimized towards Y. lipolytica.

### Auxotrophic Marker Curation by the Cre-loxP System

Plasmid pYLMA-Cre was transformed into target Y. lipolytica strains to rescue URA3, LEU2 and TRP1 markers. Transformants were selected on YPD agar plate supplemented with a final concentration of 250 mg/L hygromycin B (Sigma-Aldrich). After 2-3 days of cultivation, colonies were transferred onto a new YPD plate containing hygromycin B for 1 more day to allow for more successful marker deletions. Marker curation was confirmed by subculturing the colonies onto YNB-Ura, YNB-Leu, and YNB-Trp agar plates, respectively. Successful removal of all three markers lead to a phenotype conferring uracil, leucine and tryptophan deficiency. Plasmid pYLMA-Cre in cells was then removed by incubating positive strains on YPD agar plates at 30° C. for 24 hours, with 2-3 repeats.

### Shake Flask Fermentations

Single colonies of recombinant strains were picked from plate, inoculated into 2 mL YPD medium, and cultivated overnight (16-18 hours) at 30° C. and 230 rpm. The culture was then transferred to a 50 mL shake flask containing 10 mL YPD medium (initial OD₆₀₀=0.1) and cultivated at 30° C. and 230 rpm for 3 days.

### Quantification of Residual Glucose in Media

500 L sample was extracted from the culture for residual glucose quantification. The cells were centrifuged at 12,000 rpm for 5 minutes, and then the supernatant was filtered through 0.2 μm syringe filters prior to injection into an Agilent technologies 1260 High-Performance Liquid Chromatography (HPLC) equipped with a refractive index detector. A Bio-Rad HPX-87H column was used for separation with 14 mM sulfuric acid as the mobile phase flowing at a rate of 0.7 mL/minute. The injection volume was 10 μL. The column temperature was 50° C.

### Extraction of Carotenoids

Carotenoid extraction was performed as described (Asker, 2017) with the following modification. Briefly, 50˜100 μL culture was centrifuged for 1 minute at 12,000 g, and cell pellets were suspended in 900 μL dimethyl sulfoxide (DMSO) prior to heating at 50° C. for 1 hour until the cells bleached in a water bath. The DMSO extracts were briefly mixed with 450 μL of methanol and centrifuged at 14,000 g for 5 minutes. The resultant supernatants were transferred into glass vials for carotenoid analysis and quantification.

### Analysis and Quantification of Carotenoids

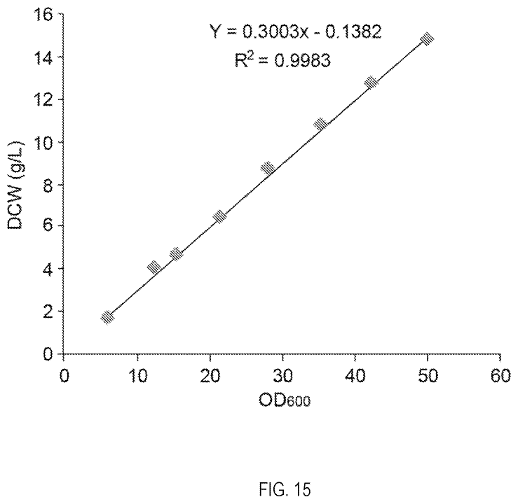

The production of carotenoids was expressed as grams per liter of fermentation broth (g/L) and milligrams per gram of dry cell weight (mg/g DCW). Optical densities were measured at 600 nm with Thermo Spectronic Genesys 20 (Thermo Scientific) and used to calculate cell mass (DCW=0.30×OD₆₀₀, FIG. 15). The analysis and quantification of astaxanthin was performed by HPLC (SHIMADZU LC-20 AT) equipped with a Kromasil C18 column (4.6 mm×250 mm) and UV/VIS detection at 475 nm. The mobile phase consisted of acetonitrile-methanol-isopropanol (5:3:2 v/v) with a flow rate of 1 mL/minute at 40° C. Standard curves of astaxanthin (Sigma-Aldrich) were prepared by running the same extraction process as the samples.

### Targeting Biosynthetic Pathways to Subcellular Compartments

The fusion enzyme CrtW−Z dependent astaxanthin biosynthetic pathways were targeted to different subcellular compartments (ER, LB and peroxisome) using specific addressing signals. The enzymes involved in the astaxanthin biosynthetic pathway were directed to the ER by the addition of a C-terminal KDEL utilizing the following nucleotide sequence 5′-AAGGACGAGCTG-3′ (SEQ ID NO: 6) while removing the stop codon to the end of targeting signal. Similarity, peroxisome or LB targeting of proteins was ensured by the addition of SKL (nucleotide sequence 5′-TCCAAGCTG-3′) or oleosin from Zea mays (codon-optimized oleosin sequence are listed in Table 4), as previously performed in other works on Y. lipolytica engineering (Yang et al., 2019). The fusion enzyme CrtW−Z without any target signal were directed to cytoplasm.

### Fed-Batch Fermentation

Bioreactor fed-batch fermentation was performed in a 3 L fermenter (New Brunswick Bioflo115 system). The initial fermentation was carried out in 1 L of medium containing 100 g/L glucose, 100 g/L peptone, and 50 g/L yeast extract. Temperature was maintained at 30° C. Dissolved oxygen was controlled at 20% of saturation with an agitation cascade of 250˜800 rpm. Air was sparged into the fermenter at 2 vvm. The pH was maintained at 6.8 by feeding 5 M NamH or 5 M HCL. Foam was prevented by the addition of antifoam 204 (Sigma-Aldrich). Fed-batch operation was initiated after 72 hours of cultivation with the 15×YPD medium. Samples were taken every 24 hours to measure OD₆₀₀, glucose and astaxanthin concentrations. Flask fed-batch fermentation was carried out in 50 mL conical flasks with a working volume of 10 mL YPD medium. 15×YPD medium was fed every 48 hours, and pH was not controlled.

TABLE 1
List of ß-carotene ketolase and hydroxylase used in Example 3.

| | Name | | |
|---|---|---|---|
| | used in | | |
| Enzyme | this study | Accession No. | Organism |
| β-carotene | BrCrtW | WP_152949584.1 | Brevundimonas |
| ketolase | HpCrtW | Q39982.1 | Haematococcus pluvialis |
| | PsCrtW | D58420.2 | Paracoccus sp. |
| β-carotene | BrCrtZ | WP_054764844.1 | Brevundimonas |
| hydroxylase | HpCrtZ | KP866868.1 | Haematococcus pluvialis |
| | PaCrtZ | CRH37458.1 | Pantoea ananatis |

TABLE 2
Stains used and constructs in Example 3.

| Stain name | Description of strains and plasmids | Source |
|---|---|---|
| YLMA31 | β-carotene producing strain | Lab stock |
| YL00 | YLMA31, ura⁻/leu⁻/trp⁻ | Example 3 |
| YL01 | YL00, URA3::T_{Lip1}-PsCrtW-P_{EXP1} + P_{TEFin}-PaCrtZ-T_{XPR2} | Example 3 |
| YL02 | YL00, URA3::T_{Lip1}-PsCrtW-P_{EXP1} + P_{TEFin}-HpCrtZ-T_{XPR2} | Example 3 |
| YL03 | YL00, URA3::T_{Lip1}-PsCrtW-P_{EXP1} + P_{TEFin}-BrCrtZ-T_{XPR2} | Example 3 |
| YL04 | YL00, URA3::T_{Lip1}-HpCrtW-P_{EXP1} + P_{TEFin}-PaCrtZ-T_{XPR2} | Example 3 |
| YL05 | YL00, URA3::T_{Lip1}-HpCrtW-P_{EXP1} + P_{TEFin}-HpCrtZ-T_{XPR2} | Example 3 |
| YL06 | YL00, URA3::T_{Lip1}-HpCrtW-P_{EXP1} + P_{TEFin}-BrCrtZ-T_{XPR2} | Example 3 |
| YL07 | YL00, URA3::T_{Lip1}-BrCrtW-P_{EXP1} + P_{TEFin}-PaCrtZ-T_{XPR2} | Example 3 |
| YL08 | YL00, URA3::T_{Lip1}-BrCrtW-P_{EXP1} + P_{TEFin}-HpCrtZ-T_{XPR2} | Example 3 |
| YL09 | YL00, URA3::T_{Lip1}-BrCrtW-P_{EXP1} + P_{TEFin}-BrCrtZ-T_{XPR2} | Example 3 |
| YL10 | YL00, URA3::P_{TEFin}-HpCrtZ + PsCrtW-T_{XPR2} | Example 3 |
| YL11 | YL00, URA3::P_{TEFin}-PsCrtW + HpCrtZ-T_{XPR2} | Example 3 |
| YL12 | YL11, LEU2::P_{TEFin}-PsCrtW + HpCrtZ-T_{XPR2} | Example 3 |
| YL13 | YL00, URA3::P_{TEFin}-PsCrtW + HpCrtZ + oleosin-T_{XPR2} | Example 3 |
| YL14 | YL00, URA3::P_{TEFin}-PsCrtW + HpCrtZ + KDEL-T_{XPR2} | Example 3 |
| YL15 | YL00, URA3::P_{TEFin}-PsCrtW + HpCrtZ + SKL-T_{XPR2} | Example 3 |
| YL16 | YL14, LEU2::P_{TEFin}-PsCrtW + HpCrtZ + oleosin-T_{XPR2} | Example 3 |
| YL17 | YL16, TRP1::P_{TEFin}-PsCrtW + HpCrtZ + SKL-T_{XPR2} | Example 3 |

TABLE 3
Primers used in Example 3.
Individual enzymes

| | |
|---|---|
| BrCrtW-F | ATGAACCAGATGCATAGCACTTAAGACTCGCCTCGCCACA |
| | (SEQ ID NO: 7) |
| BrCrtW-R | ACACAAGACATATCTACAGCATGACCGCCGCTGTGGCTGA |
| | (SEQ ID NO: 8) |
| HpCrtW-F | ATGAACCAGATGCATAGCACTTAGGCCAGAGCGGGGACCA |
| | (SEQ ID NO: 9) |
| HpCrtW-R | ACACAAGACATATCTACAGCATGCACGTGGCCTCTGCTCT |
| | (SEQ ID NO: 10) |
| PsCrtW-F | ATGAACCAGATGCATAGCACTTAGGCGGTGTCGCCCTTAG |
| | (SEQ ID NO: 11) |
| PsCrtW-R | ACACAAGACATATCTACAGCATGTCTGCCCACGCTCTGCC |
| | (SEQ ID NO: 12) |
| BrCrtZ-F | TTTTGCAGTACTAACCGCAGGCCTGGCTGACCTGGATCGC |
| | (SEQ ID NO: 13) |
| BrCrtZ-R | GCAAGACCGGCAACGTGGGGTTAGGCGCCAGAAGAAGAGG |
| | (SEQ ID NO: 14) |
| HpCrtZ-F | TTTTGCAGTACTAACCGCAGCTGTCTAAGCTGCAGTCTAT |
| | (SEQ ID NO: 15) |
| HpCrtZ-R | GCAAGACCGGCAACGTGGGGTTATCGCTTAGACCAGTCCA |
| | (SEQ ID NO: 16) |
| PaCrtZ-F | TTTTGCAGTACTAACCGCAGCTGTGGATCTGGAACGCCCT |
| | (SEQ ID NO: 17) |
| PaCrtZ-R | GCAAGACCGGCAACGTGGGGTTACTTGCCAGAGGCGGGCT |
| | (SEQ ID NO: 18) |

Fusion enzymes

| | |
|---|---|
| HpCrtZ + PsCrtW-F1 | TTTTGCAGTACTAACCGCAGCTGTCTAAGCTGCAGTCTAT |
| | (SEQ ID NO: 15) |
| HpCrtZ + PsCrtW-R1 | CGGGTCCGCCAGAGCCTCCGCCGCCTCGCTTAGACCAGTCCA |
| | GCT (SEQ ID NO: 19) |
| HpCrtZ + PsCrtW-F2 | CGGAGGCTCTGGCGGACCCGGCTCTATGTCTGCCCACGCTCT |
| | GCC (SEQ ID NO: 20) |
| HpCrtZ + PsCrtW-R2 | GCAAGACCGGCAACGTGGGGTTAGGCGGTGTCGCCCTTAG |
| | (SEQ ID NO: 21) |
| PsCrtW + HpCrtZ-F1 | TTTTGCAGTACTAACCGCAGTCTGCCCACGCTCTGCCCAA |
| | (SEQ ID NO: 22) |
| PsCrtW + HpCrtZ-R1 | CGGGTCCGCCAGAGCCTCCGCCGCCGGCGGTGTCGCCCTTAG |
| | TTC (SEQ ID NO: 23) |
| PsCrtW + HpCrtZ-F2 | CGGAGGCTCTGGCGGACCCGGCTCTATGCTGTCTAAGCTGCA |
| | GTC (SEQ ID NO: 24) |
| PsCrtW + HpCrtZ-R2 | GCAAGACCGGCAACGTGGGGTTATCGCTTAGACCAGTCCA |
| | (SEQ ID NO: 16) |

Enzymes organelles targeting

| | |
|---|---|
| PsCrtW + HpCrtZ + | TTTTGCAGTACTAACCGCAGTCTGCCCACGCTCTGCCCAA |
| oleosin-F1 | (SEQ ID NO: 22) |
| PsCrtW + HpCrtZ + | CGGGTCCGCCAGAGCCTCCGCCGCCTCGCTTAGACCAGTCCA |
| oleosin-R1 | GCT (SEQ ID NO: 19) |
| PsCrtW + HpCrtZ + | CGGAGGCTCTGGCGGACCCGGCTCTATGGCCGACCGAGACCG |
| oleosin-F2 | AAG (SEQ ID NO: 25) |
| PsCrtW + HpCrtZ + | GCAAGACCGGCAACGTGGGGTTAGGAGGAAGCTCGACCAC |
| oleosin-R2 | (SEQ ID NO: 26) |
| PsCrtW + HpCrtZ + | TTTTGCAGTACTAACCGCAGTCTGCCCACGCTCTGCCCAA |
| KDEL-F | (SEQ ID NO: 22) |
| PsCrtW + HpCrtZ + | GCAAGACCGGCAACGTGGGGTTACAGCTCGTCCTTTCGCTTA |
| KDEL-R | GACCAGTCCAGCTCG (SEQ ID NO: 27) |
| PsCrtW + HpCrtZ + | TTTTGCAGTACTAACCGCAGTCTGCCCACGCTCTGCCCAA |
| SKL-F | (SEQ ID NO: 22) |
| PsCrtW + HpCrtZ + | GCAAGACCGGCAACGTGGGGTTACAGCTTGGATCGCTTAGAC |
| SKL-R | CAGTCCAGCTCG (SEQ ID NO: 28) |

TABLE 4
Codon-optimized sequence of oleosin used in
Example 3.

ATGGCCGACCGAGACCGAAGCGGCATCTACGGCGGCGCCCACGCTACCT
ACGGCCAGCAGCAGCAGCAGGGCGGCGGTGGTCGACCTATGGGCGAGCA
GGTCAAGGGCATGCTGCACGACAAGGGCCCCACTGCCTCCCAGGCCCTC
ACCGTTGCCACCCTGTTCCCCCTGGGCGGCCTCCTTCTCGTGCTTTCCG
GTCTTGCCCTGACTGCCTCCGTGGTCGGCCTGGCTGTCGCCACTCCCGT
CTTCCTCATCTTCTCCCCCGTCCTCGTCCCCGCCGCCCTTCTTATCGGC
ACCGCCGTCATGGGCTTCCTGACCTCTGGCGCCCTGGGCCTCGGCGGTC
TTTCCTCTCTCACCTGCCTTGCCAACACCGCCCGACAGGCCTTTCAGCG
AACCCCCGACTACGTCGAGGAGGCCCACCGACGAATGGCCGAGGCCGCT
GCTCACGCCGGTCACAAGACCGCCCAGGCCGGACAGGCCATCCAGGGAC
GAGCCCAGGAGGCCGGTGCCGGTGGTGGTGCTGGTGCTGGAGCCGGAGG
CGGTGGTCGAGCTTCCTCCTAA (SEQ ID NO: 29)

TABLE 5
The concentration of carotenoid involved in astaxanthin pathway.

| | β- | | | 3′- | | |
|---|---|---|---|---|---|---|
| | carotene | Echinenone | Canthaxanthin | Hydroxyechinenone | Zeaxanthin | Astaxanthin |
| Strains | (mg/L) | (mg/L)* | (mg/L)* | (mg/L)* | (mg/L)* | (mg/L) |
| YL02 | 502.5 | 33.9 | 56.6 | 2.2 | 23.0 | 9.9 |
| YL10 | 902.1 | 28.6 | 3.1 | 50.6 | 10.5 | 22.8 |
| YL11 | 788.8 | 28.3 | 5.2 | 56.7 | 12.4 | 28.9 |
| YL13 | 430.1 | 26.4 | 4.0 | 53.7 | 22.9 | 46.8 |
| YL14 | 574.4 | 28.9 | 11.6 | 34.3 | 32.8 | 53.2 |
| YL15 | 597.7 | 19.6 | 2.1 | 44 | 22.3 | 58.7 |
| YL16 | 161.1 | 6.8 | 3.2 | 20.5 | 12.6 | 115.1 |
| YL17 | 115.6 | 3.1 | 1.9 | 17.9 | 15.7 | 139.4 |

*Due to a lack of standard compounds, these intermediates were calculated as astaxanthin equivalence.

### Example 5. Removal of Lycopene Substrate Inhibition Enables High Carotenoid Productivity in Yarrowia lipolytica

Substrate inhibition of enzymes can be a major obstacle to the production of valuable chemicals in engineered microorganisms. Substrate inhibition of lycopene cyclase was identified as the main limitation in carotenoid biosynthesis in Yarrowia lipolytica. To overcome this bottleneck, two independent approaches were used. Structure-guided protein engineering yielded a variant, Y27R, characterized by complete loss of substrate inhibition without reduction of enzymatic activity. Alternatively, establishing a geranylgeranyl pyrophosphate synthase-mediated flux flow restrictor also prevented the onset of substrate inhibition by diverting metabolic flux away from the inhibitory metabolite while maintaining sufficient flux towards product formation. Both approaches were very effective, resulting in high levels of near-exclusive β-carotene production. Ultimately, strains capable of producing 39.5 g/L β-carotene at a productivity of 0.165 g/L/h in bioreactor fermentations (a 1441-fold improvement over the initial strain) were constructed. The findings described herein provide effective approaches for removing substrate inhibition in engineering pathways for efficient synthesis of natural products.

Engineering microbes for the production of valuable chemical products is an attractive alternative to sourcing these compounds from nature or deriving them from petrochemicals by chemical synthesis (Chen and Nielsen., 2013; Li and Smolke, 2016; Liu and Nielsen, 2019). However, synthetic biology efforts to achieve economically viable and scalable titers and productivities are frequently hindered by undesirable regulatory mechanisms that modulate the activity of enzymes. Such mechanisms have evolved to mediate optimal cellular response to changing physiological conditions, but also represent a major obstacle in redirecting metabolic fluxes toward desired engineered metabolic pathways and away from native growth-optimizing ones. This problem is particularly evident in compounds that require long and complex synthesis pathways (e.g., isoprenoids), frequently giving rise to bottlenecks that may reduce cell fitness and pathway productivity (Wu et al., 2016; Keasling et al., 2010). As such, it is imperative to develop methods that allow us to circumvent the effect of enzyme inhibition in constructing robust strains with high productivity.

Substrate inhibition represents one such enzyme-level regulation deployed in cells to help optimize cellular economy and maximize growth in response to temporal variations of the environment (Reed et al., 2010). Moreover, such mechanism is often used to design therapies for various diseases (Boutin et al., 2005; Belzer et al., 2013). However, it is undesirable in industrial applications of microbes mediated by enzymatic reactions for product synthesis. Enzyme inhibition is typically triggered when substrate concentration exceeds a certain threshold, thus preventing the catalytic conversion of the substrate and limiting the flux through the desired pathway. As such, substrate inhibition is particularly detrimental to the synthesis of end-products of interest when present in the middle of a metabolic pathway, which in turn causes intermediates accumulation, pathway disruption and alteration in the profile of products formed. Although several methods have been explored to address this limitation, such as enzyme immobilization (Singh et al., 2013; Matwo et al., 2004), two-phase partitioning bioreactor systems (Daugulis et al., 2011; Gao et al., 2009; Nielsen et al., 2009), batch substrate-feeding strategy (Kim et al., 2007), and protein engineering (Chen et al., 2014; Shang et al., 2020), most of these solutions are limited to systems where the inhibition is posed by the starting substrate, and difficult to apply in the context of microbial engineering for chemical production.

Using the oleaginous yeast Yarrowia lipolytica for carotenoid overproduction, two independent strategies are demonstrated that nearly completely circumvent substrate inhibition. First, the enzyme lycopene cyclase was identified as the bottleneck in the synthesis of carotenoids due to its strong substrate inhibition by lycopene. This resulted in not only low titers of β-carotene, but also large amounts of accompanying lycopene as byproduct. In light of this, the first strategy was to use a structure-guided protein design, coupled with phylogenetic information, to generate protein variants with reduced inhibition. Of the 50 variants generated, a single mutation Y27R was identified that completely abolished substrate inhibition without reducing enzyme activity, resulting in a remarkable increase of β-carotene production and 98% selectivity (% product vs sum of all carotenoids). Alternatively, in the second approach, similar titers and selectivity of β-carotene were obtained by reducing the carbon flow through the carotenoid pathway and thus preventing inhibitory metabolite accumulation to inhibitory levels, contrary to the traditional paradigms of pathway engineering. This was achieved by establishing a geranylgeranyl pyrophosphate synthase (GGPPS)-mediated metabolic flow restrictor that regulated the substrate lycopene formation rate, thereby effectively alleviating substrate inhibition. While this approach reduces flux through the pathway of interest, the gains from suppressing substrate levels and thus maintaining high enzymatic activity overcompensated for any losses in productivity suffered from the flux diversion. Using the methods outlined above, along with careful partitioning of cellular resources dedicated to carotenoid synthesis versus storage, a strain capable of producing 39.5 g/L β-carotene (98% selectivity) with a 0.165 g/L/h volumetric productivity in bioreactor fermentations was ultimately established. Moreover, by deliberately exploiting the substrate inhibition effect, the product profile was shifted towards lycopene instead, achieving lycopene titers of 17.6 g/L and productivities of 0.073 g/L/h.

### Substrate Inhibition of Lycopene Cyclase Limits Carotenoid Synthesis

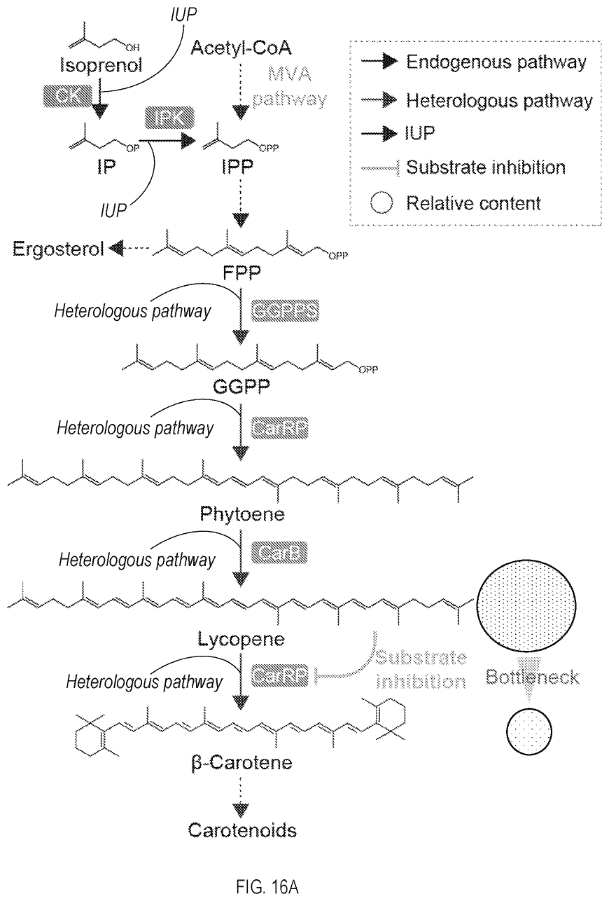

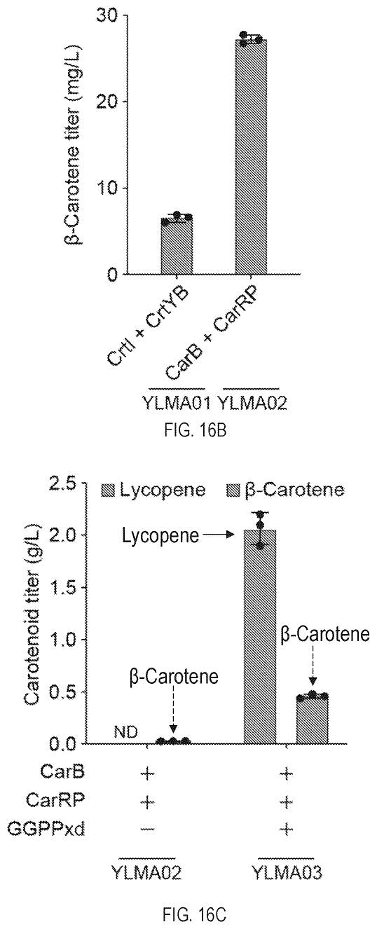

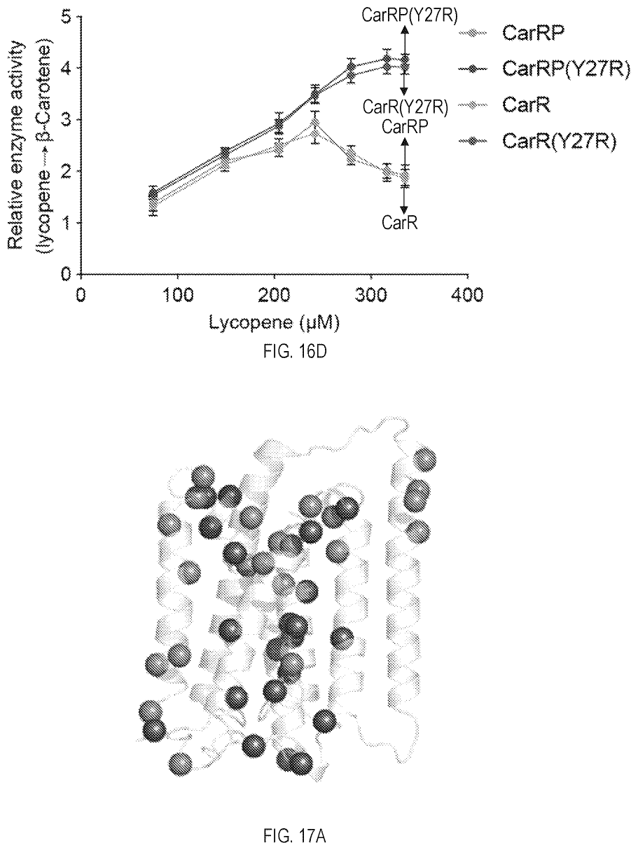

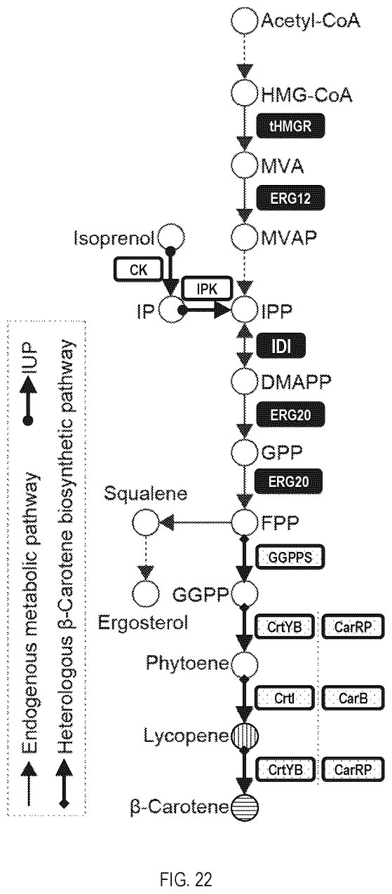

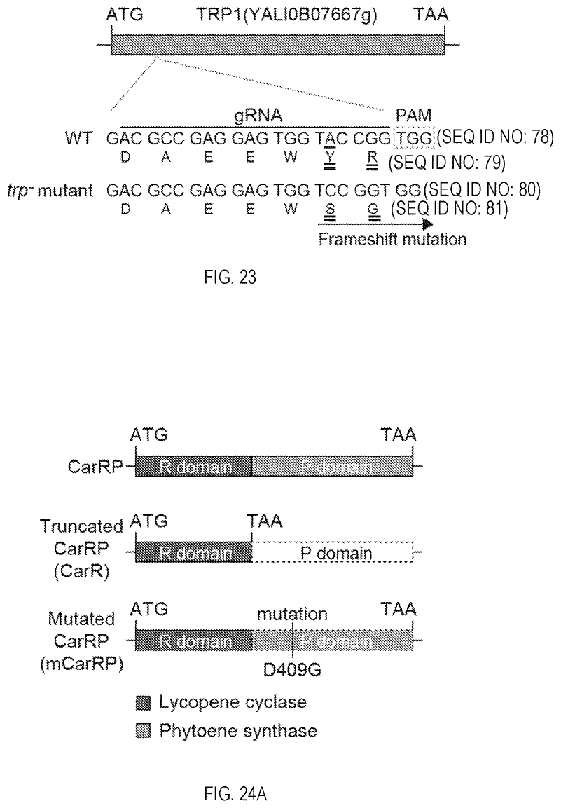

Synthesis of β-carotene in Y. lipolytica requires heterologous expression of three genes encoding the enzymes phytoene synthase, phytoene dehydrogenase, and lycopene cyclase (FIG. 16A and FIG. 22). Additionally, geranylgeranyl diphosphate synthase (GGPPS) should also be considered as it controls the flux directed towards carotenoid instead of sterol synthesis (FIG. 16A and FIG. 22). The relevant genes were sourced from the eukaryotic organisms, Xanthophyllomyces dendrorhous and Mucor circinelloides, for expression. Since Y. lipolytica already harbors a native copy of GGPPS, introducing gene expression cassettes encoding phytoene dehydrogenase and the bi-functional phytoene synthase/lycopene cyclase from X. dendrorhous (CrtI and CrtYB, respectively) was the first step (Verdoes et al., 1999; Verdoes, Krubasik et al., 1999) or M. circinelloides (CarB and CarRP, respectively) (Velayoes, Blasco et al., 2000; Velayos, Eslava et al., 2000) into the Y. lipolytica po1f strain with TRP1 disruption (po1f-T) (FIG. 23 and Table 6). Strain YLMA02, which expressed enzymes from M. circinelloides, produced 4.12-fold more β-carotene (27.4 mg/L) than strain YLMA01, which expressed enzymes from X. dendrorhous (FIG. 16B). Thus, the CarB/CarRP pair was used in all further studies.

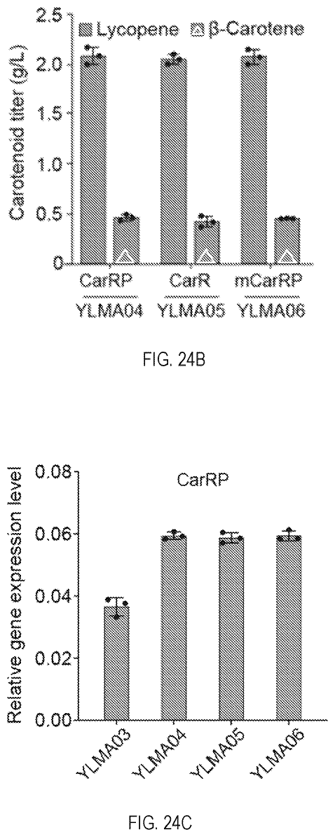

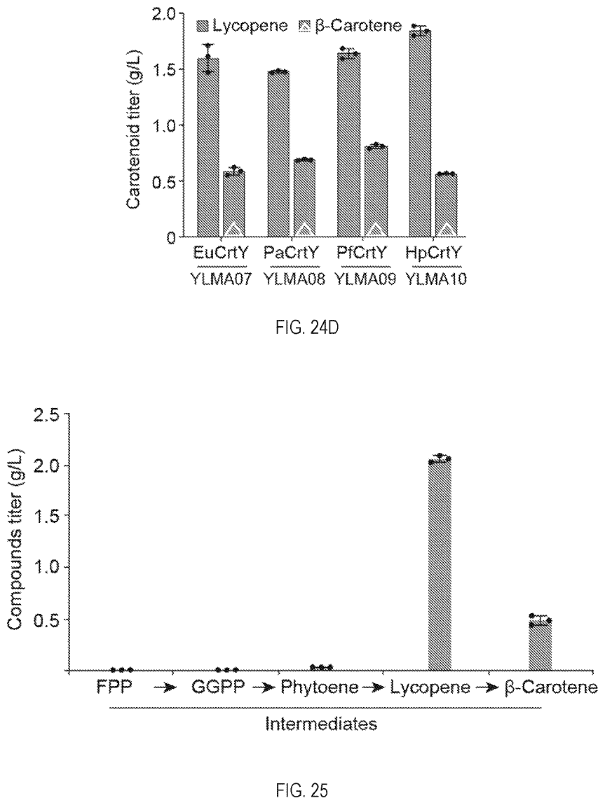

Although synthesis of β-carotene was observed in YLMA02, the titers were very low, prompting us to investigate the GGPPS step as the next target. Introduction of an additional copy of GGPPS from X. dendrorhous (GGPPxd) into strain YLMA02 significantly increased the titers of β-carotene to 0.48 g/L (FIG. 16C). However, this was also accompanied by a large increase in lycopene accumulation (FIG. 16C), which suggested that the cyclization from lycopene to β-carotene was a pathway bottleneck. Correspondingly, the expression of lycopene cyclase was increased by increasing its gene copy number. Moreover, since cyclase activity is conferred by the R domain of the bifunctional enzyme CarRP (Velayos, Eslava et al., 2000), a modified version of the protein with its P domain either deleted or mutated was also introduced (FIG. 24A), such that it served as a dedicated cyclase. None of these efforts succeeded in improving β-carotene titers (FIG. 24B), despite the higher mRNA level was observed (FIG. 24C). Next, the effect of introducing lycopene cyclases (EuCrtY, PaCrtY, PfCrtY and HpCrtY) from four other organisms was examined (Table 6), which led to modest improvements in β-carotene production (FIG. 24D). However, the issue of lycopene accumulation remained largely unaddressed, suggesting that the overexpression of lycopene cyclase, regardless of its origin, was not an effective strategy. It was very possibly that the protein level of lycopene cyclase was not the limitation in this study.

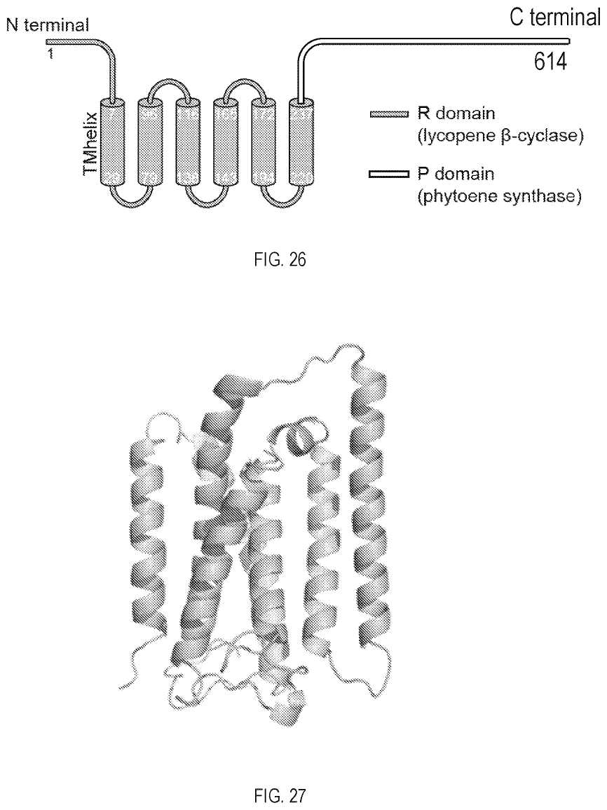

Since lycopene was the only aggregating precursor (FIG. 25) and adding additional copies of various lycopene cyclases did not circumvent the issue, it was hypothesized that the activity of lycopene cyclase was inhibited by excess lycopene through substrate inhibition (FIG. 16A). To test this hypothesis, possible correlations between lycopene cyclase activity and lycopene concentration were examined using an in vitro enzymatic assay that employed a yeast microsomal system, as CarRP is predicted to be a membrane protein with six transmembrane helices (FIG. 26). The results showed that lycopene cyclase activity was bi-phasic with respect to lycopene concentration: enzymatic activity increased with lycopene concentration initially, then decreased once lycopene reached higher concentrations (FIG. 16D). This supported the hypothesis that lycopene cyclase was substrate-inhibited, thus creating a major bottleneck in carotenoid biosynthesis.

### Structure-Guided Protein Engineering Completely Removes Substrate Inhibition

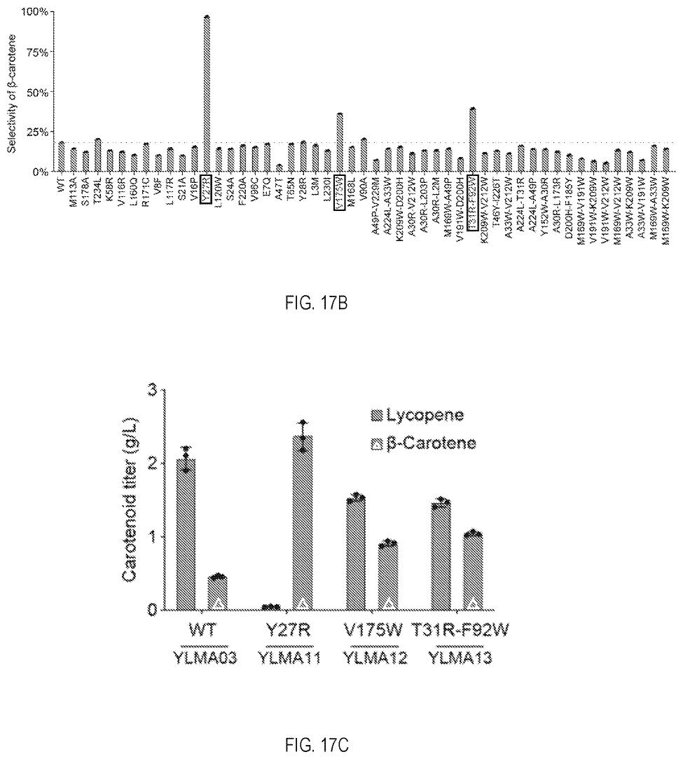

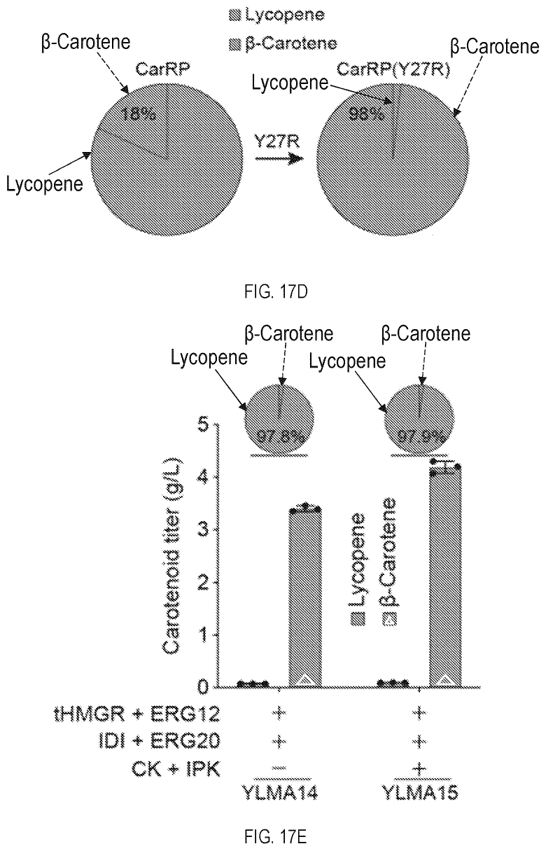

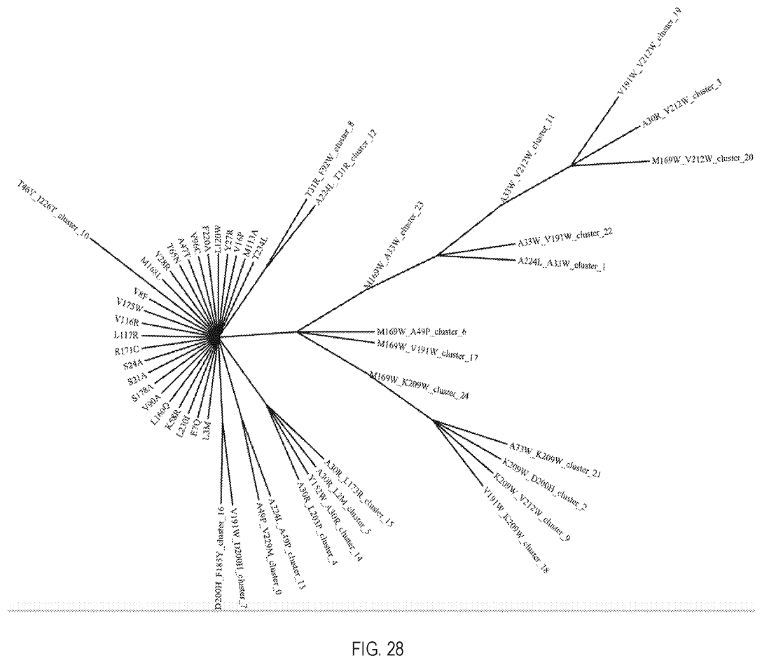

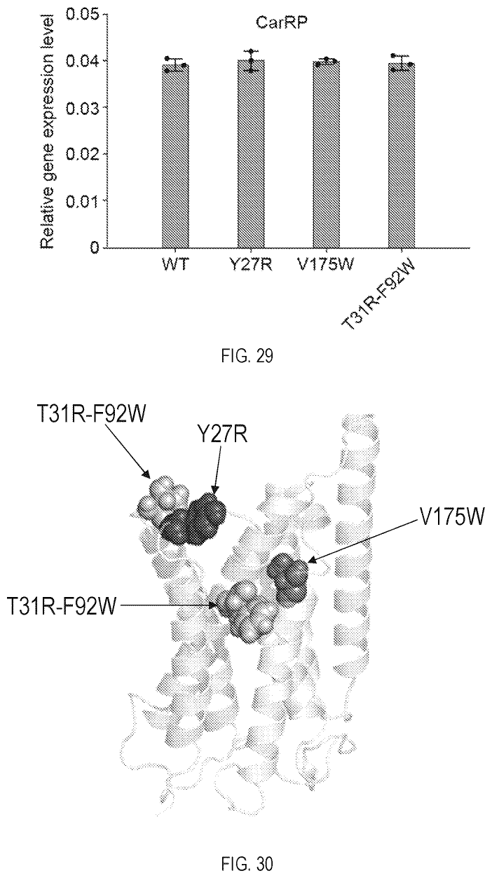

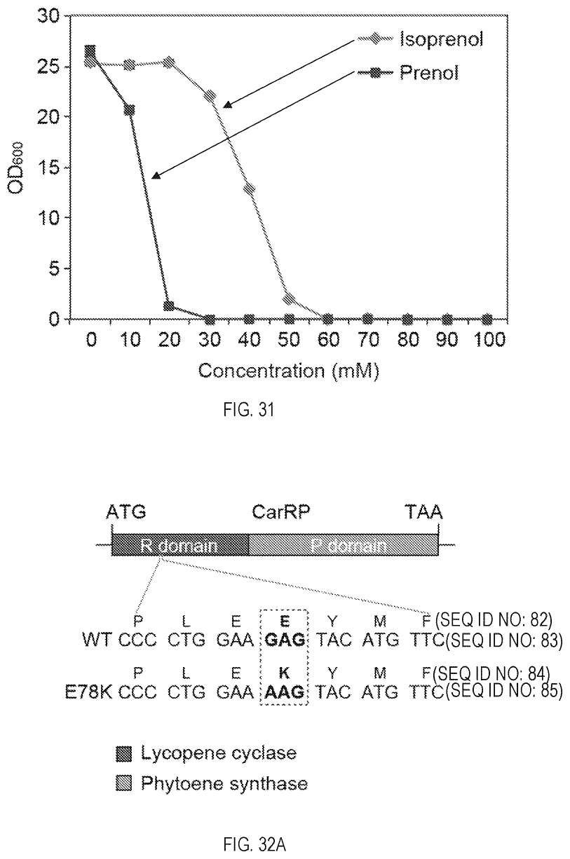

Removing the substrate inhibition effect of lycopene cyclase through protein engineering was evaluated next. As its crystal structure was unavailable, the Transform-restrained Rosetta (TrRosetta) platform (Yang et al., 2020) was used to create a computational model of the R domain (lycopene cyclase) of CarRP (FIG. 27). Evolutionary information was employed by generating a Position Specific Scoring Matrix (PSSM) from multiple sequence alignment to identify positions that could be mutated to deconvolute the areas of the enzyme that impacted substrate inhibition. Single and double amino acid substitutions were created based on the PSSM information and clustered to ensure maximum spread of the tested variants (FIG. 28). The variants were clustered using PAM30 to compute distances between sequences and agglomerative clustering to subdivide the sequences, maximizing information obtained during the initial screen. Through this method, \a set of 50 candidates were generated with mutations spread throughout the enzyme (FIG. 17A). The selectivity for β-carotene were obtained for each of the variants (FIG. 17B). Among them, 3 variants, Y27R, V175W, and T31R-F92W, displayed significantly increased β-carotene selectivity as well as improved production metrics (FIG. 17B-17C) without affecting gene expression (FIG. 29), suggesting alleviation of the substrate inhibition effect. The substitutions in all three variants were located in a specific part of the enzyme (FIG. 30), with Y27R being the most pronounced for loss of inhibition. The variant Y27R demonstrated a complete loss of substrate inhibition without reduction in enzyme activity (FIG. 16D), and yielded a titer of 2.38 g/L of β-carotene (FIG. 17C), along with a selectivity of 98% (compared to 18% of the wild type, FIG. 17D).

It was then investigated whether the β-carotene pathway containing variant Y27R was able to maintain its properties of minimal substrate inhibition in the presence of considerably higher precursor/substrate formation rates. To this end, four key enzymes were overexpressed, tHMGR, ERG12, IDI, and ERG20, of the Mevalonate (MVA) pathway (Ro et al., 2006; Westfall et al., 2012) (FIG. 22) in the β-carotene producing strain YLMA11 expressing Y27R (FIG. 17E). This resulted in an increased titer of 3.43 g/L β-carotene while maintaining the high selectivity of 97.8%. In addition, the synthetic Isopentenol Utilization Pathway (IUP) (Chatzivasileiou et al., 2019; Clomburg et al., 2019; Rico et al., 2019; Lund et al., 2019) was introduced through the expression of Choline Kinase (CK) and Isopentenyl Phosphate Kinase (IPK) (FIG. 22), resulting in an additional 23% increase in β-carotene production (4.22 g/L), without any loss in selectivity (YLMA15, FIG. 17E). These results demonstrate that the reconstituted non-substrate inhibition pathway functions efficiently in high isoprenoid flux strains, not affected by the intracellular levels of precursor/substrate in the cells.

### Managing Substrate Inhibition of Lycopene Cyclase Through a GGPPS-Mediated Flux Flow Restrictor

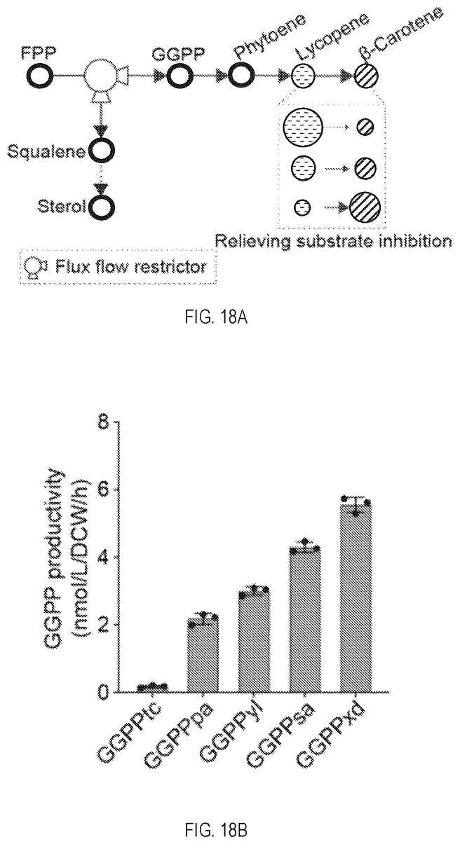

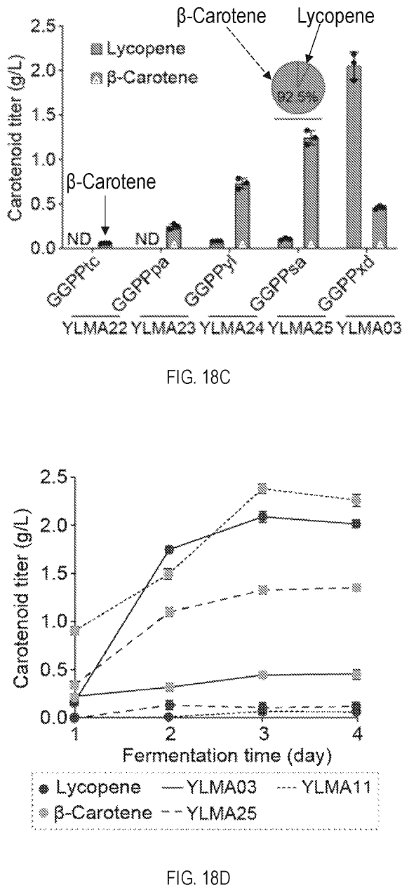

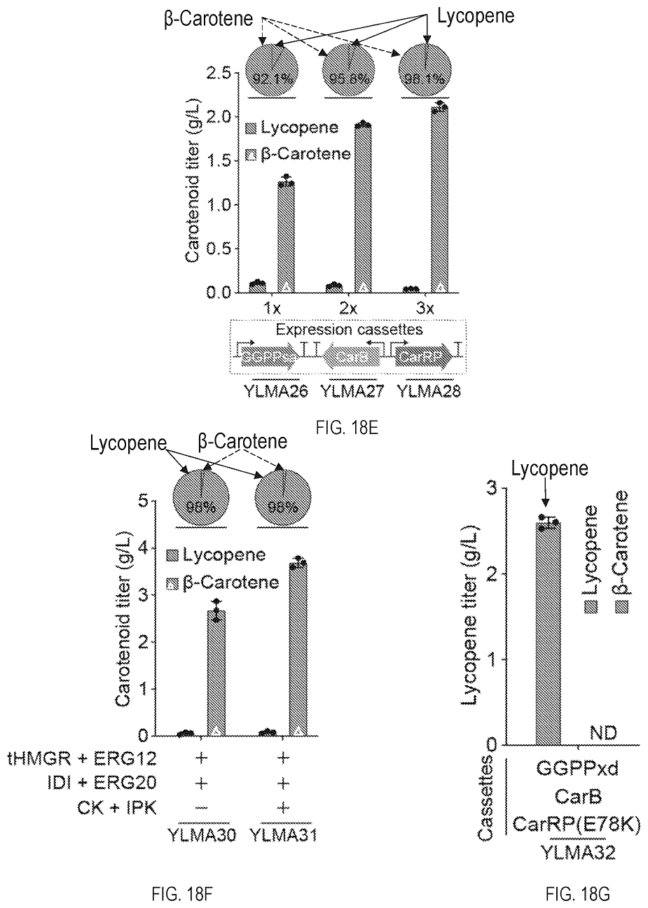

Other viable options were also explored that can eliminate the substrate inhibition without the need to modify lycopene cyclase. It was hypothesized that attenuating the formation rate of lycopene relative to its conversion rate could potentially lower the intracellular concentration of lycopene below the inhibitory level. However, this needs to be well-tuned to prevent an overall reduction of the production rate of end-product by attenuating too much the lycopene formation rate. To this end, the branching point at the FPP node was exploited to create a metabolic flow restrictor and regulate flux towards lycopene such as to maintain sub-inhibitory levels, yet high lycopene conversion into β-carotene (FIG. 18A). As such, GGPPS mutants with variable activity were searched by screening five different enzymes (Table 6) with diverging catalytic efficiencies measured by their in vivo GGPP synthesis rates (FIG. 18B). Relative to GGPPxd, the other four GGPPSs exhibited lower productivities (FIG. 18B), which should translate to lower lycopene synthesis flux. Upon introduction of these lower-activity GGPPSs into strain YLMA02 (base strain harboring wild-type CarRP), lycopene levels were reduced (FIG. 18C) and β-carotene production increased, reaching up to 1.26 g/L with 92.5% selectivity when GGPPsa from Sulfolobus acidocaldarius was used (FIG. 18C). This was further confirmed by the time courses of lycopene and β-carotene concentrations (FIG. 18D). Interestingly, although the expression of the attenuated GGPPsa (YLMA25) slightly lowered flux committed to carotenoid synthesis, it circumvented any substrate inhibition and enabled an overall balanced pathway that directs all carbon flux towards β-carotene formation, allowing the pathway to behave similar to the variant with Y27R (YLMA11, FIG. 18D). In contrast, strain YLMA03 expressing the very efficient GGPPxd caused the buildup of lycopene at an overly rapid rate, triggering substrate inhibition and preventing its conversion into β-carotene (FIG. 18D). These results indicate that substrate inhibition could be effectively relieved by the GGPPS-mediated metabolic flow restrictor to regulate flux through both up- and downstream of lycopene for its optimal conversion into β-carotene.

In order to close the gap of β-carotene production between the two engineering strategies that mitigate substrate inhibition (FIG. 17C and FIG. 18C), the GGPPS-mediated flux flow restrictor-supported pathway was overexpressed by inserting additional copies (FIG. 18E). This modulation not only strengthened β-carotene biosynthesis up to 2.13 g/L, but also further maximized its selectivity (from 92.1% to 98.1%, FIG. 18E). Furthermore, similar to the previous results, overexpression of MVA and IUP promoted the production of β-carotene up to 3.72 g/L without affecting selectivity (FIG. 18F). This further underlined the fact that the pathway can operate at high efficiency once the substrate inhibition issue has been addressed.

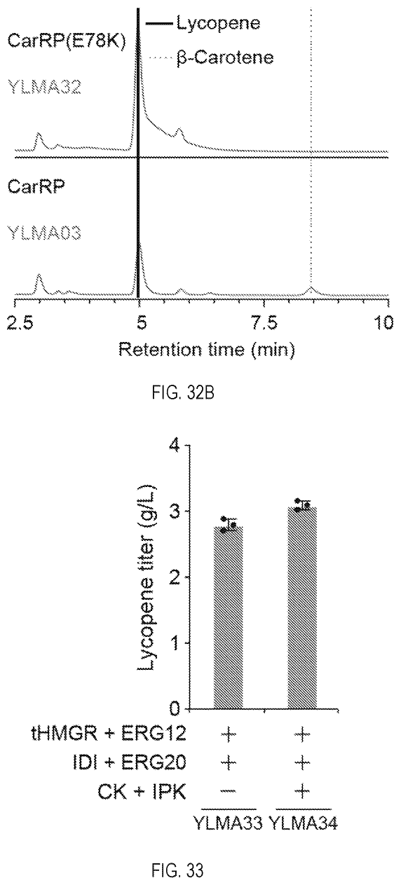

The product profile of β-carotene versus lycopene can be shifted by varying the in vivo GGPPS activity (FIGS. 18B-18C). The high activity of GGPPxd can also be taken advantage of to reconstitute a dedicated lycopene-producing strain. Combining this idea with a CarRP variant (E78K) (Velayos, Eslava, et al., 2000) (FIGS. 32A-32B), 2.62 g/L of lycopene with undetectable amounts of β-carotene were successfully produced (FIG. 18G). An additional increase in lycopene production was achieved by overexpressing the MVA pathway and introducing IUP, reaching titers of 3.09 g/L (YLMA34, FIG. 33).

### Partitioning Carbon Flux Between Isoprenoid and Lipid Synthesis to Enhance Intracellular Carotenoid Accumulation

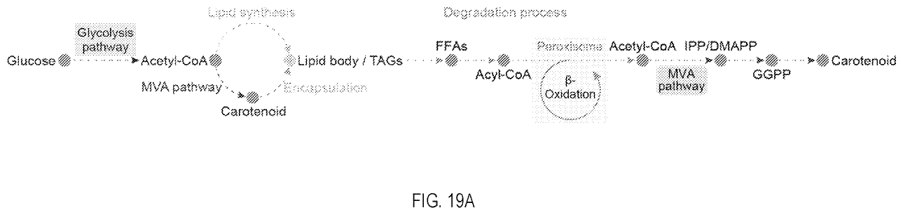

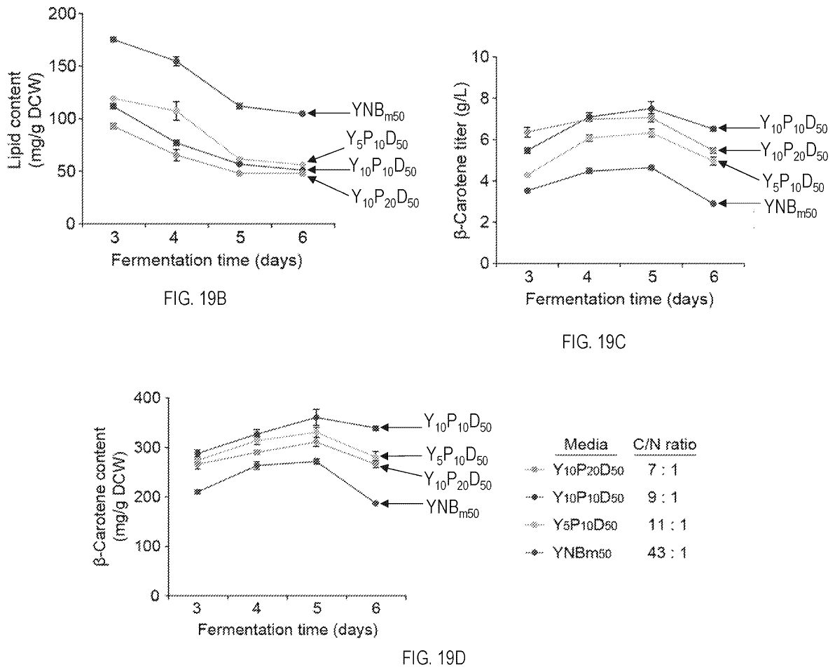

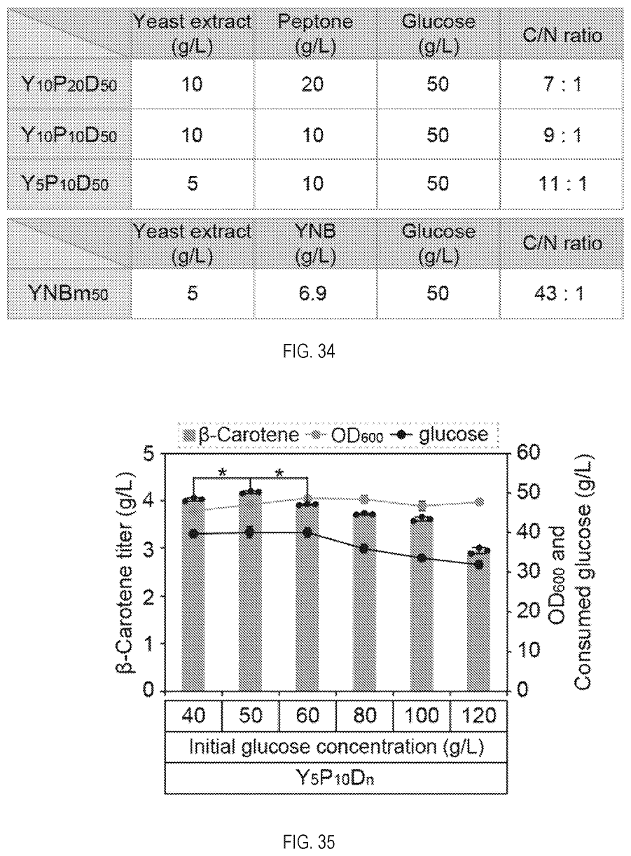

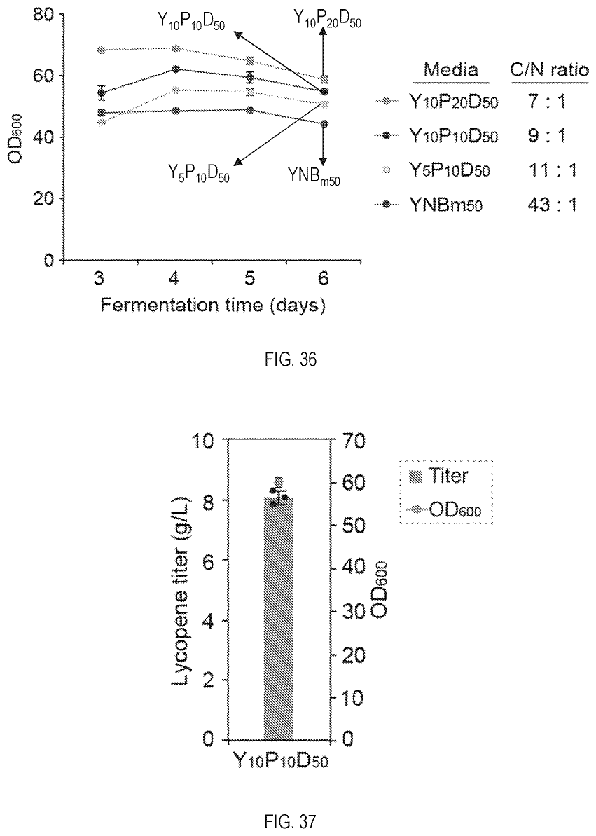

Lipid bodies in Y. lipolytica create hydrophobic pockets that facilitate lipophilic isoprenoid product sequestration and storage (Qiao et al., 2017). However, while increased triacylglycerol (TAG) supply would enhance isoprenoid storage (Ma et al., 2019; Larroude et al., 2018), this comes at the expense of acetyl-CoA, a common precursor for isoprenoid and lipid synthesis (FIG. 19A). Therefore, carbon flux needs to be optimally partitioned between lipid and isoprenoid synthesis, with the goal of ensuring sufficient supply of lipids to encapsulate the produced isoprenoid while not drawing too much acetyl-CoA away from the MVA pathway. To this end, it was set out to culture the β-carotene-producing strain YLMA15 in media with varying Carbon-to-Nitrogen (C/N) ratios (FIG. 34), as the capacity for lipid formation can be readily controlled by the C/N ratio of the culture media (Braunwald et al., 2013; Somashekar et al., 20000). The initial glucose concentration for all conditions was fixed at 50 g/L, which was determined to be the optimum for the strains (FIG. 35). It was found that with increasing C/N ratios, the lipid content of the cells increased monotonically (FIG. 19B), while the total biomass decreased (due to reduced nitrogen availability, FIG. 36). However, an optimal condition for β-carotene production was obtained in terms of both titer (7.5 g/L, FIG. 19C) and cellular content (360.8 mg/g DCW, FIG. 19D) by using Y₁₀P₁₀D₅₀ (10 g/L yeast extract, 10 g/L peptone and 50 g/L glucose) media with a C/N ratio of 9:1. Deviations from this optimum resulted in diminishing β-carotene levels, which was consistent with the hypothesis and highlighted the importance of optimally balancing carotenoid and lipid biosynthesis. The optimal Y₁₀P₁₀D₅₀ media was also applied to the lycopene-producing strain (YLMA34), where a concentration of 8.02 g/L lycopene was obtained after a 5-day fermentation (FIG. 37).

### Carotenoid Biosynthesis During Glucose-Depleted Stationary Phase is Supported by Cellular Lipid Degradation

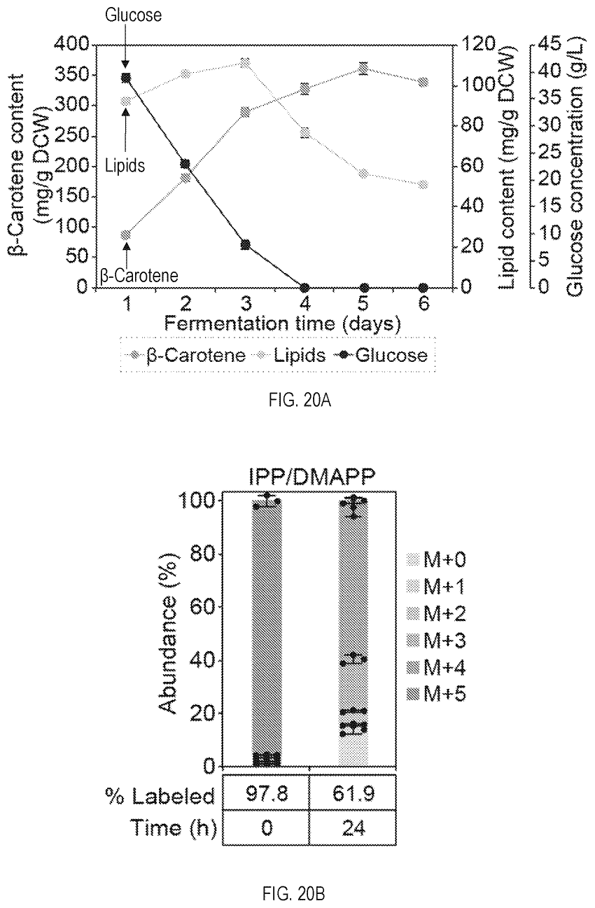

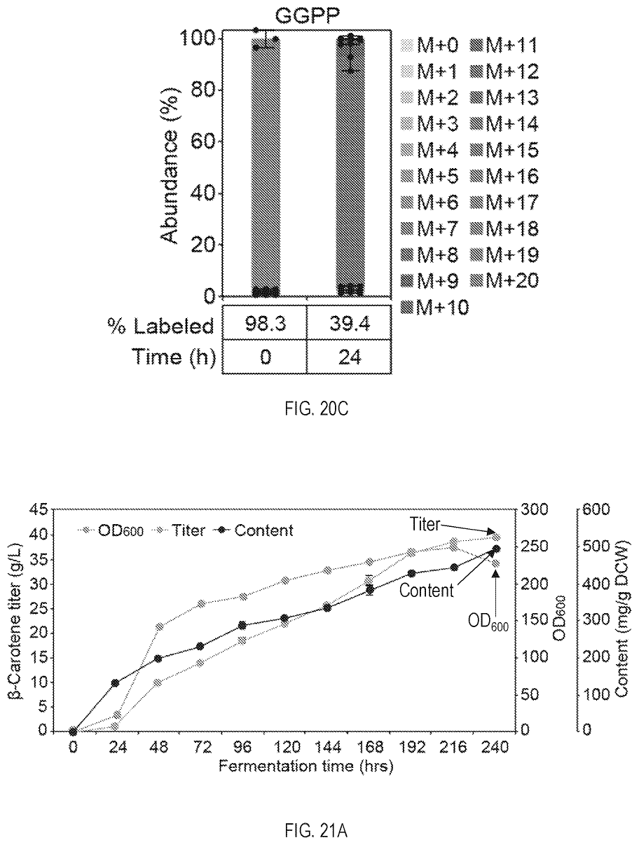

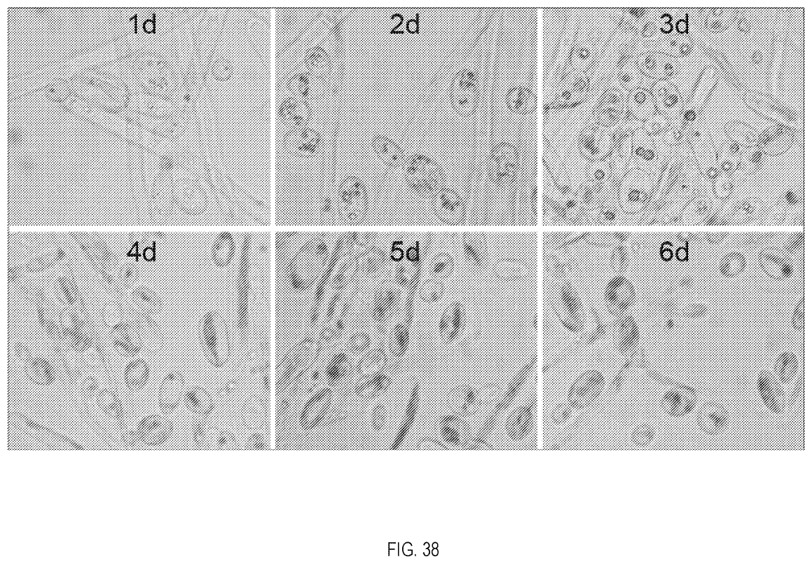

In the fermentation experiments with strain YLMA15 described herein, it was found that the content of β-carotene continued to rise during the stationary phase, even after glucose in the media had been depleted (FIG. 20A). It is likely that Y. lipolytica mobilized previously stored TAGs as an alternative carbon source to support carotenoid formation (Wang et al., 2020). To test this hypothesis, it was set out to characterize the cells along with their intracellular lipids both before and after glucose depletion. Throughout the fermentation process, lipid content increased initially, reaching a maximum at day 3, after which it rapidly decreased when glucose was fully consumed (FIG. 20A). Yet, despite the reduction in lipid content, β-carotene content continued to rise well past the point of glucose depletion (FIG. 20A), suggesting that TAGs were utilized to sustain metabolic activity and carotenoid synthesis. Microscopic visualization of the changes that occurred during fermentation was consistent with this hypothesis. When glucose was still present during the initial 3 days, lipid droplets within cells progressively agglomerated into lipid bodies that sequestered the produced β-carotene (FIG. 38). However, it was also evident that the lipid bodies were no longer visible during the later stages of fermentation due to TAG breakdown, which in turn caused the accumulated β-carotene to be more dispersed throughout the cell (FIG. 38).

Since TAG degradation occurs primarily through β-oxidation to generate acetyl-CoA (Xu et al., 2016) (FIG. 19A), it is likely that this provides the carbon backbone for β-carotene synthesis during the glucose-depleted stationary phase. To test this hypothesis, YLMA15 was cultured in YNB media with uniformly labeled [U-¹³C₆]glucose and natural abundance stearic acid. Upon the addition of stearic acid, which is catabolized through β-oxidation, large fractions of unlabeled IPP/DMAPP and GGPP were observed, the main precursors for β-carotene synthesis, within 24 h of cultivation (FIGS. 20B-20C). These results suggested that the acetyl-CoA formed through β-oxidation could indeed support MVA pathway and eventually contribute to β-carotene formation. Therefore, this is likely the mechanism used by the cells to convert TAGs into carotenoids during the glucose-depleted stationary phase.

### Bioreactor Cultivation Studies

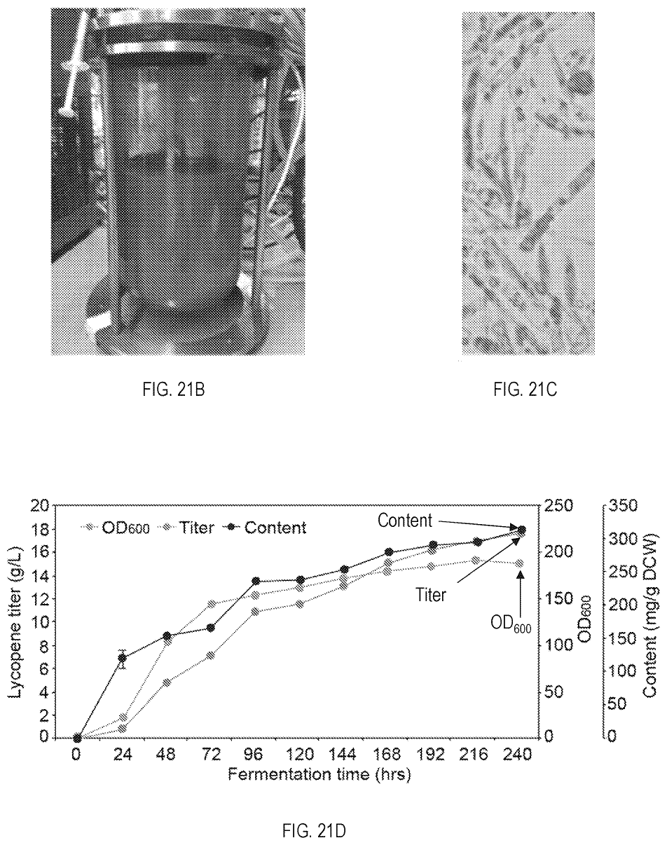

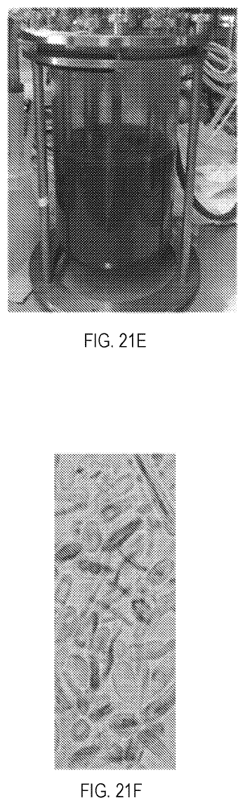

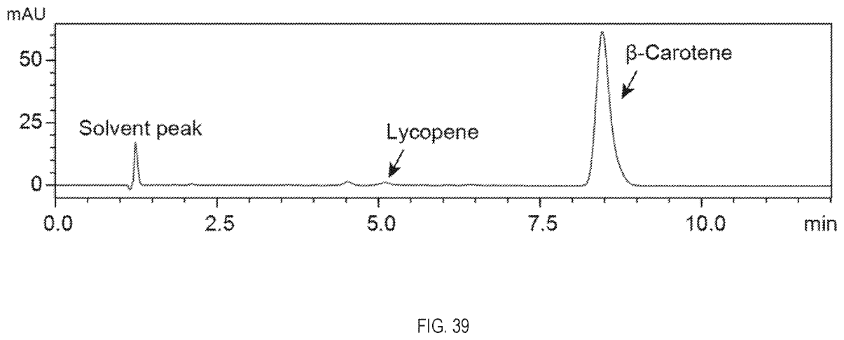

It was finally evaluated the performance of the constructed strains alleviated from substrate inhibition in 3-L fed-batch cultivations. After bioreactor optimizations, strain YLMA15 achieved a total β-carotene titer and content of 39.5 g/L and 494 mg/g DCW, respectively, with a productivity of 0.165 g/L/h (FIGS. 21A-21C). Likewise, bioreactor fermentation of the lycopene-producing strain YLMA34 yielded 17.6 g/L of lycopene (313 mg/g DCW) with a productivity of 0.073 g/L/h (FIGS. 21D-21F). Notably, no changes in the selectivity for β-carotene in strain YLMA15 during the scale-up process was observed (98% in fed-batch bioreactor, compared to 97.9% in shake flasks (FIG. 39)). These figures demonstrate the robustness of the engineering strategy in large culture volumes and high-cell density fermentation, with production metrics exceeding previous results (a summary of reported carotenoid production is provided in Table 8).

The present disclosure relates to demonstrating that lycopene cyclase undermines β-carotene production by substrate inhibition, a regulatory effect less reported in the context of microbial synthesis. Substrate inhibition of enzymes could be overcome through modification of the protein structure, a strategy that has been successfully applied to many enzymes (Shang et al., 2020). However, these efforts rely on readily available protein crystal structures, which is not the case for the lycopene cyclase investigated here. Although directed evolution is a powerful method of adapting enzymes to specific tasks (Reetz et al, 2013), it often requires high-throughput detection methods to screen large libraries. In addition, due to lack of crystal structure information, efficient structure-driven design heavily relies on both the quality of computational modeling and the accuracy to dock the substrate to its binding site. Here, combining structure and phylogenetic information, sharpened the search and allowed us to isolate a promising mutant by screening only 50 variants, with 3 of the variants exhibiting diminished or removed substrate inhibition. Furthermore, information from key amino acids could be iteratively fed back into the computational model to further optimize enzyme properties. To understand mechanistically what factor caused the removal of the substrate inhibition would require more thorough investigation that is beyond the scope of this study. The low number of protein variants designed and tested suggests that structure-guided approach coupled with phylogenetic information offers an effective strategy for protein engineering.

The degree of substrate inhibition can also be controlled by tuning the relative rates of up- and downstream pathways forming and consuming the inhibiting substrate. In the case of β-carotene synthesis, selecting GGPPS variants with lower activity reduced the flux through the carotenoid pathway. Yet, the resulting abolishment of substrate inhibition enabled all carotenoid flux to be diverted to β-carotene synthesis, as opposed to a combination of both lycopene and β-carotene. This led to an increased β-carotene production at high specificity (>98%) despite a lower GGPPS activity. On the other hand, substrate inhibition can also be deliberately exploited if lycopene is the desired product. In this case, a highly-efficient GGPPS can cause lycopene formation to outpace its depletion, leading to its accumulation, which then further amplifies the imbalance through substrate inhibition. Correspondingly, the product profile shifts drastically from β-carotene-rich to lycopene-rich. These findings illustrate that engineering proximal enzymes can have profound effects on pathway dynamics, providing a new paradigm for controlling metabolism.

Another important consideration in metabolic engineering is how heterologous pathways interact with the native ones. Designing pathways that are orthogonal to or have minimal impact on the native functions of an organism has been a focal point of many strain engineering efforts (Ro et al., 2006; Tan et al., 2016; Zhao et al., 2018; Brockman et al., 2015). It is well known that the lipophilic nature of carotenoids promotes their storage in the lipid bodies of the cells. Larroude et al. (Larroude et al., 2018) found that an engineered lipid overproducer strain was capable of producing more β-carotene with a titer of 6.5 g/L, while accompanied with the production of 42.6 g/L lipids. While providing compatible compartments for hydrophobic isoprenoid accumulation, the de novo formation of TAGs also consumes large amount of carbon source, resulting in limited acetyl-CoA flux into the MVA and product-forming pathway. Here, it was demonstrated that Y. lipolytica's native capacity for TAG accumulation is sufficient for carotenoid sequestration. By balancing the flux distribution between carotenoid and lipid synthesis through C/N ratio adjustments, a larger portion of the acetyl-CoA pool was preserved for carotenoid production, achieving higher titers and per-cell content.

### Example 6. Materials and Methods Related to Example 5

### Culture Conditions and Media

Escherichia coli DH5α cells were grown in Luria-Bertani (LB) media (BD bioscience) at 37° C. with constant shaking. Corresponding antibiotics (100 μg/mL ampicillin and 50 μg/mL kanamycin) were added for plasmid selection. All Yarrowia lipolytica strains were cultivated at 30° C. with shaking at 230 rpm. For Y. lipolytica, YPD media consisted of 10 g/L yeast extract (BD bioscience), 20 g/L peptone (BD bioscience), and 20 g/L glucose (Sigma-Aldrich) was used. Additionally, YNB media composed of 1.7 g/L yeast nitrogen base (YNB, VWR Life Science), 20 g/L glucose, 5 g/L ammonium sulfate (VWR Life Science), 15 g/L agar (BD bioscience), and 0.77 g/L appropriate complete supplement mixture without uracil, leucine, or tryptophan (Sunrise science products) was used for selecting transformed Y. lipolytica strains.

### Construction of Plasmids and Strains

E. coli DH5a was used for cloning and plasmid propagation. The Y. lipolytica po1f strain served as the base strain, and all derivatives and plasmids constructed in the present study are listed in Table 7. The primers used for plasmid construction are shown in Table 9. All restriction enzymes were purchased from New England Biolabs (NEB). PCR amplification was performed using Q5 high-fidelity DNA polymerase (NEB) or GoTaq DNA polymerase (Promega). PCR fragments were purified using the ZYMO Fragment Recovery Kit (ZYMO research). Plasmids were then constructed from the purified PCR fragments with Gibson Assembly kit (NEB), transformed into chemically competent E. coli cells by heat shock, and extracted using the QIAprep Spin Miniprep Kit (Qiagen). All procedures were performed according to the manufacturer instructions. All engineered Y. lipolytica strains were constructed by transforming linearized plasmids (Not1 digestion) using the lithium-acetate method. Recombinants were verified by PCR amplification from genomic DNA. The carotenoid biosynthetic genes evaluated in this study were all codon-optimized towards Y. lipolytica.

TRP1 Disruption in po1f Strain Using CRISPR-Cas9

For TRP1 disruption, the CRISPR-Cas9 plasmid (Schwartz et al., 2016) containing gRNA (ACGCCGAGGAGTGGTACCGG) (SEQ ID NO: 30) targeting the TRP1 (YALI0B07667g) gene of Y. lipolytica was transformed into strain po1f using Ura3 as the auxotrophic marker. The strain with tryptophan auxotrophy was obtained by selecting on YNB-Ura and YNB-Ura-Trp plates. After that, the positive clones were inoculated onto YPD plates and sub-cultured three times to lose the CRISPR-Cas9 plasmid, resulting in the po1f-T strain (ura3⁻, leu2⁻, trp1⁻).

### Auxotrophic Markers Curation by the Cre-loxP System

Plasmid pYLMA-Cre was transformed into target Y. lipolytica strains to rescue URA3, LEU2 and TRP1 markers. Transformants were selected on YPD agar plate supplemented with a final concentration of 250 mg/L hygromycin B (Sigma-Aldrich). After 2-3 days of cultivation, colonies were transferred onto a new YPD plate containing hygromycin B for 1 more day to allow for more successful marker deletions. Markers curation was confirmed by subculturing the colonies onto YNB-Ura, YNB-Leu, and YNB-Trp agar plates, respectively. Successful removal of all three markers will lead to a phenotype conferring uracil, leucine and tryptophan deficiency. Plasmid pYLMA-Cre in cells was then removed by incubating positive strains on YPD agar plates at 30° C. for 24 h, with 2-3 repeats.

### Shake Flask Fermentations

Single colonies of recombinant strains were picked from plate, inoculated into 2 mL YPD media, and cultivated overnight (16˜18 h) at 30° C. and 230 rpm. The culture was then transferred to a 50 mL shake flask containing 10 mL YPD media (initial OD₆₀₀=0.1), and cultivated at 30° C. with shaking at 230 rpm for 3˜5 days. When applicable, 30 mM isoprenol (Sigma-Aldrich) was added into YPD media when glucose of culture was nearly consumed.

### Bioreactor Fermentations

Fed-batch fermentations were performed in a 3 L bioreactor (New Brunswick Bioflo115 system). The initial fermentation was completed with 1 L medium containing 100 g/L glucose, 100 g/L peptone, and 50 g/L yeast extract. The temperature was maintained at 30° C. The dissolved oxygen was controlled at 20% with an agitation cascade of 250˜800 rpm. Air was sparged into fermenter at 2 vvm. The pH was maintained at 6.8 by feeding 5 M NaOH or 5 M HCL. Foam was prevented by the addition of antifoam 204 (Sigma-Aldrich). The fed-batch process was initiated after 48 h of cultivation with the 10×Y₁₀P₁₀D₅₀ media consisting of 100 g/L yeast extract, 100 g/L peptone and 500 g/L glucose. Once the media feeding starting, the agitation and aeration was changed and held constantly at 600 rpm and 0.3 vvm, respectively. Samples were taken every 24 h to measure OD₆₀₀, glucose concentration, and carotenoid titer.

### Quantification of Residual Glucose in Media

500 μL sample was extracted from the culture for residual glucose quantification. The cells were centrifuged at 12,000 rpm for 5 min, and then the supernatant was filtered through 0.2 μm syringe filters prior to injection into an Agilent technologies 1260 High-Performance Liquid Chromatography (HPLC) equipped with a refractive index detector. A Bio-Rad HPX-87H column was used for separation with 14 mM sulfuric acid as the mobile phase flowing at a rate of 0.7 mL/min. The injection volume was 10 L. The column temperature was 50° C.

### Lipid Extraction and Quantification

The fatty acids synthesized by Y. lipolytica including palmitate (C16:0), palmitoleate (C16:1), stearate (C18:0), oleate (C18:1) and linoleate (C18:2) were quantified using a Gas Chromatography coupled to a Flame Ionization Detector (GC-FID). 0.1˜1 mL cell culture was extracted from each bioreactor such that the sample contained approximately 1 mg biomass. A centrifugation step at 16,000 g for 10 min was performed and the supernatant discarded. 0.5 mL of a 0.5 M sodium hydroxide-methanol solution (20 g/L sodium hydroxide in anhydrous methanol) was mixed with the cell pellets, followed by the addition of 100 L internal standards containing 2 mg/mL methyl tridecanoate (Sigma-Aldrich) and 2 mg/mL glyceryl triheptadecanoate (Sigma-Aldrich) dissolved in hexane. Methyl tridecanoate was used for volume loss correction during sample preparation and glyceryl triheptadecanoate was used for transesterification efficiency correction. The samples were vortexed for 1 h to allow for the transesterification of lipids to fatty acid methyl esters (FAMEs). Afterwards, 40 μL of 98% sulphuric acid (Sigma-Aldrich) was added to neutralize the pH. The FAMEs were then extracted through the addition of 0.5 mL hexane followed by vortexing for 30 min. Centrifugation at 12,000 g for 1 min was then performed to remove cellular debris and the top hexane layer was extracted for analysis. Separation of the FAME species was achieved on an Agilent HP-INNOWax capillary column. The injection volume was 1 μL, split ratio was 10, and injection temperature was 260° C. The column was held at a constant temperature of 200° C. and helium was used as the carrier gas with a flow rate of 1.5 mL/min. The FID was set at a temperature of 260° C. with the flow rates of helium make up gas, hydrogen, and air at 25 mL/min, 30 mL/min, and 300 mL/min, respectively.

### Intracellular Metabolites Extraction and Quantification

To extract intracellular metabolites (e.g. IPP/DMAPP, and GGPP), 1 mL culture was filtered through a 25-mm 0.2 μm nylon filter using vacuum filtration. The cells were washed immediately with 2 mL of water preheated to 30° C., and the filter was submerged in ice-cold extraction buffer (40% methanol+40% acetonitrile+20% water). After incubation at −20° C. for 20 min, the extract solution was centrifuged at 16,000 rpm for 10 min, and the supernatant was transferred to a new tube and dried. The sample was resuspended with 50 μL water, and then centrifuged at 16,000 rpm for 10 min. Metabolites in supernatant were quantified by liquid chromatography-tandem mass spectrometry (LC-MS/MS) comprised of an Agilent 1100 series LC system and an AB Sciex API-4000 MS. 10 μL sample was injected and separation was achieved on a Waters XBridge C-18 column with a mobile phase consisting of solution A (0.1% tributylamine, 0.12% acetic acid, 0.5% 5M NH₄OH in water, v/v) and solution B (100% acetonitrile). The flow rate was 0.3 mL/min and the following gradients were used: 0-5 min, 0% B; 5-20 min, 0˜65% B; 20-25 min, 65% B; 25-30 min, 100% B; 30-35 min, 100% B; 35-36 min 100˜0% B, 0% B until 45 min. The analytes were then compared to standard curves generated using chemical standards purchased from Sigma-Aldrich and Cayman Chemicals.

### Labeling Experiments

Strains used in labeling studies were revived in YNB media with [U-¹³C]glucose as the sole carbon source. They were then subcultured in the same media and grown until early stationary phase at 30° C. Samples were taken before the start of the pulse addition of an extra carbon source using the same intercellular metabolite extraction method. Afterwards, 10 mM stearic acid was added to the corresponding cultures, and measurements of metabolite isotopic enrichments were taken at different time points. The optical densities associated with each sample were also recorded. IPP/DMAPP, and GGPP were quantified by LC-MS/MS as previously stated. All MS data from labeling experiments were corrected for natural abundance using IsoCor (Millard et al., 2012).

### Extraction of Carotenoids

Carotenoid extraction was performed as described (Asker et al., 2017) with the following modification. Briefly, 100 μL culture was centrifuged for 1 min at 16,000 g, and cell pellets were suspended in 900 μL dimethyl sulfoxide (DMSO, Sigma-Aldrich) prior to heating at 50° C. for 1 h until the cells bleached in a water bath. The DMSO extracts were briefly mixed with 450 μL of methanol and centrifuged at 16,000 g for 5 min. The resultant supernatants were transferred into 96-well assay plates or glass vials for carotenoid analysis and quantification.

### Analysis and Quantification of Carotenoids

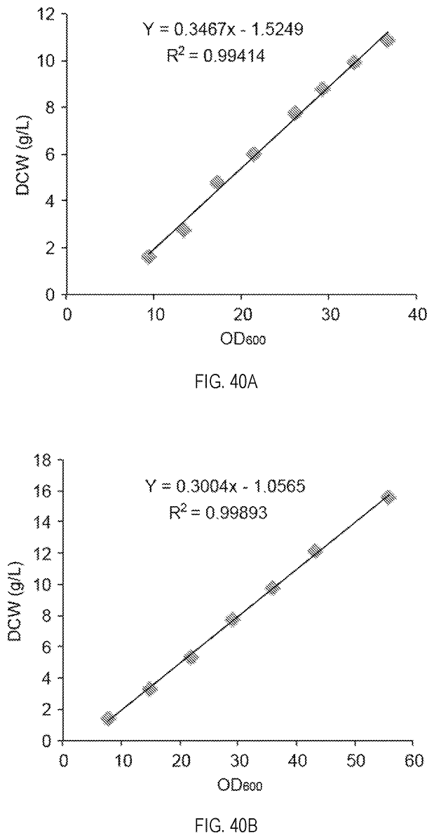

The production of carotenoids was expressed as grams per liter of fermentation broth (g/L) and milligrams per gram of dry cell weight (mg/g DCW). Optical densities were measured at 600 nm with Thermo Spectronic Genesys 20 (Thermo Scientific) and used to calculate cell mass (DCW=0.35×OD₆₀₀ for β-carotene and DCW=0.30×OD₆₀₀ for lycopene, FIGS. 40A-40B). The analysis and quantification of β-carotene was performed by HPLC (SHIMADZU LC-20 AT) equipped with a Kromasil C18 column (4.6 mm×250 mm) and UV/VIS detection at 450 nm. The mobile phase consisted of acetonitrile-methanol-isopropanol (5:3:2 v/v) with a flow rate of 1 mL/min at 40° C. The analysis and quantification of lycopene were performed with Spectramax M2e Microplate Reader (Molecular devices) or HPLC at 470 nm. The standard curves of β-carotene and lycopene (Sigma-Aldrich) were prepared by running the same extraction process as the samples.

### Quantitative Real-Time PCR

Real-time PCR (RT-PCR) was used to estimate the relative gene expression. mRNA extracted by MasterPure™ Yeast RNA purification kit (Lucigen, Wisconsin, USA) was used as the template. RT-PCR was carried out on an iCycler (Bio-Rad, USA) using iScript™ one-step RT-PCR kit with SYBR Green Supermix (Bio-Rad, USA) according to the manufacturer's instructions. ACT1 was used as an internal control gene for normalization. The relative gene expression was calculated using the comparative 2^{-ΔΔCT} or 2^{-ΔCT} method.

### In Vitro Enzymatic Assays

Yeast microsomes for in vitro enzymatic assays were prepared as described previously (Pompon et al., 1996). Briefly, strains harboring wild type or mutated CarRP were grown overnight in YNB media at 30° C. and then inoculated into 200 mL YNB media to an initial OD₆₀₀ of 0.1. After 24 h cultivation, cells were collected by centrifugation at 4,000 rpm for 10 min. Resuspension of the cells in TEK buffer (50 mM Tris-HCl, pH 6.8, 1 mM EDTA, 0.1 M KCl) followed, and the solution was kept at room temperature for 5 min. Afterwards, the cells were recovered by centrifugation, washed in TES buffer (50 mM Tris-HCl, pH 6.8, 1 mM EDTA, 0.6 M sorbitol), resuspended in TESM buffer (50 mM Tris-HCl, pH 6.8, 1 mM EDTA, 0.6 M sorbitol, 14 mM 2-mercaptoethanol), and left at room temperature for 10 min. Then, the cells were recovered once again by centrifugation, washed in extraction buffer (50 mM Tris-HCl, pH 6.8, 1 mM EDTA, 1 mM PMSF), and resuspended in extraction buffer. Glass beads were added to each sample, which were intermittently vortexed for 30 s and placed on ice for 30 s for a total of 15 repeats. The cell pellets were then discarded by centrifugation at 4,000 rpm, 4° C. for 10 min, and the supernatant was transferred to a 50 mL tube. The crude yeast microsomal fraction collected above was used for in vitro assays of lycopene cyclase. Standard enzyme assays were performed in a total volume of 200 μL containing 50 mM Tris-HCl (pH 6.8), 1 mM phenylmethylsulfonyl fluoride (PMSF) and 1 mg of microsomal proteins. Serial concentrations of lycopene (50˜350 mol/L) dissolved in dimethyl sulfoxide (DMSO) were used as the substrate. Reactions were initiated by substrate addition, incubated at 30° C. with gentle shaking for 16 h, and then terminated by adding 200 μL ethyl acetate. The solution was vortex for 10 min, and the organic phase was collected by centrifugation and analyzed by HPLC.

### Generation of Protein Variants

The variants were generated by analyzing the amino acid conserved and co-evolutionary information of this protein family from Position Specific Scoring Matrix (PSSM). Here the matrix was generated using psiblast from ncbi-blast-2.7.1+(Altschul et al., 1997) with uniref90 (Suzek et al., 2007) as the database and an E-value of 0.01 running 3 iterations. For all positions in the protein, the PSSM score which represents conservatism of amino acids was calculated for both lycopene cyclase and other homologous proteins of this family. The higher score indicates the more conservative of the amino acid in this position. The substitutions that could be replaced with more conserved amino acids based on the PSSM scores were screened. The different value between the potential substitutions and wild-type amino acids was calculated and the scores were sorted. All glycine substitutions were removed from the scoring. Top 25 scoring substitutions were combined into double substitutions randomly. A distance matrix was computed using the PAM30 substitution matrix and clustered using Agglomerative in sklearn (Pedregosa et al., 2011) into 25 clusters to minimize the number for test. The variants were chosen randomly within each cluster. The ranked 26-50 scoring substitutions were ordered as single mutational variants.

### Homology Model of Lycopene Cyclase

A homology model was generated of the lycopene cyclase using TrRosetta server (Yang et al., 2020) submitted the sequence of the R domain (1-239 amino acids) of CarRP.

### Calculation of the C/N Ratio in Media

The C/N ratio was calculated by referring to the composition ofYeast extract (Bacto™) and Peptone (Bacto™) in the BD Bionutrients™ technical manual (Table 10) (legzacy.bd.com/ds/technicalCenter/misc/lcn01558-bionutrients-manual.pdf). The carbon in Yeast extract and Peptone was ignored because of its extremely lower concentration relative to that of glucose. The total nitrogen in Yeast extract and Peptone is 10.9% and 15.4%, respectively. The C/N ratio was generated with the following formula. X, Y, and Z represent the concentration of glucose, yeast extract and peptone respectively.

$$\frac{Carbon of glucose}{Nitrogen of ( yeast extract + peptone )} = \frac{\frac{X}{180.156 g {mol}^{- 1}} \times 6}{\frac{( 10.9 \% \times Y + 15.4 \% \times Z )}{14 g {mol}^{- 1}}}$$

TABLE 6
List of heterologous enzymes used in Example 5.

| | Name used | Accession | |
|---|---|---|---|
| Enzyme | in this study | No. | Organism |
| Phytoene | CrtI | AY177424.1 | Xanthophyllomyces dendrorhous |
| dehydrogenase | CarB | AJ238028.1 | Mucor circinelloides |
| Phytoene synthase/ | CrtYB | AY177204.1 | Xanthophyllomyces dendrorhous |
| lycopene cyclase | CarRP | AJ250827.1 | Mucor circinelloides |
| GGPP synthase | GGPPpa | AAA21260.1 | Pantoea agglomerans |
| | GGPPtc | AF081514.1 | Taxus canadensis |
| | GGPPsa | D28748.1 | Sulfolobus acidocaldarius |
| | GGPPxd | DQ016502.1 | Xanthophyllomyces dendrorhous |
| | GGPPy1 | XM502923.1 | Yarrowia lipolytica |
| lycopene | EuCrtY | D90087.2 | Erwinia uredovora |
| beta-cyclase | PaCrtY | JX871358.1 | Pantoea agglomerans |
| | PfCrtY | CP002727.1 | Pseudomonas fulva |
| | HpCrtY | AY182008.1 | Haematococcus pluvialis |

TABLE 7
Strains and constructs used in Example 5.

| Strain name | Description of strains and plasmids | Source |
|---|---|---|
| polf | MatA, leu2-270, ura3-302, xpr2-322, axp-2 | Lab stock |
| polf-T | MatA, leu2-270, ura3-302, xpr2-322, axp-2, trpl | Example 5 |
| YLMA01 | polf-T, URA3::T_{Lip1}-CrtI-P_{GPD} + P_{TEF}-CrtYB-T_{XPR2} | Example 5 |
| YLMA02 | polf-T, URA3::T_{Lip1}-CarB-P_{GPD} + P_{TEF}-CarRP-T_{XPR2} | Example 5 |
| YLMA03 | YLMA02, LEU2::P_{TEF}-GGPPxd-T_{XPR2} | Example 5 |
| YLMA04 | YLMA03, TRP1::P_{TEF}-CarRP-T_{XPR2} | Example 5 |
| YLMA05 | YLMA03, TRP1::P_{TEF}-tCarRP-T_{XPR2} | Example 5 |
| YLMA06 | YLMA03, TRP1::P_{TEF}-mCarRP-T_{XPR2} | Example 5 |
| YLMA07 | YLMA03, TRP1::P_{TEF}-EuCrtY-T_{XPR2} | Example 5 |
| YLMA08 | YLMA03, TRP1::P_{TEF}-PaCrtY-T_{XPR2} | Example 5 |
| YLMA09 | YLMA03, TRP1::P_{TEF}-PfCrtY-T_{XPR2} | Example 5 |
| YLMA10 | YLMA03, TRP1::P_{TEF}-HpCrtY-T_{XPR2} | Example 5 |
| YLMA11 | polf-T, LEU2::P_{TEF}-GGPPxd-T_{XPR2} + T_{Lip1}-CarB-P_{GPD} + P_{TEF}- | Example 5 |
| | CarRP^{Y27R}-T_{XPR2} | |
| YLMA12 | polf-T, LEU2::P_{TEF}-GGPPxd-T_{XPR2} + T_{Lip1}-CarB-P_{GPD} + P_{TEF}- | Example 5 |
| | CarRP^{V175W}-T_{XPR2} | |
| YLMA13 | polf-T, LEU2::P_{TEF}-GGPPxd-T_{XPR2} + T_{Lip1}-CarB-P_{GPD} + P_{TEF}- | Example 5 |
| | CarRP^{T31R-F92W}-T_{XPR2} | |
| YLMA14 | YLMA11, URA3::T_{Lip1}-ERG12-P_{EXP1} + P_{TEFin}-tHMGR-T_{XPR2} + T_{Lip1}- | Example 5 |
| | ERG20-P_{EXP1} + P_{TEFin}-IDI-T_{XPR2} | |
| YLMA15 | YLMA14, TRP1::T_{Lip1}-CK-P_{EXP1} + P_{TEFin}-IPK-T_{XPR2} | Example 5 |
| YLMA16 | polf-T, TRPI::T_{Lip1}-CK-P_{EXP1} + P_{TEFin}-IPK-T_{XPR2} | Example 5 |
| YLMA17 | polf, LEU2::P_{TEF}-GGPPtc-T_{XPR2} | Example 5 |
| YLMA18 | polf, LEU2::P_{TEF}-GGPPpa-T_{XPR2} | Example 5 |
| YLMA19 | polf, LEU2::P_{TEF}-GGPPyl-T_{XPR2} | Example 5 |
| YLMA20 | polf, LEU2::P_{TEF}-GGPPsa-T_{XPR2} | Example 5 |
| YLMA21 | polf, LEU2::P_{TEF}-GGPPxd-T_{XPR2} | Example 5 |
| YLMA22 | YLMA02, LEU2::P_{TEF}-GGPPtc-T_{XPR2} | Example 5 |
| YLMA23 | YLMA02, LEU2::P_{TEF}-GGPPpa-T_{XPR2} | Example 5 |
| YLMA24 | YLMA02, LEU2::P_{TEF}-GGPPyl-T_{XPR2} | Example 5 |
| YLMA25 | YLMA02, LEU2::P_{TEF}-GGPPsa-T_{XPR2} | Example 5 |
| YLMA26 | polf-T, LEU2::P_{TEF}-GGPPsa-T_{XPR2} + T_{Lip1}-CarB-P_{GPD} + P_{TEF}- | Example 5 |
| | CarRP-T_{XPR2} | |
| YLMA27 | YLMA26, TRP1::P_{TEFin}-GGPPsa-T_{XPR2} + T_{Lip1}-CarB-P_{EXP1} + P_{TEFin}- | Example 5 |
| | CarRP-T_{XPR2} | |
| YLMA28 | YLMA27, URA3::P_{TEFin}-GGPPsa-T_{XPR2} + T_{Lip1}-CarB-P_{EXP1} + P_{TEFin}- | Example 5 |
| | CarRP-T_{XPR2} | |
| YLMA29 | YLMA28, ura⁻/leu⁻/trp⁻ | Example 5 |
| YLMA30 | YLMA29, URA3::T_{Lip1}-ERG12-P_{EXP1} + P_{TEFin}-tHMGR-T_{XPR2} + T_{Lip1}- | Example 5 |
| | ERG20-P_{EXP1} + P_{TEFin}-IDI-T_{XPR2} | |
| YLMA31 | YLMA30, TRP1::T_{Lip1}-CK-P_{EXP1} + P_{TEFin}-IPK-T_{XPR2} | Example 5 |
| YLMA32 | polf-T, LEU2::P_{TEF}-GGPPxd-T_{XPR2} + T_{Lip1}-CarB-P_{GPD} + P_{TEF}- | Example 5 |
| | CarRP^{E78K}-T_{XPR2} | |
| YLMA33 | YLMA32, URA3::T_{Lip1}-ERG12-P_{EXP1} + P_{TEFin}-tHMGR-T_{XPR2} + T_{Lip1}- | Example 5 |
| | ERG20-P_{EXP1} + P_{TEFin}-IDI-T_{XPR2} | |
| YLMA34 | YLMA33, TRP1::T_{Lip1}-CK-P_{EXP1} + P_{TEFin}-IPK-T_{XPR2} | Example 5 |

TABLE 8
Summary of carotenoid production in microorganisms.

| | | Fermentation | Titer | Content | Productivity | |
|---|---|---|---|---|---|---|
| Carotenoids | Host | scale | (g/L) | (mg/g DCW) | (g/L/h)^{a} | References |
| B-carotene | Y. | 3 L fermenter | 39.5 | 494 | 0.165 | Example 5 |
| | lipolytica | 5 L fermenter | 6.5 | 90 | 0.045 | Li et al. |
| | | | | | | 2016 |
| | | 2 L fermenter | 4 | 50 | 0.014 | Liu et al., |
| | | | | | | 2019 |
| | S. | 5 L fermenter | 1.2 | 21 | 0.01 | Wu et al. |
| | cerevisiae | | | | | 2016 |
| | E. coli | 5 L fermenter | 3.2 | — | 0.04 | Keasling et |
| | | | | | | al., 2010 |
| | | 7 L fermenter | 2.1 | 60 | 0.021 | Reed et al., |
| | | | | | | 2010 |
| | | 5 L fermenter | 2 | — | 0.015 | Boutin et |
| | | | | | | al., 2005 |
| Lycopene | Y. | 3 L fermenter | 17.6 | 313 | 0.073 | Example 5 |
| | lipolytica | 3 L fermenter | 4.2 | — | 0.016 | Belzer et |
| | | | | | | al., 2013 |
| | S. | 7 L fermenter | 3.28 | — | 0.02 | Singh et |
| | cerevisiae | | | | | al., 2013 |
| | | 7 L fermenter | 2.37 | 73.3 | 0.018 | Mateo et |
| | | | | | | al., 2004 |
| | | 7 L fermenter | 2.3 | — | 0.015 | Dougulis et |
| | | | | | | al., 2011 |
| | | 5 L fermenter | 1.65 | 55.6 | 0.014 | Gao et al., |
| | | | | | | 2009 |
| | | 5 L fermenter | 1.61 | 24.4 | 0.013 | Nielsen et |
| | | | | | | al., 2010 |
| | E. coli | 7 L fermenter | 3.52 | 50.6 | 0.029 | Kim et al., |
| | | | | | | 2007 |

ªProductivity is calculated as the total carotenoid produced divided by total fermentation time.

TABLE 9
Primers used in Example 5.

| | |
|---|---|
| Crispr-trp-F | GGGTCGGCGCAGGTTGACGTACGCCGAGGAGTGGTACCGGGTTT |
| | TAGAGCTAGAAATAGC (SEQ ID NO: 31) |
| Crispr-trp-R | GCTATTTCTAGCTCTAAAACCCGGTACCACTCCTCGGCGTACGTCA |
| | ACCTGCGCCGACCC (SEQ ID NO: 32) |
| CrtI-F | ATGAACCAGATGCATAGCACTTAGAAGGCCAGGATGCCCA |
| | (SEQ ID NO: 33) |
| CrtI-R | CATCAACAGTATCTACACGCATGGGCAAGGAACAGGACCA |
| | (SEQ ID NO: 34) |
| CrtYB-F | CATTCAAAGGATCCCCTAGGATGACCGCTCTGGCCTACTA |
| | (SEQ ID NO: 35) |
| CrtYB-R | GCAAGACCGGCAACGTGGGGTTACTGGCCCTCCCAGCCAG |
| | (SEQ ID NO: 36) |
| CarB-F | ATGAACCAGATGCATAGCACTTAGATCACGTTAGAGTTGT |
| | (SEQ ID NO: 37) |
| CarB-R | CATCAACAGTATCTACACGCATGTCCAAGAAGCACATCGT |
| | (SEQ ID NO: 38) |
| CarRP-F | CATTCAAAGGATCCCCTAGGATGCTGCTGACCTACATGGA |
| | (SEQ ID NO: 39) |
| CarRP-R | GCAAGACCGGCAACGTGGGGTTAGATGGTGTTCAGGTTTC |
| | (SEQ ID NO: 40) |
| GGPPxd-F | CATTCAAAGGATCCCCTAGGATGGACTACGCCAACATCCT |
| | (SEQ ID NO: 41) |
| GGPPxd-R | GCAAGACCGGCAACGTGGGGTTACAGGGGAATGTCGGCCA |
| | (SEQ ID NO: 42) |
| tCarRP-F | CATTCAAAGGATCCCCTAGGATGCTGCTGACCTACATGGA |
| | (SEQ ID NO: 39) |
| tCarRP-R | GCAAGACCGGCAACGTGGGGTTACAGCTCTTGTCGCACGTCG |
| | (SEQ ID NO: 43) |
| mCarRP-1-F | CATTCAAAGGATCCCCTAGGATGCTGCTGACCTACATGGA |
| | (SEQ ID NO: 39) |
| mCarRP-1-R | TCGCTCGAGGCCCCACTTGTAGCCGTCCAG (SEQ ID NO: 44) |
| mCarRP-2-F | ACAAGTGGGGCCTCGAGCGACGATCTATCC (SEQ ID NO: 45) |
| mCarRP-2-R | GCAAGACCGGCAACGTGGGGTTAGATGGTGTTCAGGTTTC |
| | (SEQ ID NO: 40) |
| EuCrtY-F | CATTCAAAGGATCCCCTAGGATGCAGCCCCACTACGACCT |
| | (SEQ ID NO: 46) |
| EuCrtY-R | GCAAGACCGGCAACGTGGGGTTATCGGTGAGTGGTCATGA |
| | (SEQ ID NO: 47) |
| PaCrtY-F | CATTCAAAGGATCCCCTAGGATGCCCCGATACGACCTGAT |
| | (SEQ ID NO: 48) |
| PaCrtY-R | GCAAGACCGGCAACGTGGGGTTACTGCATGGCCTGCTGTC |
| | (SEQ ID NO: 49) |
| PfCrtY-F | CATTCAAAGGATCCCCTAGGATGTCTTACGACCTGATCCT |
| | (SEQ ID NO: 50) |
| PfCrt Y-R | GCAAGACCGGCAACGTGGGGTTAAGATCGCATCTCGAAGT |
| | (SEQ ID NO: 51) |
| HpCrtY-F | CATTCAAAGGATCCCCTAGGATGCTGTCTCCCCTGCAGCG |
| | (SEQ ID NO: 52) |
| HpCrtY-R | GCAAGACCGGCAACGTGGGGTTACTTGATCATGGCTGGAG |
| | (SEQ ID NO: 53) |
| GGPPtc-F | CATTCAAAGGATCCCCTAGGATGGCCTACACCGCCATGGC |
| | (SEQ ID NO: 54) |
| GGPPtc-R | GCAAGACCGGCAACGTGGGGTTAGTTCTGTCGGAAGGCAA |
| | (SEQ ID NO: 55) |
| GGPPpa-F | CATTCAAAGGATCCCCTAGGATGGTCTCTGGATCTAAGGC |
| | (SEQ ID NO: 56) |
| GGPPpa-R | GCAAGACCGGCAACGTGGGGTCAGGCAATCTTCATGACAG |
| | (SEQ ID NO: 57) |
| GGPPyl-F | CATTCAAAGGATCCCCTAGGATGGATTATAACAGCGCGGA |
| | (SEQ ID NO: 58) |
| GGPPy1-R | GCAAGACCGGCAACGTGGGGTCACTGCGCATCCTCAAAGT |
| | (SEQ ID NO: 59) |
| GGPPsa-F | CATTCAAAGGATCCCCTAGGATGTCCTACTTCGACAACTA |
| | (SEQ ID NO: 60) |
| GGPPsa-R | GCAAGACCGGCAACGTGGGGTTACTTTCGTCGTCGAATGG |
| | (SEQ ID NO: 61) |
| ERG12-F | ATGAACCAGATGCATAGCACCTAATGGGTCCAGGGACCGA |
| | (SEQ ID NO: 62) |
| ERG12-R | ACACAAGACATATCTACAGCATGGACTACATCATTTCGGC |
| | (SEQ ID NO: 63) |
| tHMGR-F | TTTTGCAGTACTAACCGCAGACCCAGTCTGTGAAGGTGGT |
| | (SEQ ID NO: 64) |
| tHMGR-R | GCAAGACCGGCAACGTGGGGCTATGACCGTATGCAAATAT |
| | (SEQ ID NO: 65) |
| ERG20-F | ATGAACCAGATGCATAGCACCTACTTCTGTCGCTTGTAAA |
| | (SEQ ID NO: 66) |
| ERG20-R | ACACAAGACATATCTACAGCATGTCCAAGGCGAAATTCGA |
| | (SEQ ID NO: 67) |
| IDI-F | TTTTGCAGTACTAACCGCAGACGACGTCTTACAGCGACAA |
| | (SEQ ID NO: 68) |
| IDI-R | GCAAGACCGGCAACGTGGGGCTACTTGATCCACCGCCGAA |
| | (SEQ ID NO: 69) |
| CK-F | ATGAACCAGATGCATAGCACCTACGCATTAATAAACCGGG |
| | (SEQ ID NO: 70) |
| CK-R | ACACAAGACATATCTACAGCATGGCCCCTCAACCCGACCA |
| | (SEQ ID NO: 71) |
| IPK-F | TTTTGCAGTACTAACCGCAGGAACTGAACATCTCTGAGTC |
| | (SEQ ID NO: 72) |
| IPK-R | GCAAGACCGGCAACGTGGGGTTACTTGGAGAATCGGATGA |
| | (SEQ ID NO: 73) |
| ACT-RT-F | GAGTCACCGGTATCGTTC |
| | (SEQ ID NO: 74) |
| ACT-RT-R | GCGGAGTTGGTGAAAGAG |
| | (SEQ ID NO: 75) |
| CarRP-RT-F | CTGGCCCTGCTGTGGTTCGG |
| | (SEQ ID NO: 76) |
| CarRP-RT-R | CAGGCAGATGGGGCACCACG |
| | (SEQ ID NO: 77) |

TABLE 10
Composition of Yeast extract (Bacto ™) and Peptone (Bacto ™).

| | Yeast | | | Yeast | |
|---|---|---|---|---|---|
| Composition | extract | Peptone | Composition | extract | Peptone |
| Total Nitrogen (%) | 10.9 | 15.4 | Glutamine (% Free) | 0.2 | 0.0 |
| Amino Nitrogen (%) | 6.0 | 3.5 | Glycine (% Free) | 1.0 | 0.7 |
| AN/TN | 0.55 | 0.20 | Glycine (% Total) | 3.0 | 15.9 |
| Total Carbohydrate (mg/g) | 163.3 | 6.29 | Histidine (% Free) | 0.4 | 0.2 |
| Ash (%) | 11.2 | 3.8 | Histidine (% Total) | 1.3 | 0.8 |
| Loss on Drying (%) | 3.1 | 2.7 | Isoleucine (% Free) | 1.8 | 0.6 |
| NaCl (%) | 0.1 | 1.7 | Isoleucine (% Total) | 3.0 | 2.1 |
| pH (1% Solution) | 6.7 | 7.1 | Leucine (% Free) | 3.0 | 1.6 |
| Calcium (μg/g) | 130 | 30 | Leucine (% Total) | 4.1 | 3.8 |
| Iron (μg/g) | 55.3 | 7.8 | Lysine (% Free) | 1.9 | 2.2 |
| Magnesium (μg/g) | 750 | 17 | Lysine (% Total) | 4.6 | 3.4 |
| Potassium (μg/g) | 31950 | 2487 | Methionine (% Free) | 0.6 | 0.3 |
| Sodium (μg/g) | 4900 | 18127 | Methionine (% Total) | 0.8 | 0.7 |
| Chloride (%) | 0.38 | 0.90 | Phenylalanine (% Free) | 2.0 | 1.4 |
| Sulfate (%) | 0.09 | 0.32 | Phenylalanine (% Total) | 2.6 | 2.8 |
| Phosphate (%) | 3.27 | 0.40 | Proline (% Free) | 0.8 | 0.3 |
| Alanine (% Free) | 4.4 | 1.2 | Proline (% Total) | 2.0 | 8.8 |
| Alanine (% Total) | 5.6 | 9.2 | Serine (% Free) | 1.3 | 0.4 |
| Arginine (% Free) | 1.4 | 2.8 | Serine (% Total) | 1.6 | 1.5 |
| Arginine (% Total) | 2.6 | 5.8 | Threonine (% Free) | 1.1 | 0.3 |
| Asparagine (% Free) | 1.0 | 0.3 | Threonine (% Total) | 1.6 | 1.1 |
| Aspartic Acid (% Free) | 1.6 | 0.3 | Tryptophan (% Free) | 0.5 | 0.3 |
| Aspartic Acid (% Total) | 5.3 | 5.0 | Tyrosine (% Free) | 0.8 | 0.5 |
| Cystine (% Free) | 0.2 | 0.0 | Tyrosine (% Total) | 1.2 | 0.6 |
| Glutamic Acid (% Free) | 6.6 | 0.7 | Valine (% Free) | 2.2 | 0.7 |
| Glutamic Acid (% Total) | 9.4 | 8.1 | Valine (% Total) | 3.5 | 2.8 |

### REFERENCES

1. Menchik, P.; Zuber, T.; Zuber, A.; Moraru, C. I. Short Communication: Composition of Coproduct Streams from Dairy Processing: Acid Whey and Milk Permeate. J. Dairy Sci. 2019, 102 (5), 3978-3984. doi.org/10.3168/jds.2018-15951.
2. Lievore, P.; Sim6es, D. R. S.; Silva, K. M.; Drunkler, N. L.; Barana, A. C.; Nogueira, A.; Demiate, I. M. Chemical Characterisation and Application of Acid Whey in Fermented Milk. J. Food Sci. Technol. 2015, 52 (4), 2083-2092. doi.org/10.1007/s13197-013-1244-z.
3. Lindsay, M. J.; Walker, T. W.; Dumesic, J. A.; Rankin, S. A.; Huber, G. W. Production of Monosaccharides and Whey Protein from Acid Whey Waste Streams in the Dairy Industry. Green Chem. 2018, 20 (8), 1824-1834. doi.org/10.1039/c8gc00517f.
4. Xu, J.; Hao, J.; Guzman, J. J. L.; Spirito, C. M.; Harroff, L. A.; Angenent, L. T. Temperature-Phased Conversion of Acid Whey Waste Into Medium-Chain Carboxylic Acids via Lactic Acid: No External e-Donor. Joule 2018, 2 (2), 280-295. doi.org/10.1016/j.joule.2017.11.008.
5. Bentahar, J.; Doyen, A.; Beaulieu, L.; Deschenes, J. S. Acid Whey Permeate: An Alternative Growth Medium for Microalgae Tetradesmus Obliquus and Production of β-Galactosidase. Algal Res. 2019, 41, 101559. doi.org/10.1016/j.algal.2019.101559.
6. Fischer, C.; Kleinschmidt, T. Synthesis of Galactooligosaccharides Using Sweet and Acid Whey as a Substrate. Int. Dairy J. 2015, 48, 15-22. doi.org/10.1016/j.idairyj.2015.01.003.
7. Skryplonek, K.; Dmytrów, I.; Mituniewicz-Małek, A. Probiotic Fermented Beverages Based on Acid Whey. J. Dairy Sci. 2019, 102 (9), 7773-7780. doi.org/10.3168/jds.2019-16385.
8. Carotenoids Market|2020-2027| Industry Report Covid Insights mordorintelligence.com/industry-reports/carotenoids-market-industry (accessed Feb. 18, 2021).
9. Mano, J.; Liu, N.; Hammond, J. H.; Currie, D. H.; Stephanopoulos, G. Engineering Yarrowia Lipolytica for the Utilization of Acid Whey. Metab. Eng. 2020, 57, 43-50. doi.org/10.1016/j.ymben.2019.09.010.
10. Larroude, M.; Celinska, E.; Back, A.; Thomas, S.; Nicaud, J. M.; Ledesma-Amaro, R. A Synthetic Biology Approach to Transform Yarrowia Lipolytica into a Competitive Biotechnological Producer of β-Carotene. Biotechnol. Bioeng. 2018, 115 (2), 464-472. doi.org/10.1002/bit.26473.
11. Yamada, E. A.; Sgarbieri, V. C. Yeast (Saccharomyces Cerevisiae) Protein Concentrate: Preparation, Chemical Composition, and Nutritional and Functional Properties. J. Agric. Food Chem. 2005, 53 (10), 3931-3936. doi.org/10.1021/jf0400821.
12. Gongalves, F. A. G.; Colen, G.; Takahashi, J. A. Yarrowia Lipolytica and Its Multiple Applications in the Biotechnological Industry. Sci. World J. 2014, 2014. doi.org/10.1155/2014/476207.
13. Qiao, K.; Abidi, S. H. I.; Liu, H.; Zhang, H.; Chakraborty, S.; Watson, N.; Ajikumar, P. K.; Stephanopoulos, G. Engineering Lipid Overproduction in the Oleaginous Yeast Yarrowia Lipolytica. Metab. Eng. 2015, 29, 56-65. doi.org/10.1016/j.ymben.2015.02.005.
14. Tai, M.; Stephanopoulos, G. Engineering the Push and Pull of Lipid Biosynthesis in Oleaginous Yeast Yarrowia Lipolytica for Biofuel Production. Metab. Eng. 2013, 15 (1), 1-9. doi.org/10.1016/j.ymben.2012.08.007.
15. U.S. Greek Yogurt Market—Statistics & Facts Statista. statista.com/topics/2351/greek-yogurt/.
16. Menchik, P., Zuber, T., Zuber, A. & Moraru, C. I. Short communication: Composition of coproduct streams from dairy processing: Acid whey and milk permeate. J. Dairy Sci. 102,3978-3984(2019).
17. Lievore, P. et al. Chemical characterisation and application of acid whey in fermented milk. J. Food Sci. Technol. 52, 2083-2092 (2015).
18. Maoka, T. Carotenoids as natural functional pigments. Journal of Natural Medicines vol. 741-16 (2020).
19. Bogacz-Radomska, L. & Harasym, J. β-Carotene—properties and production methods. Food Qual. Saf. 2, 69-74 (2018).
20. Li, C., Swofford, C. A. & Sinskey, A. J. Modular engineering for microbial production of carotenoids. Metabolic Engineering Communications vol. 10 e00118 (2020).
21. Mano, J., Liu, N., Hammond, J. H., Currie, D. H. & Stephanopoulos, G. Engineering Yarrowia lipolytica for the utilization of acid whey. Metab. Eng. 57, 43-50 (2020).
22. Anselmo, A. C. et al. A heat-stable microparticle platform for oral micronutrient delivery. Sci. Transl. Med. 11, (2019).
23. Tai, M. & Stephanopoulos, G. Engineering the push and pull of lipid biosynthesis in oleaginous yeast Yarrowia lipolytica for biofuel production. Metab. Eng. 15, 1-9 (2013).
24. Xu, P., Qiao, K., Ahn, W. S. & Stephanopoulos, G. Engineering Yarrowia lipolytica as a platform for synthesis of drop-in transportation fuels and oleochemicals. Proc. Natl. Acad. Sci. U.S.A 113, 10848-10853 (2016).
25. Qiao, K., Wasylenko, T. M., Zhou, K., Xu, P. & Stephanopoulos, G. Lipid production in Yarrowia lipolytica is maximized by engineering cytosolic redox metabolism. Nat. Biotechnol. 35, 173-177 (2017).
26. Qiao, K. et al. Engineering lipid overproduction in the oleaginous yeast Yarrowia lipolytica. Metab. Eng. 29, 56-65 (2015).
27. Luo, Z. et al. Enhancing isoprenoid synthesis in Yarrowia lipolytica by expressing the isopentenol utilization pathway and modulating intracellular hydrophobicity. Metab. Eng. 61, 344-351 (2020).
28. Larroude, M. et al. A synthetic biology approach to transform Yarrowia lipolytica into a competitive biotechnological producer of β-carotene. Biotechnol. Bioeng. 115, 464-472 (2018).
29. Groenewald, M. et al. Yarrowia lipolytica: Safety assessment of an oleaginous yeast with a great industrial potential. Critical Reviews in Microbiology vol. 40 187-206 (2014)
30. Carotenoids Market|2020-2027|Industry Report Covid Insights. mordorintelligence.com/industry-reports/carotenoids-market-industry.
31. Carotenoids Market Size, Revenue|Global Industry Trends [2020-2027]. fortunebusinessinsights.com/industry-reports/carotenoids-market-100180.
32. Groenewald, M. et al. Yarrowia lipolytica: Safety assessment of an oleaginous yeast with a great industrial potential. Critical Reviews in Microbiology vol. 40 187-206 (2014).
33. Xue, Z. et al. Production of omega-3 eicosapentaenoic acid by metabolic engineering of Yarrowia lipolytica. Nat. Biotechnol. 31, 734-740 (2013).
34. Xie, D., Jackson, E. N. & Zhu, Q. Sustainable source of omega-3 eicosapentaenoic acid from metabolically engineered Yarrowia lipolytica: from fundamental research to commercial production. Appl. Microbiol. Biotechnol. 99, 1599-1610 (2015).
35. Ritala, A., Häkkinen, S. T., Toivari, M. & Wiebe, M. G. Single Cell Protein—State-of-the-Art, Industrial Landscape and Patents 2001-2016. Front. Microbiol. 8, 2009 (2017).
36. Lindsay, M. J., Walker, T. W., Dumesic, J. A., Rankin, S. A. & Huber, G. W. Production of monosaccharides and whey protein from acid whey waste streams in the dairy industry. Green Chem. 20, 1824-1834 (2018).
37. Xu, J. et al. Temperature-Phased Conversion of Acid Whey Waste Into Medium-Chain Carboxylic Acids via Lactic Acid: No External e-Donor. Joule 2, 280-295 (2018).
38. Bentahar, J., Doyen, A., Beaulieu, L. & Deschênes, J. S. Acid whey permeate: An alternative growth medium for microalgae Tetradesmus obliquus and production of β-galactosidase. Algal Res. 41, 101559 (2019).
39. Fischer, C. & Kleinschmidt, T. Synthesis of galactooligosaccharides using sweet and acid whey as a substrate. Int. Dairy J. 48, 15-22 (2015).
40. Skryplonek, K., Dmytrów, I. & Mituniewicz-Małek, A. Probiotic fermented beverages based on acid whey. J. Dairy Sci. 102, 7773-7780 (2019).
41. Xu, P., Qiao, K. & Stephanopoulos, G. Engineering oxidative stress defense pathways to build a robust lipid production platform in Yarrowia lipolytica. Biotechnol. Bioeng. 114, 1521-1530 (2017).
42. Heinzle, E. et al. Ecological and economic objective functions for screening in integrated development of fine chemical processes. 1. Flexible and expandable framework using indices. Ind. Eng. Chem. Res. 37, 3395-3407 (1998).
43. Abdel-Mawgoud, A. M., Markham, K. A., Palmer, C. M., Liu, N., Stephanopoulos, G., Alper, H. S., 2018. Metabolic engineering in the host Yarrowia lipolytica. Metab. Eng. 50, 192-208.
44. Ajikumar, P. K., Xiao, W.-H., Tyo, K. E., Wang, Y., Simeon, F., Leonard, E., Mucha, O., Phon, T. H., Pfeifer, B., Stephanopoulos, G., 2010. Isoprenoid pathway optimization for Taxol precursor overproduction in Escherichia coli. Science. 330, 70-74.
45. Ambati, R. R., Phang, S.-M., Ravi, S., Aswathanarayana, R. G., 2014. Astaxanthin: sources, extraction, stability, biological activities and its commercial applications—a review. Mar. Drugs. 12, 128-152.
46. Asker, D., 2017. Isolation and characterization of a novel, highly selective astaxanthin-producing marine bacterium. J. Agric. Food. Chem. 65, 9101-9109.
47. Atanasov, A. G., Waltenberger, B., Pferschy-Wenzig, E.-M., Linder, T., Wawrosch, C., Uhrin, P., Temml, V., Wang, L., Schwaiger, S., Heiss, E. H., 2015. Discovery and resupply of pharmacologically active plant-derived natural products: A review. Biotechnol. Adv. 33, 1582-1614.
48. Avalos, J. L., Fink, G. R., Stephanopoulos, G., 2013. Compartmentalization of metabolic pathways in yeast mitochondria improves the production of branched-chain alcohols. Nat. Biotechnol. 31, 335.
49. Ayer, A., Sanwald, J., Pillay, B. A., Meyer, A. J., Perrone, G. G., Dawes, I. W., 2013.
Distinct redox regulation in sub-cellular compartments in response to various stress conditions in Saccharomyces cerevisiae. PLoS One. 8.
50. Bayer, T. S., Widmaier, D. M., Temme, K., Mirsky, E. A., Santi, D. V., Voigt, C. A., 2009. Synthesis of methyl halides from biomass using engineered microbes. J. Am. Chem. Soc. 131, 6508-6515.
51. Bennedsen, M., Wang, X., Willén, R., Wadström, T., Andersen, L. P., 2000. Treatment of H. pylori infected mice with antioxidant astaxanthin reduces gastric inflammation, bacterial load and modulates cytokine release by splenocytes. Immunol. Lett. 70, 185-189.
52. Cao, X., Yang, S., Cao, C., Zhou, Y. J., 2020. Harnessing sub-organelle metabolism for biosynthesis of isoprenoids in yeast. Synth. Syst. Biotechnol. 5, 179-186.
53. Chang, J.-J., Thia, C., Lin, H.-Y., Liu, H.-L., Ho, F.-J., Wu, J.-T., Shih, M.-C., Li, W.-H., Huang, C.-C., 2015. Integrating an algal β-carotene hydroxylase gene into a designed carotenoid-biosynthesis pathway increases carotenoid production in yeast. Bioresour. Technol. 184, 2-8.
54. Chemler, J. A., Koffas, M. A., 2008. Metabolic engineering for plant natural product biosynthesis in microbes. Curr. Opin. Biotechnol. 19, 597-605.
55. Chew, B., Park, J., Wong, M., Wong, T., 1999. A comparison of the anticancer activities of dietary beta-carotene, canthaxanthin and astaxanthin in mice in vivo. Anticancer Res. 19, 1849-1853.
56. Choi, S.-k., Nishida, Y., Matsuda, S., Adachi, K., Kasai, H., Peng, X., Komemushi, S., Miki, W., Misawa, N., 2005. Characterization of β-Carotene Ketolases, CrtW, from Marine Bacteria by Complementation Analysis in Escherichiacoli. Mar. Biotechnol. 7, 515-522.
57. Cragg, G. M., 1998. Paclitaxel (Taxol®): a success story with valuable lessons for natural product drug discovery and development. Med. Res. Rev. 18, 315-331.
58. Dhingra, V., Rao, K. V., Narasu, M. L., 1999. Current status of artemisinin and its derivatives as antimalarial drugs. Life Sci. 66, 279-300.
59. Diao, J., Song, X., Zhang, L., Cui, J., Chen, L., Zhang, W., 2020. Tailoring cyanobacteria as a new platform for highly efficient synthesis of astaxanthin. Metab. Eng.
60. Dzubak, P., Hajduch, M., Vydra, D., Hustova, A., Kvasnica, M., Biedermann, D., Markova, L., Urban, M., Sarek, J., 2006. Pharmacological activities of natural triterpenoids and their therapeutic implications. Nat. Prod. Rep. 23, 394-411.
61. Farhi, M., Marhevka, E., Masci, T., Marcos, E., Eyal, Y., Ovadis, M., Abeliovich, H., Vainstein, A., 2011. Harnessing yeast subcellular compartments for the production of plant terpenoids. Metab. Eng. 13, 474-481.
62. Gong, Z., Wang, H., Tang, J., Bi, C., Li, Q., Zhang, X., 2020. Coordinated expression of astaxanthin biosynthesis genes for improved astaxanthin production in Escherichia coli. J. Agric. Food. Chem. 68, 14917-14927.
63. Hama, S., Takahashi, K., Inai, Y., Shiota, K., Sakamoto, R., Yamada, A., Tsuchiya, H., Kanamura, K., Yamashita, E., Kogure, K., 2012. Protective effects of topical application of a poorly soluble antioxidant astaxanthin liposomal formulation on ultraviolet-induced skin damage. J. Pharm. Sci. 101, 2909-2916.
64. Hammer, S. K., Avalos, J. L., 2017. Harnessing yeast organelles for metabolic engineering. Nat. Chem. Biol. 13, 823.
65. Henke, N. A., Wiebe, D., Pdrez-Garcia, F., Peters-Wendisch, P., Wendisch, V. F., 2018. Coproduction of cell-bound and secreted value-added compounds: Simultaneous production of carotenoids and amino acids by Corynebacterium glutamicum. Bioresour. Technol. 247, 744-752.
66. Jiang, G., Yang, Z., Wang, Y., Yao, M., Chen, Y., Xiao, W., Yuan, Y., 2020. Enhanced astaxanthin production in yeast via combined mutagenesis and evolution. Biochem. Eng. J. 156, 107519.
67. Jin, J., Wang, Y., Yao, M., Gu, X., Li, B., Liu, H., Ding, M., Xiao, W., Yuan, Y., 2018. Astaxanthin overproduction in yeast by strain engineering and new gene target uncovering. Biotechnol. Biofuels. 11, 230.
68. Kildegaard, K. R., Adiego-Pérez, B., Belda, D. D., Khangura, J. K., Holkenbrink, C., Borodina, I., 2017. Engineering of Yarrowia lipolytica for production of astaxanthin. Synth. Syst. Biotechnol. 2, 287-294.
69. Larroude, M., Celinska, E., Back, A., Thomas, S., Nicaud, J. M., Ledesma—Amaro, R., 2018. A synthetic biology approach to transform Yarrowia lipolytica into a competitive biotechnological producer of β-carotene. Biotechnol. Bioeng. 115, 464-472.
70. Lemuth, K., Steuer, K., Albermann, C., 2011. Engineering of a plasmid-free Escherichia coli strain for improved in vivo biosynthesis of astaxanthin. Microb. Cell Fact. 10, 29.
71. Li, N., Han, Z., O'Donnell, T. J., Kurasaki, R., Kajihara, L., Williams, P. G., Tang, Y., Su, W. W., 2020. Production and excretion of astaxanthin by engineered Y arrowia lipolytica using plant oil as both the carbon source and the biocompatible extractant. Appl. Microbiol. Biotechnol., 1-13.
72. Liu, G.-S., Li, T., Zhou, W., Jiang, M., Tao, X.-Y., Liu, M., Zhao, M., Ren, Y.-H., Gao, B., Wang, F.-Q., 2020. The yeast peroxisome: A dynamic storage depot and subcellular factory for squalene overproduction. Metab. Eng. 57, 151-161.
73. Lu, Q., Bu, Y.-F., Liu, J.-Z., 2017. Metabolic engineering of Escherichia coli for producing astaxanthin as the predominant carotenoid. Mar. Drugs. 15, 296.
74. Ma, T., Zhou, Y., Li, X., Zhu, F., Cheng, Y., Liu, Y., Deng, Z., Liu, T., 2016. Genome mining of astaxanthin biosynthetic genes from Sphingomonas sp. ATCC 55669 for heterologous overproduction in Escherichia coli. Biotechnology journal. 11, 228-237.
75. Martin, V. J., Pitera, D. J., Withers, S. T., Newman, J. D., Keasling, J. D., 2003. Engineering a mevalonate pathway in Escherichia coli for production of terpenoids. Nat. Biotechnol. 21, 796-802.
76. Nogueira, M., Enfissi, E. M. A., Welsch, R., Beyer, P., Zurbriggen, M. D., Fraser, P. D., 2019. Construction of a fusion enzyme for astaxanthin formation and its characterisation in microbial and plant hosts: A new tool for engineering ketocarotenoids. Metab. Eng. 52, 243-252.
77. Park, S. Y., Binkley, R. M., Kim, W. J., Lee, M. H., Lee, S. Y., 2018. Metabolic engineering of Escherichia coli for high-level astaxanthin production with high productivity. Metab. Eng. 49, 105-115.
78. Qi, D.-D., Jin, J., Liu, D., Jia, B., Yuan, Y.-J., 2020. In vitro and in vivo recombination of heterologous modules for improving biosynthesis of astaxanthin in yeast. Microb. Cell Fact. 19, 1-12.
79. Scaife, M. A., Burja, A. M., Wright, P. C., 2009. Characterization of cyanobacterial (3-carotene ketolase and hydroxylase genes in Escherichia coli, and their application for astaxanthin biosynthesis. Biotechnol. Bioeng. 103, 944-955.
80. Scaife, M. A., Ma, C. A., Ninlayarn, T., Wright, P. C., Armenta, R. E., 2012. Comparative analysis of β-carotene hydroxylase genes for astaxanthin biosynthesis. J. Nat. Prod. 75, 1117-1124.
81. Sheng, J., Stevens, J., Feng, X., 2016. Pathway compartmentalization in peroxisome of Saccharomyces cerevisiae to produce versatile medium chain fatty alcohols. Sci. Rep. 6, 1-11.
82. Szczebara, F. M., Chandelier, C., Villeret, C., Masurel, A., Bourot, S., Duport, C., Blanchard, S., Groisillier, A., Testet, E., Costaglioli, P., 2003. Total biosynthesis of hydrocortisone from a simple carbon source in yeast. Nat. Biotechnol. 21, 143-149.
83. Thodey, K., Galanie, S., Smolke, C. D., 2014. A microbial biomanufacturing platform for natural and semisynthetic opioids. Nat. Chem. Biol. 10, 837.
84. Tramontin, L. R. R., Kildegaard, K. R., Sudarsan, S., Borodina, I., 2019. Enhancement of astaxanthin biosynthesis in oleaginous yeast Yarrowia lipolytica via microalgal pathway. Microorganisms. 7, 472.
85. Ukibe, K., Hashida, K., Yoshida, N., Takagi, H., 2009. Metabolic engineering of Saccharomyces cerevisiae for astaxanthin production and oxidative stress tolerance. Appl. Environ. Microbiol. 75, 7205-7211.
86. Wang, R., Gu, X., Yao, M., Pan, C., Liu, H., Xiao, W., Wang, Y., Yuan, Y., 2017. Engineering of β-carotene hydroxylase and ketolase for astaxanthin overproduction in Saccharomyces cerevisiae. Front. Chem. Sci. Eng. 11, 89-99.
87. Xu, P., Qiao, K., Ahn, W. S., Stephanopoulos, G., 2016. Engineering Yarrowia lipolytica as a platform for synthesis of drop-in transportation fuels and oleochemicals. Proc. Natl. Acad. Sci. USA. 113, 10848-10853.
88. Yang, K., Qiao, Y., Li, F., Xu, Y., Yan, Y., Madzak, C., Yan, J., 2019. Subcellular engineering of lipase dependent pathways directed towards lipid related organelles for highly effectively compartmentalized biosynthesis of triacylglycerol derived products in Yarrowia lipolytica. Metab. Eng. 55, 231-238.
89. Yuan, J., Ching, C.-B., 2016. Mitochondrial acetyl-CoA utilization pathway for terpenoid productions. Metab. Eng. 38, 303-309.
90. Zhang, C., Seow, V. Y., Chen, X., Too, H.-P., 2018. Multidimensional heuristic process for high-yield production of astaxanthin and fragrance molecules in Escherichia coli. Nat. Commun. 9, 1858.
91. Zhou, P., Li, M., Shen, B., Yao, Z., Bian, Q., Ye, L., Yu, H., 2019. Directed coevolution of β-carotene ketolase and hydroxylase and its application in temperature-regulated biosynthesis of astaxanthin. J. Agric. Food. Chem. 67, 1072-1080.
92. Zhou, P., Xie, W., Li, A., Wang, F., Yao, Z., Bian, Q., Zhu, Y., Yu, H., Ye, L., 2017. Alleviation of metabolic bottleneck by combinatorial engineering enhanced astaxanthin synthesis in Saccharomyces cerevisiae. Enzyme Microb. Technol. 100, 28-36.
93. Zhou, P., Ye, L., Xie, W., Lv, X., Yu, H., 2015. Highly efficient biosynthesis of astaxanthin in Saccharomyces cerevisiae by integration and tuning of algal crtZ and bkt. Appl. Microbiol. Biotechnol. 99, 8419-8428.
94. Zhou, X., Gong, Z., Su, Y., Lin, J., Tang, K., 2009. Cordyceps fungi: natural products, pharmacological functions and developmental products. J. Pharm. Pharmacol. 61, 279-291.
95. Zhou, Y. J., Buijs, N. A., Zhu, Z., Gómez, D. O., Boonsombuti, A., Siewers, V., Nielsen, J., 2016. Harnessing yeast peroxisomes for biosynthesis of fatty-acid-derived biofuels and chemicals with relieved side-pathway competition. J. Am. Chem. Soc. 138, 15368-15377.
96. Chen Y, Nielsen J. Advances in metabolic pathway and strain engineering paving the way for sustainable production of chemical building blocks. Curr. Opin. Biotechnol. 24, 965-972 (2013).
97. Li Y, Smolke C D. Engineering biosynthesis of the anticancer alkaloid noscapine in yeast. Nat. Commun. 7, 12137 (2016).
98. Liu Y, Nielsen J. Recent trends in metabolic engineering of microbial chemical factories. Curr. Opin. Biotechnol. 60, 188-197 (2019).
99. Wu G, Yan Q, Jones J A, Tang Y J, Fong S S, Koffas M A. Metabolic burden: cornerstones in synthetic biology and metabolic engineering applications. Trends Biotechnol. 34, 652-664 (2016).
100. Keasling J D. Manufacturing molecules through metabolic engineering. Science 330, 1355-1358 (2010).
101. Reed M C, Lieb A, Nijhout H F. The biological significance of substrate inhibition: a mechanism with diverse functions. Bioessays 32, 422-429 (2010).
102. Boutin J A, Chatelain-Egger F, Vella F, Delagrange P, Ferry G. Quinone reductase 2 substrate specificity and inhibition pharmacology. Chem. Biol. Interact. 151, 213-228 (2005).
103. Belzer M, Morales M, Jagadish B, Mash E A, Wright S H. Substrate-dependent ligand inhibition of the human organic cation transporter OCT2. J. Pharmacol. Exp. Ther. 346, 300-310 (2013).
104. Singh R K, Tiwari M K, Singh R, Lee J-K. From protein engineering to immobilization: promising strategies for the upgrade of industrial enzymes. Int. J. Mol. Sci. 14, 1232-1277 (2013).
105. Mateo C, et al. Immobilization of lactase from Kluyveromyces lactis greatly reduces the inhibition promoted by glucose. Full hydrolysis of lactose in milk. Biotechnol. Prog. 20, 1259-1262 (2004).
106. Daugulis A J, Tomei M C, Guieysse B. Overcoming substrate inhibition during biological treatment of monoaromatics: recent advances in bioprocess design. Appl. Microbiol. Biotechnol. 90, 1589-1608 (2011).
107. Gao F, Daugulis A J. Bioproduction of the aroma compound 2-phenylethanol in a solid-liquid two-phase partitioning bioreactor system by Kluyveromyces marxianus. Biotechnol. Bioeng. 104, 332-339 (2009).
108. Nielsen D R, Amarasiriwardena G S, Prather K L. Predicting the adsorption of second generation biofuels by polymeric resins with applications for in situ product recovery (ISPR). Bioresour. Technol. 101, 2762-2769 (2010).
109. Kim P Y, Pollard D J, Woodley J M. Substrate supply for effective biocatalysis. Biotechnol. Prog. 23, 74-82 (2007).
110. Chen C, et al. Structure-based mutational studies of substrate inhibition of betaine aldehyde dehydrogenase BetB from Staphylococcus aureus. Appl. Environ. Microbiol. 80, 3992-4002 (2014).
111. Shang Y-P, Chen Q, Li A-T, Quan S, Xu J-H, Yu H-L. Attenuated substrate inhibition of a haloketone reductase via structure-guided loop engineering. J. Biotechnol. 308, 141-147 (2020).
112. Verdoes J C, Misawa N, van Ooyen A J. Cloning and characterization of the astaxanthin biosynthetic gene encoding phytoene desaturase of Xanthophyllomyces dendrorhous. Biotechnol. Bioeng. 63, 750-755 (1999).
113. Verdoes J, Krubasik P, Sandmann G, Van Ooyen A. Isolation and functional characterisation of a novel type of carotenoid biosynthetic gene from Xanthophyllomyces dendrorhous. Mol. Gen. Genet. 262, 453-461 (1999).
114. Velayos A, Blasco J L, Alvarez M I, Iturriaga E A, Eslava A P. Blue-light regulation of phytoene dehydrogenase (carB) gene expression in Mucor circinelloides. Planta 210, 938-946 (2000).
115. Velayos A, Eslava A P, Iturriaga E A. A bifunctional enzyme with lycopene cyclase and phytoene synthase activities is encoded by the carRP gene of Mucor circinelloides. Eur. J. Biochem. 267, 5509-5519 (2000).
116. Yang J, Anishchenko I, Park H, Peng Z, Ovchinnikov S, Baker D. Improved protein structure prediction using predicted interresidue orientations. Proc. Natl. Acad. Sci. USA 117, 1496-1503 (2020).
117. Ro D-K, et al. Production of the antimalarial drug precursor artemisinic acid in engineered yeast. Nature 440, 940-943 (2006).
118. Westfall P J, et al. Production of amorphadiene in yeast, and its conversion to dihydroartemisinic acid, precursor to the antimalarial agent artemisinin. Proc. Natl. Acad. Sci. USA 109, E111-E118 (2012).
119. Chatzivasileiou A O, Ward V, Edgar S M, Stephanopoulos G. Two-step pathway for isoprenoid synthesis. Proc. Natl. Acad. Sci. USA 116, 506-511 (2019).
120. Clomburg J M, Qian S, Tan Z, Cheong S, Gonzalez R. The isoprenoid alcohol pathway, a synthetic route for isoprenoid biosynthesis. Proc. Natl. Acad. Sci. USA 116, 12810-12815 (2019).
121. Rico J, et al. Exploring natural biodiversity to expand access to microbial terpene synthesis. Microb. Cell Fact. 18, 23 (2019).
122. Lund S, Hall R, Williams G J. An artificial pathway for isoprenoid biosynthesis decoupled from native hemiterpene metabolism. ACS Synth. Biol. 8, 232-238 (2019).
123. Qiao K, Wasylenko™, Zhou K, Xu P, Stephanopoulos G. Lipid production in Yarrowia lipolytica is maximized by engineering cytosolic redox metabolism. Nat. Biotechnol. 35, 173 (2017).
124. Ma T, et al. Lipid engineering combined with systematic metabolic engineering of Saccharomyces cerevisiae for high-yield production of lycopene. Metab. Eng. 52, 134-142 (2019).
125. Larroude M, Celinska E, Back A, Thomas S, Nicaud J M, Ledesma-Amaro R. A synthetic biology approach to transform Yarrowia lipolytica into a competitive biotechnological producer of β-carotene. Biotechnol. Bioeng. 115, 464-472 (2018).
126. Braunwald T, et al. Effect of different C/N ratios on carotenoid and lipid production by Rhodotorula glutinis. Appl. Microbiol. Biotechnol. 97, 6581-6588 (2013).
127. Somashekar D, Joseph R. Inverse relationship between carotenoid and lipid formation in Rhodotorula gracilis according to the C/N ratio of the growth medium. World J. Microbiol. Biotechnol. 16, 491-493 (2000).
128. Wang W, et al. Harnessing the intracellular triacylglycerols for titer improvement of polyketides in Streptomyces. Nat. Biotechnol. 38, 76-83 (2020).
129. Xu P, Qiao K, Ahn W S, Stephanopoulos G. Engineering Yarrowia lipolytica as a platform for synthesis of drop-in transportation fuels and oleochemicals. Proc. Natl. Acad. Sci. USA 113, 10848-10853 (2016).
130. Reetz M T. Biocatalysis in organic chemistry and biotechnology: past, present, and future. J. Am. Chem. Soc. 135, 12480-12496 (2013).
131. Tan S Z, Manchester S, Prather K L. Controlling central carbon metabolism for improved pathway yields in Saccharomyces cerevisiae. ACS Synth. Biol. 5, 116-124 (2016).
132. Zhao E M, et al. Optogenetic regulation of engineered cellular metabolism for microbial chemical production. Nature 555, 683 (2018).
133. Brockman I M, Prather K L. Dynamic knockdown of E. coli central metabolism for redirecting fluxes of primary metabolites. Metab. Eng. 28, 104-113 (2015).
134. Schwartz C M, Hussain M S, Blenner M, Wheeldon I. Synthetic RNA polymerase III promoters facilitate high-efficiency CRISPR-Cas9-mediated genome editing in Yarrowia lipolytica. ACS Synth. Biol. 5, 356-359 (2016).
135. Millard P, Letisse F, Sokol S, Portais J-C. IsoCor: correcting M S data in isotope labeling experiments. Bioinformatics 28, 1294-1296 (2012).
136. Asker D. Isolation and characterization of a novel, highly selective astaxanthin-producing marine bacterium. J. Agric. Food. Chem. 65, 9101-9109 (2017).
137. Pompon D, Louerat B, Bronine A, Urban P. Yeast expression of animal and plant P450s in optimized redox environments. Methods Enzymol. 272, 51-64 (1996).
138. Altschul S F, et al. Gapped BLAST and PSI-BLAST: a new generation of protein database search programs. Nucleic Acids Res. 25, 3389-3402 (1997).
139. Suzek B E, Huang H, McGarvey P, Mazumder R, Wu C H. UniRef: comprehensive and non-redundant UniProt reference clusters. Bioinformatics 23, 1282-1288 (2007).
140. Pedregosa F, et al. Scikit-learn: Machine learning in Python. J. Mach. Learn. Res. 12, 2825-2830 (2011).
141. Guindon S, Dufayard J-F, Lefort V, Anisimova M, Hordijk W, Gascuel O. New algorithms and methods to estimate maximum-likelihood phylogenies: assessing the performance of PhyML 3.0. Syst. Biol. 59, 307-321 (2010).
142. Larroude M, Celinska E, Back A, Thomas S, Nicaud J M, Ledesma—Amaro R. A synthetic biology approach to transform Yarrowia lipolytica into a competitive biotechnological producer of β-carotene. Biotechnol. Bioeng. 115, 464-472 (2018).
143. Gao S, et al. Iterative integration of multiple-copy pathway genes in Yarrowia lipolytica for heterologous β-carotene production. Metab. Eng. 41, 192-201 (2017).
144. Xie W, Ye L, Lv X, Xu H, Yu H. Sequential control of biosynthetic pathways for balanced utilization of metabolic intermediates in Saccharomyces cerevisiae. Metab. Eng. 28, 8-18 (2015).
145. Yang J, Guo L. Biosynthesis of β-carotene in engineered E. coli using the MEP and MVA pathways. Microb. Cell Fact. 13, 160 (2014).
146. Zhao J, et al. Engineering central metabolic modules of Escherichia coli for improving β-carotene production. Metab. Eng. 17, 42-50 (2013).
147. Li Y, et al. Metabolic engineering of Escherichia coli using CRISPR-Cas9 meditated genome editing. Metab. Eng. 31, 13-21 (2015).
148. Luo Z, et al. Enhancing isoprenoid synthesis in Yarrowia lipolytica by expressing the isopentenol utilization pathway and modulating intracellular hydrophobicity. Metab. Eng. 61, 344-351 (2020).
149. hi B, et al. Systematic metabolic engineering of Saccharomyces cerevisiae for lycopene overproduction. J. Agric. Food. Chem. 67, 11148-11157 (2019).
150. Ma T, et al. Lipid engineering combined with systematic metabolic engineering of Saccharomyces cerevisiae for high-yield production of lycopene. Metab. Eng. 52, 134-142 (2019).
151. Kang W, et al. Modular enzyme assembly for enhanced cascade biocatalysis and metabolic flux. Nat. Commun. 10, 1-11 (2019).
152. Chen Y, Xiao W, Wang Y, Liu H, Li X, Yuan Y. Lycopene overproduction in Saccharomyces cerevisiae through combining pathway engineering with host engineering. Microb. Cell Fact. 15, 113 (2016).
153. Xie W, Lv X, Ye L, Zhou P, Yu H. Construction of lycopene-overproducing Saccharomyces cerevisiae by combining directed evolution and metabolic engineering. Metab. Eng. 30, 69-78 (2015).
154. Sun T, et al. Production of lycopene by metabolically-engineered Escherichia coli. Biotechnol. Lett. 36, 1515-1522 (2014).

What is claimed is:

1. A genetically modified yeast cell (modified cell) comprising:
(i) a heterologous gene, wherein the heterologous gene encodes an enzyme having beta-galactosidase (LacA) activity;
(ii) one or more heterologous genes encoding one or more enzymes capable of converting lactic acid to pyruvate;
(iii) one or more heterologous genes encoding one or more enzymes of the Leloir pathway; and
(iv) one or more heterologous genes encoding one or more enzymes of the mevalonate pathway.

2. The modified cell of claim 1, wherein the modified cell is an oleaginous yeast cell.

3. The modified cell of claim 2, wherein the oleaginous cell is a Yarrowia lipolytica cell.

4. The modified cell of any one of claims 1-3, wherein the one or more heterologous genes encoding one or more enzymes capable of converting lactic acid to pyruvate is/are selected from the group consisting of a lactate transporter (JEN1) and lactate dehydrogenase (LDH).

5. The modified cell of any one of claims 1-4, wherein the one or more heterologous genes encoding one or more enzymes of the Leloir pathway is/are selected from the group consisting of GAL10M, GAL1, GAL7, and GAL10E.

6. The modified cell of any one of claims 1-5, wherein the one or more heterologous genes encoding one or more enzymes of the mevalonate pathway is/are selected from the group consisting of GGPPS, CarRP, and CarB.

7. The modified cell of claim 6, wherein the GGPPS is GGPPSxd derived from Xanthophyllomyces dendrorhous, GGPPSsa derived from Sulfolobus acidocaldarius, GGPPStc derived from Taxus canadensis, GGPPSpa derived from Pantoea agglomerans, GGPPSyl derived from Yarrowia lipolytica.

8. The modified cell of any one of claims 1-7, further comprising a heterologous gene encoding an enzyme having lycopene beta cyclase activity.

9. The modified cell of claim 8, wherein the enzyme having lycopene beta cyclase activity comprises an amino acid sequence at least 90% identical to the amino acid sequence as set forth in SEQ ID NO: 1.

10. The modified cell of claim 9, wherein the enzyme having lycopene beta cyclase activity comprises an amino acid sequence set forth in any one of SEQ ID NOs: 2-4.:

11. The modified cell of claim 10, further comprising a heterologous gene encoding tHMGR, ERG12, IDI, and ERG20 of the Mevalonate (MVA) pathway, and/or Choline Kinase (CK) and Isopentenyl Phosphate Kinase (IPK).

12. The modified cell of any one of claims 1-8, further comprising:
(i) a heterologous gene encoding an enzyme having beta-carotene ketolase (CrtW) activity; and
(ii) a heterologous gene encoding an enzyme having beta-carotene hydroxylase (CrtZ) activity.

13. The modified cell of claim 12, wherein the enzyme having CrtW activity is fused to the enzyme having CrtZ activity (CrtW/CrtZ fusion enzyme).

14. The modified cell of claim 13, wherein the CrtW/CrtZ fusion enzyme comprises a localization signal.

15. The modified cell of claim 14, wherein the localization signal targets the CrtW/CrtZ fusion enzyme to the endoplasmic reticulum, peroxisome, and/or lipid bodies.

16. The modified cell of any one of claims 1-15, further comprising a heterologous gene encoding an enzyme having lycopene beta cyclase activity and/or a heterologous gene encoding an enzyme having lycopene epsilon cyclase activity.

17. The modified cell of claim 16, wherein the enzyme having lycopene beta cyclase activity comprises an amino acid sequence at least 90% identical to the amino acid sequence as set forth in SEQ ID NO: 1.

18. The modified cell of claim 16 or 17, further comprising a heterologous gene encoding an enzyme having carotenoid hydroxylase 1 (LUT1) activity and/or a heterologous gene encoding an enzyme having carotenoid hydroxylase 5 (LUT5) activity.

19. A genetically modified yeast cell (modified cell) comprising:
(i) a first heterologous gene, wherein the first heterologous gene encodes an enzyme having beta-carotene ketolase (CrtW) activity; and
(ii) a second heterologous gene, wherein the second heterologous gene encodes an enzyme having beta-carotene hydroxylase (CrtZ) activity;
wherein the modified cell produces beta-carotene.

20. The modified cell of claim 19, wherein the modified cell is an oleaginous yeast cell.

21. The modified cell of claim 20, wherein the oleaginous cell is a Yarrowia lipolytica cell.

22. The modified cell of any one of claims 19-21, wherein the enzyme having CrtW activity is fused to the enzyme having CrtZ activity.

23. The modified cell of claim 22, wherein the CrtW/CrtZ fusion enzyme comprises a localization signal.

24. The modified cell of claim 23, wherein the localization signal targets the CrtW/CrtZ fusion enzyme to the endoplasmic reticulum, peroxisome, and/or lipid bodies.

25. A method of converting a carbon source to lycopene and/or beta-carotene, comprising:
(i) contacting a modified cell according to any one of claims 1-11 with a carbon source; and
(ii) incubating the modified cell with the carbon source for a sufficient time to convert the carbon source to lycopene and/or beta-carotene.

26. The method of claim 25, wherein the carbon source is acid whey.

27. The method of claim 25 or 26, wherein the carbon source is converted to lycopene.

28. The method of claim 25 or 26, wherein the carbon source is converted to beta-carotene.

29. A method of converting a carbon source to astaxanthin, comprising:
(i) contacting a modified cell according to any one of claims 12-15 or 19-24 with a carbon source; and
(ii) incubating the modified cell with the carbon source for a sufficient time to convert the carbon source to astaxanthin.

30. The method of claim 29, wherein the carbon source is acid whey.

31. A method of converting a carbon source to alpha-carotene, comprising:
(i) contacting a modified cell according to any one of claims 1-18 with a carbon source; and
(ii) incubating the modified cell with the carbon source for a sufficient time to convert the carbon source to alpha-carotene.

32. The method of claim 31, wherein the carbon source is acid whey.

33. A method of converting a carbon source to lutein, comprising:
(i) contacting a modified cell according to any one of claims 1-18 with a carbon source; and
(ii) incubating the modified cell with the carbon source for a sufficient time to convert the carbon source to lutein.

34. The method of claim 33, wherein the carbon source is acid whey.

35. An enzyme having lycopene beta cyclase activity comprising an amino acid sequence set forth in SEQ ID NO: 2.

36. An enzyme having lycopene beta cyclase activity comprising an amino acid sequence set forth in SEQ ID NO: 3.

37. An enzyme having lycopene beta cyclase activity comprising an amino acid sequence set forth in SEQ ID NO: 4.


SEQUENCE LISTING (85 sequences)

SEQ ID NO: 1 - protein, 237 aa, synthetic construct
MLLTYMEVHLYYTLPVLGVLSWLSRPYYTATDALKFKFLTLVAFTTASAWDNYIVYHKAWSYCPTCVTAVIGYVPLEEYMFFIIMTLLTVAFTNLVMRWHLHSFFIRPETPVMQSVLVRLVPITALLITAYKAWHLAVPGKPLFYGSCILWYACPVLALLWFGAGEYMMRRPLAVLVSIALPTLFLCWVDVVAIGAGTWDISLATSTGKFVVPHLPVEEFMFFALINTVLVFGTCAI

SEQ ID NO: 2 - protein, 237 aa, synthetic construct
MLLTYMEVHLYYTLPVLGVLSWLSRPRYTATDALKFKFLTLVAFTTASAWDNYIVYHKAWSYCPTCVTAVIGYVPLEEYMFFIIMTLLTVAFTNLVMRWHLHSFFIRPETPVMQSVLVRLVPITALLITAYKAWHLAVPGKPLFYGSCILWYACPVLALLWFGAGEYMMRRPLAVLVSIALPTLFLCWVDVVAIGAGTWDISLATSTGKFVVPHLPVEEFMFFALINTVLVFGTCAI

SEQ ID NO: 3 - protein, 237 aa, synthetic construct
MLLTYMEVHLYYTLPVLGVLSWLSRPYYTATDALKFKFLTLVAFTTASAWDNYIVYHKAWSYCPTCVTAVIGYVPLEEYMFFIIMTLLTVAFTNLVMRWHLHSFFIRPETPVMQSVLVRLVPITALLITAYKAWHLAVPGKPLFYGSCILWYACPVLALLWFGAGEYMMRRPLAWLVSIALPTLFLCWVDVVAIGAGTWDISLATSTGKFVVPHLPVEEFMFFALINTVLVFGTCAI

SEQ ID NO: 4 - protein, 237 aa, synthetic construct
MLLTYMEVHLYYTLPVLGVLSWLSRPYYTARDALKFKFLTLVAFTTASAWDNYIVYHKAWSYCPTCVTAVIGYVPLEEYMFFIIMTLLTVAWTNLVMRWHLHSFFIRPETPVMQSVLVRLVPITALLITAYKAWHLAVPGKPLFYGSCILWYACPVLALLWFGAGEYMMRRPLAVLVSIALPTLFLCWVDVVAIGAGTWDISLATSTGKFVVPHLPVEEFMFFALINTVLVFGTCAI

SEQ ID NO: 5 - protein, 10 aa, synthetic construct
GGGGSGGPGS

SEQ ID NO: 6 - DNA, 12 bp, synthetic construct
  source 1..12: mol_type = other DNA
aaggacgagctg

SEQ ID NO: 7 - DNA, 40 bp, synthetic construct
  source 1..40: mol_type = other DNA
atgaaccagatgcatagcacttaagactcgcctcgccaca

SEQ ID NO: 8 - DNA, 40 bp, synthetic construct
  source 1..40: mol_type = other DNA
acacaagacatatctacagcatgaccgccgctgtggctga

SEQ ID NO: 9 - DNA, 40 bp, synthetic construct
  source 1..40: mol_type = other DNA
atgaaccagatgcatagcacttaggccagagcggggacca

SEQ ID NO: 10 - DNA, 40 bp, synthetic construct
  source 1..40: mol_type = other DNA
acacaagacatatctacagcatgcacgtggcctctgctct

SEQ ID NO: 11 - DNA, 40 bp, synthetic construct
  source 1..40: mol_type = other DNA
atgaaccagatgcatagcacttaggcggtgtcgcccttag

SEQ ID NO: 12 - DNA, 40 bp, synthetic construct
  source 1..40: mol_type = other DNA
acacaagacatatctacagcatgtctgcccacgctctgcc

SEQ ID NO: 13 - DNA, 40 bp, synthetic construct
  source 1..40: mol_type = other DNA
ttttgcagtactaaccgcaggcctggctgacctggatcgc

SEQ ID NO: 14 - DNA, 40 bp, synthetic construct
  source 1..40: mol_type = other DNA
gcaagaccggcaacgtggggttaggcgccagaagaagagg

SEQ ID NO: 15 - DNA, 40 bp, synthetic construct
  source 1..40: mol_type = other DNA
ttttgcagtactaaccgcagctgtctaagctgcagtctat

SEQ ID NO: 16 - DNA, 40 bp, synthetic construct
  source 1..40: mol_type = other DNA
gcaagaccggcaacgtggggttatcgcttagaccagtcca

SEQ ID NO: 17 - DNA, 40 bp, synthetic construct
  source 1..40: mol_type = other DNA
ttttgcagtactaaccgcagctgtggatctggaacgccct

SEQ ID NO: 18 - DNA, 40 bp, synthetic construct
  source 1..40: mol_type = other DNA
gcaagaccggcaacgtggggttacttgccagaggcgggct

SEQ ID NO: 19 - DNA, 45 bp, synthetic construct
  source 1..45: mol_type = other DNA
cgggtccgccagagcctccgccgcctcgcttagaccagtccagct

SEQ ID NO: 20 - DNA, 45 bp, synthetic construct
  source 1..45: mol_type = other DNA
cggaggctctggcggacccggctctatgtctgcccacgctctgcc

SEQ ID NO: 21 - DNA, 40 bp, synthetic construct
  source 1..40: mol_type = other DNA
gcaagaccggcaacgtggggttaggcggtgtcgcccttag

SEQ ID NO: 22 - DNA, 40 bp, synthetic construct
  source 1..40: mol_type = other DNA
ttttgcagtactaaccgcagtctgcccacgctctgcccaa

SEQ ID NO: 23 - DNA, 45 bp, synthetic construct
  source 1..45: mol_type = other DNA
cgggtccgccagagcctccgccgccggcggtgtcgcccttagttc

SEQ ID NO: 24 - DNA, 45 bp, synthetic construct
  source 1..45: mol_type = other DNA
cggaggctctggcggacccggctctatgctgtctaagctgcagtc

SEQ ID NO: 25 - DNA, 45 bp, synthetic construct
  source 1..45: mol_type = other DNA
cggaggctctggcggacccggctctatggccgaccgagaccgaag

SEQ ID NO: 26 - DNA, 40 bp, synthetic construct
  source 1..40: mol_type = other DNA
gcaagaccggcaacgtggggttaggaggaagctcgaccac

SEQ ID NO: 27 - DNA, 57 bp, synthetic construct
  source 1..57: mol_type = other DNA
gcaagaccggcaacgtggggttacagctcgtcctttcgcttagaccagtccagctcg

SEQ ID NO: 28 - DNA, 54 bp, synthetic construct
  source 1..54: mol_type = other DNA
gcaagaccggcaacgtggggttacagcttggatcgcttagaccagtccagctcg

SEQ ID NO: 29 - DNA, 561 bp, synthetic construct
  source 1..561: mol_type = other DNA
atggccgaccgagaccgaagcggcatctacggcggcgcccacgctacctacggccagcagcagcagcagggcggcggtggtcgacctatgggcgagcaggtcaagggcatgctgcacgacaagggccccactgcctcccaggccctcaccgttgccaccctgttccccctgggcggcctccttctcgtgctttccggtcttgccctgactgcctccgtggtcggcctggctgtcgccactcccgtcttcctcatcttctcccccgtcctcgtccccgccgcccttcttatcggcaccgccgtcatgggcttcctgacctctggcgccctgggcctcggcggtctttcctctctcacctgccttgccaacaccgcccgacaggcctttcagcgaacccccgactacgtcgaggaggcccaccgacgaatggccgaggccgctgctcacgccggtcacaagaccgcccaggccggacaggccatccagggacgagcccaggaggccggtgccggtggtggtgctggtgctggagccggaggcggtggtcgagcttcctcctaa

SEQ ID NO: 30 - RNA, 20 nt, Yarrowia lipolytica
  source 1..20: mol_type = genomic RNA
  modified_base 12: mod_base = OTHER; note = Thymine
  modified_base 15: mod_base = OTHER; note = Thymine
acgccgaggagtggtaccgg

SEQ ID NO: 31 - DNA, 60 bp, synthetic construct
  source 1..60: mol_type = other DNA
gggtcggcgcaggttgacgtacgccgaggagtggtaccgggttttagagctagaaatagc

SEQ ID NO: 32 - DNA, 60 bp, synthetic construct
  source 1..60: mol_type = other DNA
gctatttctagctctaaaacccggtaccactcctcggcgtacgtcaacctgcgccgaccc

SEQ ID NO: 33 - DNA, 40 bp, synthetic construct
  source 1..40: mol_type = other DNA
atgaaccagatgcatagcacttagaaggccaggatgccca

SEQ ID NO: 34 - DNA, 40 bp, synthetic construct
  source 1..40: mol_type = other DNA
catcaacagtatctacacgcatgggcaaggaacaggacca

SEQ ID NO: 35 - DNA, 40 bp, synthetic construct
  source 1..40: mol_type = other DNA
cattcaaaggatcccctaggatgaccgctctggcctacta

SEQ ID NO: 36 - DNA, 40 bp, synthetic construct
  source 1..40: mol_type = other DNA
gcaagaccggcaacgtggggttactggccctcccagccag

SEQ ID NO: 37 - DNA, 40 bp, synthetic construct
  source 1..40: mol_type = other DNA
atgaaccagatgcatagcacttagatcacgttagagttgt

SEQ ID NO: 38 - DNA, 40 bp, synthetic construct
  source 1..40: mol_type = other DNA
catcaacagtatctacacgcatgtccaagaagcacatcgt

SEQ ID NO: 39 - DNA, 40 bp, synthetic construct
  source 1..40: mol_type = other DNA
cattcaaaggatcccctaggatgctgctgacctacatgga

SEQ ID NO: 40 - DNA, 40 bp, synthetic construct
  source 1..40: mol_type = other DNA
gcaagaccggcaacgtggggttagatggtgttcaggtttc

SEQ ID NO: 41 - DNA, 40 bp, synthetic construct
  source 1..40: mol_type = other DNA
cattcaaaggatcccctaggatggactacgccaacatcct

SEQ ID NO: 42 - DNA, 40 bp, synthetic construct
  source 1..40: mol_type = other DNA
gcaagaccggcaacgtggggttacaggggaatgtcggcca

SEQ ID NO: 43 - DNA, 42 bp, synthetic construct
  source 1..42: mol_type = other DNA
gcaagaccggcaacgtggggttacagctcttgtcgcacgtcg

SEQ ID NO: 44 - DNA, 30 bp, synthetic construct
  source 1..30: mol_type = other DNA
tcgctcgaggccccacttgtagccgtccag

SEQ ID NO: 45 - DNA, 30 bp, synthetic construct
  source 1..30: mol_type = other DNA
acaagtggggcctcgagcgacgatctatcc

SEQ ID NO: 46 - DNA, 40 bp, synthetic construct
  source 1..40: mol_type = other DNA
cattcaaaggatcccctaggatgcagccccactacgacct

SEQ ID NO: 47 - DNA, 40 bp, synthetic construct
  source 1..40: mol_type = other DNA
gcaagaccggcaacgtggggttatcggtgagtggtcatga

SEQ ID NO: 48 - DNA, 40 bp, synthetic construct
  source 1..40: mol_type = other DNA
cattcaaaggatcccctaggatgccccgatacgacctgat

SEQ ID NO: 49 - DNA, 40 bp, synthetic construct
  source 1..40: mol_type = other DNA
gcaagaccggcaacgtggggttactgcatggcctgctgtc

SEQ ID NO: 50 - DNA, 40 bp, synthetic construct
  source 1..40: mol_type = other DNA
cattcaaaggatcccctaggatgtcttacgacctgatcct

SEQ ID NO: 51 - DNA, 40 bp, synthetic construct
  source 1..40: mol_type = other DNA
gcaagaccggcaacgtggggttaagatcgcatctcgaagt

SEQ ID NO: 52 - DNA, 40 bp, synthetic construct
  source 1..40: mol_type = other DNA
cattcaaaggatcccctaggatgctgtctcccctgcagcg

SEQ ID NO: 53 - DNA, 40 bp, synthetic construct
  source 1..40: mol_type = other DNA
gcaagaccggcaacgtggggttacttgatcatggctggag

SEQ ID NO: 54 - DNA, 40 bp, synthetic construct
  source 1..40: mol_type = other DNA
cattcaaaggatcccctaggatggcctacaccgccatggc

SEQ ID NO: 55 - DNA, 40 bp, synthetic construct
  source 1..40: mol_type = other DNA
gcaagaccggcaacgtggggttagttctgtcggaaggcaa

SEQ ID NO: 56 - DNA, 40 bp, synthetic construct
  source 1..40: mol_type = other DNA
cattcaaaggatcccctaggatggtctctggatctaaggc

SEQ ID NO: 57 - DNA, 40 bp, synthetic construct
  source 1..40: mol_type = other DNA
gcaagaccggcaacgtggggtcaggcaatcttcatgacag

SEQ ID NO: 58 - DNA, 40 bp, synthetic construct
  source 1..40: mol_type = other DNA
cattcaaaggatcccctaggatggattataacagcgcgga

SEQ ID NO: 59 - DNA, 40 bp, synthetic construct
  source 1..40: mol_type = other DNA
gcaagaccggcaacgtggggtcactgcgcatcctcaaagt

SEQ ID NO: 60 - DNA, 40 bp, synthetic construct
  source 1..40: mol_type = other DNA
cattcaaaggatcccctaggatgtcctacttcgacaacta

SEQ ID NO: 61 - DNA, 40 bp, synthetic construct
  source 1..40: mol_type = other DNA
gcaagaccggcaacgtggggttactttcgtcgtcgaatgg

SEQ ID NO: 62 - DNA, 40 bp, synthetic construct
  source 1..40: mol_type = other DNA
atgaaccagatgcatagcacctaatgggtccagggaccga

SEQ ID NO: 63 - DNA, 40 bp, synthetic construct
  source 1..40: mol_type = other DNA
acacaagacatatctacagcatggactacatcatttcggc

SEQ ID NO: 64 - DNA, 40 bp, synthetic construct
  source 1..40: mol_type = other DNA
ttttgcagtactaaccgcagacccagtctgtgaaggtggt

SEQ ID NO: 65 - DNA, 40 bp, synthetic construct
  source 1..40: mol_type = other DNA
gcaagaccggcaacgtggggctatgaccgtatgcaaatat

SEQ ID NO: 66 - DNA, 40 bp, synthetic construct
  source 1..40: mol_type = other DNA
atgaaccagatgcatagcacctacttctgtcgcttgtaaa

SEQ ID NO: 67 - DNA, 40 bp, synthetic construct
  source 1..40: mol_type = other DNA
acacaagacatatctacagcatgtccaaggcgaaattcga

SEQ ID NO: 68 - DNA, 40 bp, synthetic construct
  source 1..40: mol_type = other DNA
ttttgcagtactaaccgcagacgacgtcttacagcgacaa

SEQ ID NO: 69 - DNA, 40 bp, synthetic construct
  source 1..40: mol_type = other DNA
gcaagaccggcaacgtggggctacttgatccaccgccgaa

SEQ ID NO: 70 - DNA, 40 bp, synthetic construct
  source 1..40: mol_type = other DNA
atgaaccagatgcatagcacctacgcattaataaaccggg

SEQ ID NO: 71 - DNA, 40 bp, synthetic construct
  source 1..40: mol_type = other DNA
acacaagacatatctacagcatggcccctcaacccgacca

SEQ ID NO: 72 - DNA, 40 bp, synthetic construct
  source 1..40: mol_type = other DNA
ttttgcagtactaaccgcaggaactgaacatctctgagtc

SEQ ID NO: 73 - DNA, 40 bp, synthetic construct
  source 1..40: mol_type = other DNA
gcaagaccggcaacgtggggttacttggagaatcggatga

SEQ ID NO: 74 - DNA, 18 bp, synthetic construct
  source 1..18: mol_type = other DNA
gagtcaccggtatcgttc

SEQ ID NO: 75 - DNA, 18 bp, synthetic construct
  source 1..18: mol_type = other DNA
gcggagttggtgaaagag

SEQ ID NO: 76 - DNA, 20 bp, synthetic construct
  source 1..20: mol_type = other DNA
ctggccctgctgtggttcgg

SEQ ID NO: 77 - DNA, 20 bp, synthetic construct
  source 1..20: mol_type = other DNA
caggcagatggggcaccacg

SEQ ID NO: 78 - RNA, 24 nt, Yarrowia lipolytica
  source 1..24: mol_type = genomic RNA
  modified_base 13: mod_base = OTHER; note = Thymine
  modified_base 16: mod_base = OTHER; note = Thymine
  modified_base 22: mod_base = OTHER; note = Thymine
gacgccgaggagtggtaccggtgg

SEQ ID NO: 79 - protein, 7 aa, Yarrowia lipolytica
DAEEWYR

SEQ ID NO: 80 - RNA, 23 nt, synthetic construct
  source 1..23: mol_type = other RNA
  modified_base 13: mod_base = OTHER; note = Thymine
  modified_base 16: mod_base = OTHER; note = Thymine
  modified_base 21: mod_base = OTHER; note = Thymine
gacgccgaggagtggtccggtgg

SEQ ID NO: 81 - protein, 7 aa, synthetic construct
DAEEWSG

SEQ ID NO: 82 - protein, 7 aa, unidentified
  source 1..7: note = Mucor circinelloides
PLEEYMF

SEQ ID NO: 83 - DNA, 21 bp, unidentified
  source 1..21: mol_type = genomic DNA; note = Mucor circinelloides
cccctggaagagtacatgttc

SEQ ID NO: 84 - protein, 7 aa, synthetic construct
PLEKYMF

SEQ ID NO: 85 - DNA, 21 bp, synthetic construct
  source 1..21: mol_type = other DNA
cccctggaaaagtacatgttc